(19) United States
(12) Patent Application Publication
(10) Pub. No.: US 2026/0226462 A1
(43) Pub. Date: Aug. 6, 2026

(54) SYNTHETIC MRNA LACKING A POLYA TAIL OR HAVING A SHORT ADENINE HOMOPOLYMER AND METHODS OF USE AND PRODUCTION THEREOF

(71) Applicant: Elixirgen Therapeutics, Inc., Baltimore, MD (US)

(72) Inventor: Minoru S.H. KO, Cockeysville, MD (US)

(73) Assignee: Elixirgen Therapeutics, Inc., Baltimore, MD (US)

(21) Appl. No.: 19/153,924

(22) PCT Filed: Feb. 14, 2024
(86) PCT No.: PCT/US2024/015869

§ 371 Date: Aug. 5, 2025

Related U.S. Application Data
(60) Provisional application No. 63/484,969, filed on Feb. 14, 2023.
(60) Provisional application No. 63/502,030, filed on May 12, 2023.
(60) Provisional application No. 63/518,310, filed on Aug. 8, 2023.
(60) Provisional application No. 63/586,985, filed on Sep. 29, 2023.

(51) Int. Cl. C12N 15/113; C07K 14/47; C07K 14/78

(52) U.S. Cl. CPC C12N 15/113; C07K 14/4707; C07K 14/78; C12N 2310/317; C12N 2310/532; C12N 2770/24122; C12N 2830/50

(57) ABSTRACT

The present disclosure relates to mRNA molecules that include a 3′-untranslated region (3′-UTR) of a positive-sense, single-stranded RNA (+ssRNA) of a virus, a coding sequence for a protein that is heterologous to the virus, and a 5′-untranslated region (5′-UTR). In particular, the present disclosure relates to mRNA encoding a protein of interest but lacking a poly(A) tail. Furthermore, the present disclosure relates to such mRNAs that permit the addition of an adenine homopolymer at their 3′ ends.

### CROSS-REFERENCE TO RELATED APPLICATION(S)

This application claims priority benefit of U.S. Provisional Patent Application Nos. 63/586,985 filed Sep. 29, 2023, 63/518,310 filed Aug. 8, 2023, 63/502,030 filed May 12, 2023, and 63/484,969 filed Feb. 14, 2023, all of which are hereby incorporated by reference in their entirety.

### REFERENCE TO AN ELECTRONIC SEQUENCE LISTING

The content of the electronic sequence listing (699442001840SEQLIST.xml; Size: 99,514 bytes; and Date of Creation: Feb. 13, 2024) is herein incorporated by reference in its entirety.

### FIELD

The present disclosure relates to mRNA molecules that include a 3′-untranslated region (3′-UTR) of a positive-sense, single-stranded RNA (+ssRNA) of a virus, a coding sequence for a protein that is heterologous to the virus, and a 5′-untranslated region (5′-UTR). In particular, the present disclosure relates to mRNA encoding a protein of interest but lacking a poly(A) tail. Furthermore, the present disclosure relates to such mRNAs that permit the addition of an adenine homopolymer at their 3′ ends.

### BACKGROUND

Synthetic messenger RNA (synRNA) is now frequently used in therapeutic products, as well as in general tools for biomedical research. Basic structural components of mRNA include a 5′-Cap, a 5′-UTR (5′-untranslated region), a CDS (coding sequence), a 3′-UTR (3′-untranslated region), and a poly(A) tail (see, e.g., Fang et al., 2022; Kowalski et al., 2019; Jackson et al., 2020; and Wadhwa et al., 2019). Poly(A) tails are homopolymers of adenine (A) of about 100-300 nucleotides in length. It is known that mRNAs with longer poly(A) tails are more stable and produce more proteins (see, e.g., Nicholson and Pasquinelli, 2019; and Fang et al., 2022). In addition, synRNA lacking a poly(A) tail has been shown to be unsuitable for protein production (Holtkamp et al., 2006).

As a properly manufactured therapeutic product, it is important to ensure the identity and consistency of a synRNA between manufacturing batches. To this end, one of the technical hurdles is to ensure that each synRNA molecule has identical sequences. Especially problematic part is the poly(A) tail since synRNA is frequently present in mixtures of mRNA molecules with different poly(A) lengths. This is particularly a problem for synRNAs encoding large proteins such as dystrophin, which has a coding region of over 11 kb.

As such, there is a need in the art for tools for production of translatable mRNA lacking a poly(A) tail.

Furthermore, some methods of purifying synRNAs use an oligo(dT) column to bind to poly(A) tails of synRNAs (Mencin et al., 2023). For this purpose, short poly(A) tails of 10 to 20 adenines are sufficient (Mencin et al., 2023). However, it is not known whether +ssRNA virus sequences permit the addition of short poly(A) tails, as in their natural form some of these sequences lack poly(A) tails.

As such, there is also a need in the art for tools for production of translatable mRNA having a short adenine homopolymer at its 3′ end.

### BRIEF SUMMARY

The present disclosure relates to mRNA molecules that include a 3′-untranslated region (3′-UTR) of a positive-sense, single-stranded RNA (+ssRNA) of a virus, a coding sequence for a protein that is heterologous to the virus, and a 5′-untranslated region (5′-UTR). In particular, the present disclosure relates to mRNA encoding a protein of interest but lacking a poly(A) tail. Furthermore, the present disclosure relates to such mRNAs that permit the addition of an adenine homopolymer at their 3′ ends.

### BRIEF DESCRIPTION OF THE DRAWINGS

FIG. 1A-1E depict the structures of RNA molecules and a DNA template for production of RNA molecules. The synthetic mRNAs (synRNAs) are based on the 5′ and 3′ untranslated regions (UTRs) of Nodamura virus RNA genome 1 (NOV1, NCBI Accession: NC_002690) and RNA genome 2 (NOV2, NCBI Accession: NC_002691), respectively. FIG. 1A shows a schematic representation of NOV1-EGFP synRNA (SEQ ID NO:1), in which the CDS (coding sequence) of NOV1 RNA is replaced with the coding sequence of enhanced green fluorescent protein (EGFP). FIG. 1B shows a schematic representation of NOV2-EGFP synRNA (SEQ ID NO:2), in which the CDS of NOV2 RNA is replaced with the coding sequence of EGFP. FIG. 1C shows a schematic representation of NOV2m synRNA (SEQ ID NO:3), in which the CDS of NOV2 RNA is replaced with a multiple cloning site (MCS). FIG. 1D shows a schematic representation of NOV2m-EGFP synRNA (SEQ ID NO:4), in which the coding sequence of EGFP is inserted in the MCS site of NOV2m RNA. FIG. 1E shows a schematic representation of a plasmid DNA, which can be used as a template for the production of synRNA by in vitro transcription (IVT). The coding sequence of EGFP is set forth as SEQ ID NO:5, and the coding sequence of an exemplary MCS is set forth as SEQ ID NO:8.

FIG. 2A-2B depict a comparison of EGFP expression between NOV1-EGFP synRNA and NOV2-EGFP synRNA in human neonate dermal fibroblast cells (HDFn). NOV1-EGFP synRNA and NOV2-EGFP synRNA were prepared by in vitro transcription driven by T7 RNA polymerase from plasmid DNA linearized with a SapI restriction enzyme. The 5′-Cap was incorporated using CleanCap AG (TriLink). Two version of synRNAs were prepared: one RNA was a standard RNA without any nucleoside modifications (Unmodified), and the other RNA was modified with 5-methylcytosine (5mC) and pseudouridine (Ψ). About 0.5 μg of RNAs were transfected into HDFn with MessengerMax transfection reagent (ThermoFisher). HDFn cells were cultured in a standard culture condition at 37° C. (FIG. 2A) and a reduced temperature condition at 33° C. (FIG. 2B) with or without B18R. Phase-contrast (left) and fluorescent (right) images were taken at 12 hours after RNA transfection. Fluorescent images showed EGFP expression levels.

FIG. 3 depicts a comparison of the cytopathic effect (CPE) between NOV1-EGFP synRNA and NOV2-EGFP synRNA in human neonate dermal fibroblast cells (HDFn). NOV1-EGFP synRNA and NOV2-EGFP synRNA were prepared by in vitro transcription driven by T7 RNA polymerase from plasmid DNA linearized with a SapI restriction enzyme. The 5′-Cap was incorporated using CleanCap AG (TriLink). Two version of RNAs were prepared: one RNA was a standard RNA without any nucleoside modifications (Unmodified), and the other RNA was modified by 5-methylcytosine (5mC) and pseudouridine (Ψ). About 0.5 μg of RNAs were transfected into HDFn with MessengerMax transfection reagent (ThermoFisher). HDFn cells were cultured in a standard culture condition at 37° C. and 33° C. with or without B18R. Phase-contrast images were taken at 96 hours after RNA transfection.

FIG. 4A-4B compares the percentages of HDFn cells transfected with NOV2-EGFP synRNA (m1Ψ), NOV2m-EGFP synRNA (m1Ψ), or Control-EGFP synRNA (5mC+Ψ). About 1 μg of RNAs were transfected into HDFn with MessengerMax transfection reagent (ThermoFisher). HDFn cells were cultured in a standard culture condition at 37° C. in the presence of B18R (B18R+; upper panel) and in the absence of B18R (B18R−; lower panel), and EGFP expression levels (fluorescence intensities) were measured by Moxi GO II (ORFLO) at 24 hours, 96 hours, and 185 hours after transfection. In FIG. 4A, GFP-positive (+) cells were presented as a percentage (%) of total cells at the fluorescence intensity of GFP>30 or GFP>300 (10-fold above threshold). FIG. 4B provides representative phase-contrast and fluorescent images of NOV2-EGFP transfected cells and shows flow cytometry dot plots with EGFP gating boxes. These data were used to produce FIG. 4A.

FIG. 5A-5C depict the structures of RNA molecules of DENVm and Capless-DENVm, and the predicted secondary structure of DENVm RNA with the locations of mutated nucleotides indicated by arrows. The synthetic mRNAs (synRNAs) are based on the 5′ and 3′ untranslated regions (UTRs) of Dengue virus 2 (DENV) (NC_001474.2; Kinney et al., 1997). FIG. 5A shows a schematic representation of DENVm synRNA (SEQ ID NO:11), in which the CDS (coding sequence) of DENV RNA is replaced with a multiple cloning site (MCS or m).

FIG. 5B shows a schematic representation of Capless-DENVm synRNA (SEQ ID NO:12), in which the 5′Cap of DENVm synRNA is removed. FIG. 5C shows a schematic representation of predicted secondary structure of DENVm synRNA. The prediction was made using the RNAfold WebServer tool available from the web site of the University of Vienna, Institute for Theoretical Chemistry (Reuter and Mathews, 2010). Four mutations were introduced to remove two ATG start codons of DENV, upstream of MCS, and to preserve the secondary structure.

FIG. 6 shows the percentages of GFP-positive HDFn cells transfected with DENVm-EGFP synRNA (U: unmodified) and DENVm-EGFP synRNA (m1Ψ). 1 μg of RNAs each were transfected into HDFn with MessengerMax transfection reagent (ThermoFisher). HDFn cells were cultured in a standard culture condition at 30° C. or 37° C. in the presence or absence of B18R. EGFP expression levels (fluorescence intensities) were measured by Moxi GO II (ORFLO) on Day 1 and Day 4 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 7 shows the percentages of GFP-positive HDFn cells transfected with Capless-DENVm-EGFP synRNA (U: unmodified) and Capless-DENVm-EGFP synRNA (m1Ψ). 1 μg of RNAs each were transfected into HDFn with MessengerMax transfection reagent (ThermoFisher). HDFn cells were cultured in a standard culture condition at 30° C. or 37° C. in the presence or absence of B18R. EGFP expression levels (fluorescence intensities) were measured by Moxi GO II (ORFLO) on Day 1 and Day 4 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 8 shows the percentages of GFP-positive HDFn cells transfected with DENVm-EGFP synRNA (U: unmodified) and DENVm-EGFP synRNA (m1Ψ). 1 μg of RNAs each were transfected into HDFn with MessengerMax transfection reagent (ThermoFisher). HDFn cells were cultured in a standard culture condition at 33° C. or 37° C. in the presence or absence of B18R. EGFP expression levels (fluorescence intensities) were measured by Moxi GO II (ORFLO) on Day 1, Day 4, Day 8, and Day 11 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 9 depicts the schematic presentation of synRNA molecules of Capless-BYDVm, Capless-BYDV2m, Capless-MNESVm, Capless-TCVm, Capless-PMVm, and Capless-PEMV2m. These synRNAs do not have either 5′-Cap or 3′-poly(A) tail. Capless-DENVm (also in FIG. 5) is shown here again for comparison. The size (kb) of each synRNA molecule is also shown. Capless-BYDVm RNA (SEQ ID NO:14) consists of 5′-UTR and 3′-UTR of Barley yellow dwarf virus (BYDV: NC_004750.1) with additional MCS. Capless-BYDV2m RNA (SEQ ID NO:16) consists of 5′-UTR, BYDV-like translation element (BTE), and 3′-UTR of Barley yellow dwarf virus (BYDV: NC_004750.1) with additional MCS. Capless-MNESVm RNA (SEQ ID NO:18) consists of 5′-UTR and 3′-UTR of Maize necrotic streak virus (MNESV: NC_007729.1) with additional MCS. Capless-TCVm RNA (SEQ ID NO:24) consists of 5′-UTR and 3′-UTR of Turnip crinkle virus RNA for coat protein (TCV: NC_003821.3) with additional MCS. Capless-PMVm RNA (SEQ ID NO:20) consists of 5′-UTR and 3′-UTR of Panicum mosaic virus (PMV: U55002.1) with additional MCS. Capless-PEMV2m RNA (SEQ ID NO:22) consists of 5′-UTR and 3′-UTR of Pea enation mosaic virus-2 (PEMV2: NC_003853.1) with additional MCS.

FIG. 10 depicts the schematic presentation of synRNA molecules of BYDVm, BYDV2m, MNESVm, TCVm, PMVm, and PEMV2m. These synRNAs have 5′-Cap, but do not have 3′-poly(A) tail. The size (kb) of each synRNA molecule is also shown. BYDVm RNA (SEQ ID NO:13) consists of 5′-UTR and 3′-UTR of Barley yellow dwarf virus (BYDV: NC_004750.1) with additional 5′-Cap and MCS. BYDV2m RNA (SEQ ID NO:15) consists of 5′-UTR, BYDV-like translation element (BTE), and 3′-UTR of Barley yellow dwarf virus (BYDV: NC_004750.1) with additional 5′-Cap and MCS. MNESVm RNA (SEQ ID NO:17) consists of 5′-UTR and 3′-UTR of Maize necrotic streak virus (MNESV: NC_007729.1) with additional 5′-Cap and MCS. TCVm RNA (SEQ ID NO:23) consists of 5′-UTR and 3′-UTR of Turnip crinkle virus RNA for coat protein (TCV: NC_003821.3) with additional 5′-Cap and MCS. PMVm RNA (SEQ ID NO:19) consists of 5′-UTR and 3′-UTR of Panicum mosaic virus (PMV: U55002.1) with additional 5′-Cap and MCS. PEMV2m RNA (SEQ ID NO:21) consists of 5′-UTR and 3′-UTR of Pea enation mosaic virus-2 (PEMV2: NC_003853.1) with additional 5′-Cap and MCS.

FIG. 11 shows the percentages of GFP-positive HDFn cells transfected with synRNAs with 5′-Cap, but poly(A)less, and cultured at 33° C. in the presence of B18R. The synRNAs used: poly(A)less Control-EGFP synRNA (m1Ψ), DENVm-EGFP (m1Ψ), DENVm-EGFP (Unm, Unmodified), NOV2m-EGFP (m1Ψ), NOV2-EGFP (m1Ψ), PEMV2m-EGFP (m1Ψ), PEMV2m-EGFP (Unm), PMVm-EGFP (m1Ψ), PMVm-EGFP (Unm), MNESVm-EGFP (m1Ψ), MNESVm-EGFP (Unm), BYDV2m-EGFP (m1Ψ), BYDV2m-EGFP (Unm), TCVm-EGFP (m1Ψ), TCVm-EGFP (Unm), BYDm-EGFP (m1Ψ), BYDm-EGFP (Unm). EGFP expression levels (fluorescence intensities) were measured by Moxi GO II (ORFLO) on Day 1, and Day 4 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 12 shows the percentages of GFP-positive HDFn cells transfected with synRNAs with 5′-Cap, but poly(A)less, and cultured at 33° C. in the absence of B18R. The synRNAs used: poly(A)less Control-EGFP synRNA (m1Ψ), DENVm-EGFP (m1Ψ), DENVm-EGFP (Unm, Unmodified), NOV2m-EGFP (m1Ψ), NOV2-EGFP (m1Ψ), PEMV2m-EGFP (m1Ψ), PEMV2m-EGFP (Unm), PMVm-EGFP (m1Ψ), PMVm-EGFP (Unm), MNESVm-EGFP (m1Ψ), MNESVm-EGFP (Unm), BYDV2m-EGFP (m1Ψ), BYDV2m-EGFP (Unm), TCVm-EGFP (m1Ψ), TCVm-EGFP (Unm), BYDm-EGFP (m1Ψ), BYDm-EGFP (Unm). EGFP expression levels (fluorescence intensities) were measured by Moxi GO II (ORFLO) on Day 1, and Day 4 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 13 shows the percentages of GFP-positive HDFn cells transfected with synRNAs with 5′-Cap, but poly(A)less, and cultured at 37° C. in the presence of B18R. The synRNAs used: poly(A)less Control-EGFP synRNA (m1Ψ), DENVm-EGFP (m1Ψ), DENVm-EGFP (Unm, Unmodified), NOV2m-EGFP (m1Ψ), NOV2-EGFP (m1Ψ), PEMV2m-EGFP (m1Ψ), PEMV2m-EGFP (Unm), PMVm-EGFP (m1Ψ), PMVm-EGFP (Unm), MNESVm-EGFP (m1Ψ), MNESVm-EGFP (Unm), BYDV2m-EGFP (m1Ψ), BYDV2m-EGFP (Unm), TCVm-EGFP (m1Ψ), TCVm-EGFP (Unm), BYDm-EGFP (m1Ψ), BYDm-EGFP (Unm). EGFP expression levels (fluorescence intensities) were measured by Moxi GO II (ORFLO) on Day 1, and Day 4 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 14 shows the percentages of GFP-positive HDFn cells transfected with synRNAs with 5′-Cap, but poly(A)less, and cultured at 37° C. in the absence of B18R. The synRNAs used: poly(A)less Control-EGFP synRNA (m1Ψ), DENVm-EGFP (m1Ψ), DENVm-EGFP (Unm, Unmodified), NOV2m-EGFP (m1Ψ), NOV2-EGFP (m1Ψ), PEMV2m-EGFP (m1Ψ), PEMV2m-EGFP (Unm), PMVm-EGFP (m1Ψ), PMVm-EGFP (Unm), MNESVm-EGFP (m1Ψ), MNESVm-EGFP (Unm), BYDV2m-EGFP (m1Ψ), BYDV2m-EGFP (Unm), TCVm-EGFP (m1Ψ), TCVm-EGFP (Unm), BYDm-EGFP (m1Ψ), BYDm-EGFP (Unm). EGFP expression levels (fluorescence intensities) were measured by Moxi GO II (ORFLO) on Day 1, and Day 4 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 15 presents the summarized view of TCVm-EGFP (Unm), TCVm-EGFP (m1Ψ), MNESVm-EGFP (Unm), MNESVm-EGFP (m1Ψ), and poly(A)less Control-EGFP synRNA (m1Ψ) data, presented in FIGS. 11, 12, 13, and 14. GFP-positive (+) cells were presented as a percentage (%) of total live cells at the fluorescence intensity of GFP>30, GFP>300 (10-fold above threshold), or GFP>2000 (66.7-fold above threshold).

FIG. 16 presents representative microscopic images of experiments depicted in FIGS. 11-15. About 1.0 μg of RNAs were transfected into HDFn cells with MessengerMax transfection reagent (ThermoFisher). HDFn cells were cultured in a reduced temperature condition at 33° C. (upper panel) and standard culture condition at 37° C. (lower panel) and with or without B18R. Phase-contrast (left) and fluorescent (right) images were taken at 24 hours after RNA transfection. Fluorescent images showed EGFP expression levels.

FIG. 17 presents representative results of fluorescence activated cell sorter (FACS) analyses, comparing EGFP fluorescence intensities among the poly(A)less TCVm-EGFP mRNA (top), Control-EGFP synRNA with the standard 120 poly(A) tail described in Warren et al., 2010 (middle), and non-transfected control (bottom). About 1.0 μg of RNAs were transfected into HDFn cells with MessengerMax transfection reagent (ThermoFisher). HDFn cells were cultured in a standard culture condition at 37° C. with B18R. Cells were harvested 16 hours after mRNA transfection and subjected to the FACS analysis. Geometric mean fluorescence intensity (MFI) showed that the translation efficiency of TCVm mRNA without poly(A) tail was comparable to that of the standard mRNA.

FIG. 18A presents representative bioluminescent images of luciferase assays in mice one day or two days after intramuscular injection of 20 μg synRNA-encoding luciferase (LUC2) with InvivoFectamine3.0 (ThermoFisher) In FIG. 18B, luciferase activities were assessed by quantifying bioluminescence signals. TCVm and NOV2m RNAs were used as synRNA-encoding LUC2. Because SapI restriction enzyme site was present in LUC2, plasmid DNAs were linearized by MluI, which was present at the immediate 3′-side of SapI site. As a negative control, NOV2m-LUC synRNA generated after SapI linearization of the plasmid DNA was used as a background signal (shown as a dotted line in FIG. 18B). Two mice strains, C57BL/6 and BALB/c were used. TCVm-LUC2 (MluI) was highly translated in vivo, even without a poly(A) tail. NOV2m-LUC2 (MluI) also worked, though the translation efficiency was lower than that of TCVm-LUC2. Thus, poly(A)less synRNA was translatable in both C57BL/6 and BALB/c strains of mice.

FIG. 19 presents representative microscopic images of experiments to test the effect of addition of poly(A) tails to the 3′-end of poly(A)-tailless mRNAs. Poly(A)less Control-EGFP synRNA (m1Ψ) and its added versions (20As, 30As, 60As, and 120As). A120 version was identical to synRNA described in Warren et al., 2010, except the modification with m1Ψ. NOV2m-EGFP (m1Ψ) and its adenine homopolymer-added versions (20As, 30As, 60As, and 120As). MNESVm-EGFP (Nucleoside-unmodified) and its adenine homopolymer-added versions (20As, 30As, 60As, and 120As). TCVm-EGFP (m1Ψ) and its adenine homopolymer-added versions (20As, 30As, 60As, and 120As). About 1.0 μg of RNAs were transfected into HDFn cells with MessengerMax transfection reagent (ThermoFisher). HDFn cells were cultured in a reduced temperature condition at 33° C. with (upper panel) or without B18R (lower panel). Fluorescent images were taken at 24 hours after RNA transfection. Fluorescent images showed EGFP expression levels.

FIG. 20 presents representative microscopic images of experiments as described in FIG. 19 except that HDFn cells were cultured in a standard temperature condition at 37° C. with (upper panel) or without B18R (lower panel). Fluorescent images were taken at 24 hours after RNA transfection. Fluorescent images showed EGFP expression levels.

FIG. 21 shows the percentages of GFP-positive HDFn cells in the experiments described in FIG. 19. HDFn cells were cultured in a reduced temperature condition at 33° C. with (left panel) or without B18R (right panel). EGFP expression levels (fluorescence intensities) were measured by Moxi GO II (ORFLO) on Day 1 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 22 shows the percentages of GFP-positive HDFn cells in the experiments described in FIG. 20. HDFn cells were cultured in a standard temperature condition at 37° C. with (left panel) or without B18R (right panel). EGFP expression levels (fluorescence intensities) were measured by Moxi GO II (ORFLO) on Day 1 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 23A shows a schematic representation of NOV2m-DMD-A28 synRNA, which encodes a full-length human dystrophin (DMD) protein (the coding region of transcript variant Dp427m is shown as nucleotides 238-11295 of NCBI accession number NM_004006). The mRNA sequence of NOV2m-DMD-A28 synRNA is set forth as SEQ ID NO:42. FIG. 23B shows the results of immunohistochemistry using an antibody against human DMD (MANDYS106, Millipore). Nuclei were visualized with 4′,6-diamidino-2-phenylindole (DAPI). HDFn cells were transfected with NOV2m-DMD-A28 synRNA using MessengerMax transfection reagent (ThermoFisher). HDFn cells were cultured at 37° C. with 250 ng/mL of B18R (Sigma), for 24 hours after synRNA transfection and subjected to immunohistochemistry.

FIG. 24A shows a schematic representation of NOV2m-LUC-DMD-A28 synRNA, which encodes for a fusion protein of luciferase (LUC) and a full-length human dystrophin (DMD) protein (transcript variant Dp427m, NCBI accession number NM_004006).

FIG. 24B shows representative bioluminescent images of luciferase assays in mice. Rep. 1 and Rep. 2 indicate replicates. FIG. 24C shows the luciferase activities assessed by the Bioluminescent Imaging system. In brief, 20.0 μg of NOV2m-LUC-DMD-A28 synRNA was complexed with Lipid Nanoparticle (LNP: InvivoFectamine3.0, ThermoFisher) according to the manufacturer's protocol. The synRNA/LNP complexes were directly injected into muscles in the right thigh region of BALB/c mice (Day 0). The following day (Day 1), luciferase activity was monitored by the AMI HTX Bioluminescent Imaging system (Spectral Instruments Imaging, Tucson, AZ).

FIG. 25A shows a schematic representation of NOV2m-COL7A1-LUC-A28 synRNA, which encodes a fusion protein of a full-length human collagen type VII alpha-1 (VII) chain (COL7A1) protein (the coding region of COL7A1 is shown as nucleotides 64-8898 of NCBI accession number NM_000094) and a luciferase (LUC) gene. FIG. 25B shows the results of immunohistochemistry using an antibody against human COL7A1 (MCA597GA, BioRad). Nuclei were visualized with DAPI. HDFn cells were transfected once (lx transfection) or three times (3× transfection) with NOV2m-COL7A1-LUC-A28 synRNA using MessengerMax transfection reagent (ThermoFisher). HDFn cells were cultured at 37° C. with 250 ng/mL of B18R (Sigma), for 24 hours after synRNA transfection (lx transfection) or for 72 hours after the first synRNA transfection (3× transfection). Samples were then subjected to immunohistochemistry.

FIG. 26 shows the percentages of GFP-positive HDFn cells transfected with synRNAs in which a poly(A) tail was added to the 3′-end of poly(A)-tailless mRNAs. The synRNAs used: DENVm-EGFP (Unm: Unmodified nucleosides) and its adenine homopolymer-added versions (20As, 30As, 60As, and 120As). About 1.0 μg of RNAs were transfected into HDFn cells with MessengerMax transfection reagent (ThermoFisher). HDFn cells were cultured in a reduced temperature condition at 33° C. or in a standard culture condition at 37° C., with or without B18R. EGFP expression levels (fluorescence intensities) were measured by Moxi GO II (ORFLO) on Day 2 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 27A shows a schematic representation of synRNA molecules of SARSVm. The synRNA has 5′-Cap, 5′-UTR and 3′-UTR of severe acute respiratory syndrome coronavirus-2 (SARS-CoV-2: NC_045512.2), multiple cloning sites (MCS), and 50 adenine homopolymer. The nucleotide sequence of the SARSVm 5′-UTR is set forth as SEQ ID NO:37, and the nucleotide sequence of the SARSVm 3′-UTR+50(A) is set forth as SEQ ID NO:38. FIG. 27B shows the percentages of GFP-positive HDFn cells transfected with SARSVm synRNA. Both nucleoside-unmodified natural synRNA (Unm) and nucleoside-modified synRNA (m1Ψ) were tested. About 1.0 μg of RNAs were transfected into HDFn cells with MessengerMax transfection reagent (ThermoFisher). HDFn cells were cultured in a reduced temperature condition at 33° C. or in a standard culture condition at 37° C., with or without B18R. EGFP expression levels (fluorescence intensities) were measured by Moxi GO II (ORFLO) on Day 1 after transfection. GFP-positive (+) cells were presented as a percentage (%) of total live cells at the fluorescence intensity of GFP>30, GFP>300 (10-fold above threshold), or GFP>2000 (66.7-fold above threshold).

FIG. 28 shows the changes in luciferase activity in BALB/c mice from Day 1 to Day 8 after receiving intramuscular injection of 20 μg each of NOV2m (A50)-LUC synRNA, NOV2m (A30)-LUC synRNA, DENVm (A50)-LUC synRNA, or DENVm (A30)-LUC synRNA. Both nucleoside-unmodified natural synRNA (Unm) and nucleoside-modified synRNA (m1Ψ) were tested. synRNAs were complexed with Invivofectamine3.0 (ThermoFisher) before intramuscular injection. Luciferase activity was assessed by the AMI HTX Bioluminescent Imaging system (Spectral Instruments Imaging, Tucson, AZ). Mean±SEM (n=2) are plotted.

FIG. 29 shows the changes in luciferase activity in BALB/c mice from Day 1 to Day 13 after receiving intradermal injection of 20 μg of modified or unmodified NOV2m (A50)-LUC synRNA, NOV2m (A30)-LUC synRNA, DENVm (A50)-LUC synRNA, or DENVm (A30)-LUC synRNA. synRNAs were dissolved in lactated ringer's solution and delivered to skin without a transfection reagent or lipid nanoparticles (LNPs). To test the effects of chitosan oligosaccharide on gene expression, synRNAs were intradermally injected with [Chitosan (+)] or without [Chitosan (−)] chitosan oligosaccharides (1.5 μg/ml final concentration). Luciferase activity was assessed by the AMI HTX Bioluminescent Imaging system (Spectral Instruments Imaging, Tucson, AZ). Mean±SEM [n=2 for Chitosan (−) condition; n=3 for Chitosan (+) conditions] are plotted.

FIG. 30A shows a schematic representation of a synRNA encoding a fusion protein for Type I collagen, including coding regions for two COL1A1 proteins (1,464 amino acids in length each) and one COL1A2 protein (1,366 amino acids in length), fused via nucleic acids encoding 2A self-cleaving peptides (e.g., T2A and E2A). The nucleotide sequences encoding an exemplary COLlA fusion protein is shown as SEQ ID NO:39. FIG. 30B shows a schematic representation of a synRNA encoding a fusion protein for erythropoietin (EPO) including coding regions for multiple copies of EPO protein (193 amino acids in length), fused via nucleic acids encoding 2A self-cleaving peptides (e.g., F2A, T2A, E2A, and P2A). The nucleotide sequence encoding an exemplary EPO fusion protein is shown as SEQ ID NO:40.

FIG. 30C shows a schematic representation of a synRNA encoding a ribonucleoprotein-telomerase, which is composed of 5′-Cap, 5′-UTR, a telomerase reverse transcriptase (TERT: 1132 amino acids) coding region for the protein component, 3′-UTR, a ribozyme, a telomerase RNA (TERC: 451 nt.) as the RNA component, a ribozyme, and a poly(A) tail. The nucleotide sequence of an exemplary TERT-TERC mRNA is shown in SEQ ID NO:41.

FIG. 31 shows proper localization of full-length human dystrophin protein in mouse skeletal muscle 1 day after injection with 20 μg NOV2m-DMD-A28 synRNA+Invivofectamine (BALB/c mice). Ab (MANDYS106) does not recognize mouse dystrophin, but recognizes human dystrophin, whereas Ab (AB15277) recognizes both mouse and human DMD.

FIG. 32A-32B shows recovery of muscle strength in D2.mdx mutant mice after intramuscular injection of mRNA-DMD. In brief, 20 μg of NOV2m-DMD-A28 synRNA or mRNA-LUC (luciferase: control) was mixed with Invivofactamine (ThermoFisher) in a total volume of 60 μL. D2.mdx mutant mice (lacking mouse dystrophin protein) received 3 intramuscular injections in ventral forearm and 2 intramuscular injections in dorsal forearm of approximately 4 μg (12 μL) of NOV2m-DMD-A28 synRNA or mRNA-LUC using 31G needles for a total 20 μg (in 60 μL) in each of the right forearm and the left forearm. The injection started at 11-week-old and continued once per week for a total of 6 injections. The final injection was at 16-week-old. Peak muscle strength of the forearm was measured by a grip strength meter (Harvard apparatus). The measurement was performed twice 30 minutes apart. Peak muscle strength was normalized by mouse weight and the average of two measurements were used for the analyses. FIG. 32A shows peak muscle strength one week after the final injection (measured at week 17). NOV2m-DMD-A28 injected group showed statistically significant (p<0.05) recovery of muscle strength compared to non-injected group (D2.mdx: control) and mRNA-LUC (luciferase: control) injected group (D2.mdx-LUC). There was no statistically significant difference between the D2.mdx-DMD group and the wildtype DBA/2 group (control). FIG. 32B shows a recovery of muscle strength in D2.mdx mutant mice (lacking dystrophin protein) by a single intramuscular injection of NOV2m-DMD-A28. In brief, 20 μg of NOV2m-DMD-A28 synRNA was mixed with Invivofactamine (ThermoFisher) in a total volume of 60 μL. D2.mdx mutant mice received a single intramuscular injection in ventral forearm at 3 sites and in dorsal forearm at 2 sites of approximately 4 μg (12 μL) of NOV2m-DMD-A28 using 34G needles: a total 20 μg (in 60 μL) in each of the right forearm and the left forearm. The injection was performed at 18-week-old. Three weeks later when mice were 21-weeks-old, peak muscle strength of the forearm was measured by a grip strength meter (Harvard apparatus). The measurement was performed twice 30 minutes apart. Peak muscle strength was normalized by mouse weight and the average of two measurements were used for the analyses. At 21 weeks, the NOV2m-DMD-A28 synRNA injected group (n=5) showed appreciable recovery of muscle strength, whereas the non-injected control group (n=4) did not.

### DETAILED DESCRIPTION

Typically, synthetic mRNAs (synRNAs) have the same sequence features as cellular mRNAs. That is, synRNAs generally include 5′-Cap, 5′-UTR, CDS, 3′-UTR, and poly(A) tail. The present disclosure relates to methods for producing translatable synRNA lacking a poly(A) tail. As described herein, synRNAs based on a positive-sense single-stranded RNA (+ssRNA) virus that naturally lacks a poly(A) tail were designed to remove sequences encoding the viral RNA-dependent RNA polymerase, and thereby, separating the translation function from the replication function of the +ssRNA virus. Expression cassettes based on these designs were inserted into plasmids, which can be used as a template for production of synRNA that is suitable for use in vitro and in vivo. Such synRNAs can be produced by any methods known in the art, including in vitro transcription of template DNA, chemical synthesis of RNA, and plasmid or viral expression vectors. Moreover, synRNAs can be produced with or without modified nucleosides. Additional genetic elements can be incorporated into the synRNAs. In one embodiment, the synRNA comprises a single CDS. In another embodiment, the synRNA comprises multiple CDSs that are constructed by fusing two or more CDSs. In another embodiment, the synRNA comprise multiple CDSs that are linked by an internal ribosome entry site (IRES). In a further embodiment, the synRNA comprises multiple CDSs that are separated by nucleotides encoding a flexible linker (e.g., glycine-serine-linker). In another embodiment, the synRNA comprises multiple CDSs that are separated by nucleotides encoding a 2A self-cleaving peptide (e.g., P2A, E2A, F2A or T2A).

### General Technigues and Defiqitions

The practice of the present disclosure will employ, unless otherwise indicated, conventional techniques of molecular biology (including recombinant techniques), microbiology, cell biology, biochemistry, and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., about 500 nucleotides in length when used in reference to an RNA molecule refers to an RNA molecule that is from 450 to 550 nucleotides in length).

As used herein, the term “synthetic mRNA”, abbreviated as “synRNA” refers to a mRNA molecule comprising at least a 5′-UTR (5′-untranslated region), a CDS (coding sequence), and a 3′-UTR (3′-untranslated region), in which the CDS is heterologous to at least the 3′-UTR. As such, synRNAs are not naturally-occurring molecules.

The term “poly(A) tail”, as used herein, refers to a stretch of at least about 15 consecutive adenine nucleotides, which are typically present at the end of the 3′-UTR of a mRNA molecule. DNA encoding a mRNA molecule native to mammalian cells does not include a homopolymer of adenines at its 3′ end. Rather, adenines are added to the 3′-UTR by polyadenylate polymerase to form a homopolymer of adenines (i.e., a poly(A) tail). The genomes of certain single-stranded, positive-sense RNA (+ssRNA) viruses also lack a homopolymer of adenines at their 3′ ends. The length of the poly(A) tail of a native mRNA molecule depends upon the species of cell in which it was produced, with mammalian cells generally producing mRNAs from genomic DNA with longer poly(A) tails (e.g., usually longer than 100 consecutive adenine nucleotides, such as from about 75 to about 275 consecutive adenine nucleotides in length).

The term “poly(A)-tailless,” “poly(A)less,” and the like, when used in reference to a mRNA molecule refers to a mRNA molecule that does not comprise a poly(A) tail as defined above. In some embodiments, a mRNA devoid of a poly(A) tail has no more than about 10 consecutive adenine residues downstream of its 3′-UTR.

The terms “polypeptide” and “protein” are used interchangeably to refer to a polymer of amino acid residues, and are not limited to a certain length unless otherwise specified. Polypeptides may include natural amino acid residues or a combination of natural and non-natural amino acid residues. The terms also include post-translational modifications of the polypeptide, for example, glycosylation, sialylation, acetylation, phosphorylation, and the like. In some aspects, the polypeptides may contain modifications with respect to a native or natural sequence, as long as the protein maintains the desired activity (e.g., antigenicity).

As used herein in reference to a protein of interest, the terms “coding sequence,” “CDS”, “open reading frame” and “ORF” refer to the nucleotide sequences that encode the protein of interest. Due to the degeneracy of the genetic code, multiple distinct nucleotide sequences can encode the same amino acid sequence.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein. In some embodiments, an isolated protein is at least 75%, 90%, 95%, 96%, 97%, 98% or 99% pure as determined by HPLC.

An “effective amount” or a “sufficient amount” of a substance is that amount sufficient to affect beneficial or desired results, including clinical results, and, as such, an “effective amount” depends upon the context in which it is being applied.

In the present disclosure, the terms “individual” and “subject” refer to mammals. “Mammals” include, but are not limited to, humans, non-human primates (e.g., monkeys), farm animals, sport animals, rodents (e.g., mice and rats) and pets (e.g., dogs and cats). In some preferred embodiments, the subject is a human subject.

The term “dose” as used herein in reference to a composition comprising a mRNA encoding a protein of interest refers to a measured portion of the mRNA taken by (administered to or received by) a subject at any one time.

The relative terms “higher” and “lower” refer to a measurable increase or decrease, respectively, in a response or parameter when compared to otherwise same conditions except for a parameter of interest, or alternatively, as compared to another condition. For instance, the phrases “higher level of protein expression” and “stronger protein expression” refer to a level of protein expression as a consequence of contacting a cell with a composition of the present disclosure comprising a mRNA encoding the protein that is greater than 1, preferably greater than 2, 3, 4, 5, 6, 7, 8, 9, or 10-fold above a level of protein expression as a consequence of a control condition (e.g., administration of a comparator composition that either does not comprise the mRNA or comprises a control mRNA that does not encode the protein). The phrases “lower level of protein expression” and “weaker protein expression” refer to a level of protein expression as a consequence of a control condition (e.g., administration of a comparator composition that either does not comprise the mRNA or comprises a control mRNA that does not encode the protein) that is less than 1, preferably less than 2, 3, 4, 5, 6, 7, 8, 9, or 10-fold below a level of protein expression as a consequence of administration of a composition of the present disclosure comprising an mRNA encoding the protein.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antigen) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into an antigen of interest and the products screened for a desired activity, e.g., increased stability and/or immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

Conservative amino acid substitutions will involve exchanging a member of one of these classes with another member of the same class. Non-conservative amino acid substitutions will involve exchanging a member of one of these classes with a member of another class.

As used herein, the term “excipient” refers to a compound present in a composition comprising an active ingredient (e.g., mRNA encoding a protein of interest). Pharmaceutically acceptable excipients are inert pharmaceutical compounds, and may include for instance, solvents, bulking agents, buffering agents, tonicity adjusting agents, and preservatives (Pramanick et al., Pharma Times, 45:65-77, 2013). In some embodiments the compositions of the present disclosure comprise an excipient that functions as one or more of a solvent, a bulking agent, a buffering agent, and a tonicity adjusting agent (e.g., sodium chloride in saline may serve as both an aqueous vehicle and a tonicity adjusting agent).

### I. SYNTHETIC MRNA (SYNRNA)

In order to establish synRNAs as properly manufactured biologics in CMC (chemistry, manufacturing and control), it is important to ensure the identity of the synRNA and consistency between batches produced. One of the technical hurdles involves ensuring that synRNA molecules in a batch have identical sequences. An especially problematic part of synRNA is the poly(A) tail, a homopolymer that typically includes over 100 adenine (A) nucleotides. Essentially all eukaryotic mRNAs contain a poly(A) tract at their 3′-ends. Mixture of synRNAs with different poly(A) tail lengths are not ideal as therapeutic products.

Three methods are generally used to add a poly(A) tail to the 3′-end of synRNA. One method for adding a poly(A) tail involves the use of a poly(A) polymerase. However, the length of the poly(A) tail cannot be controlled with this method (Holtkamp et al., 2006).

Another method is to add a poly(A) tract by tail-PCR using a primer containing a long stretch of thymine (T) nucleotides at its 5′-end. PCR products are then used as a template for in vitro transcription (IVT) of synRNA by T7-, T3-, or SP6-RNA polymerase (Warren et al., 2010). This method allows the addition of a 120 poly(A) stretch at the 3′-end of synRNA (Warren et al., 2010). Drawbacks of this method include: (1) it is difficult to guarantee that the PCR products have identical sequences; (2) it is difficult to scale up the production of PCR products; and (3) it is difficult to PCR-amplify a long coding sequence.

A further method is to include a poly(A) stretch in a plasmid DNA, which can then be used as a template DNA for in vitro transcription (IVT) of synRNA by T7-, T3-, or SP6-RNA polymerase after linearizing plasmid DNA by restriction enzyme digestion. This method is suitable for scaling up and for maintaining the sequence identity during the amplification in E. coli. Thus, this method overcomes the shortcomings of the tail-PCR method. However, one of the major limitations of this method is that the homopolymer in a plasmid DNA is usually unstable and often truncated during the amplification of plasmid DNA in E. coli. This limits the size of a poly(A) tail to a length shorter than desirable for a poly(A) tail. In an attempt to overcome this problem, a segmented poly(A) tail can be utilized. A segmented poly(A) tail is made up of relatively short poly(A) stretches (e.g., about 50 A nucleotides) that are connected through non-poly(A) linkers (Trepotec et al., 2019). However, this technique does not guarantee the integrity of synRNA. In particular, coding sequences of large proteins of interest increase the size of plasmid DNAs, and inclusion of a lengthy adenine homopolymer results in large plasmids that are even less stable than smaller plasmids.

These issues are resolved by the present disclosure through the design of synRNA molecules that lack poly(A) tails at their 3′-ends. Considering the critical importance of the poly(A) tail for mRNA stability and protein production, it is counter-intuitive to eliminate poly(A) tails from synRNAs. In fact, there are many scientific publications that demonstrate the lack of protein production from mRNAs lacking poly(A) tails (see, e.g., Holtkamp et al., 2006).

During development of the present disclosure, natural examples of functional mRNAs that lack poly(A) tails were considered. Examples of poly(A)-tailless mRNAs are chromosomes of some positive-sense, single-stranded RNA (+ssRNA) viruses. The replication of a +ssRNA virus requires a virus-specific RNA-dependent RNA polymerase (RdRp). However, the RNA genome is usually delivered to cells without the RdRp protein. Therefore, an RNA genome must first be translated to produce the RdRp using host cell translational machinery. Accordingly, many +ssRNA virus chromosomes resemble host cell mRNA in that they contain a 5′-Cap, a 5′-untranslated region (5′-UTR), a coding sequence (CDS), a 3′-UTR, and a poly(A) tail. The CDS of a +ssRNA virus genome must include an open reading frame for the RdRp. Once translated, RdRp replicates the +ssRNA genome, and thus, 5′-UTR, 3′-UTR, and other RNA genome sequences play critical roles, not only in translation, but also in replication. By removing the CDS of RdRp from +ssRNA virus genome, and thereby, separating the translation function from the replication function of +ssRNA, any +ssRNA virus can in principle be converted into a non-replicating synRNA platform.

Interestingly, some +ssRNA viruses do not have poly(A) tail. Examples of +ssRNA virus families that lack a poly(A) tail include, but are not limited to, Nodaviridae (Sahul Hameed et al., 2019), Flaviridae (Simmonds et al., 2017), and Tetraviridae (Dorrington et al., 2009). The Flaviridae family includes, but is not limited to, Flavivirus (for example, yellow fever virus, dengue virus, Zika virus, Japanese encephalitis virus, West Nile virus and tick-borne encephalitis virus), Pestivirus (for example, bovine viral diarrhoea virus and classical swine fever virus) and Hepacivirus (for example, hepatitis C virus), and Pegivirus.

Furthermore, some +ssRNA viruses, primarily for plants, do not have even 5′-Cap structure. Therefore, these +ssRNA viruses are 5′-Cap-less and poly(A)-tailless (Nicholson and White 2011). These +ssRNA viruses employe the mechanism termed 3′-cap-independent translation enhancer (3′-CITE), which are grouped into 6 major classes. Examples of +ssRNA viruses that lack both 5′-Cap and 3′-poly(A) tail include, but are not limited to, Barley yellow dwarf virus (BYDV), Maize necrotic streak virus (MNESV), Panicum mosaic virus (PMV), Pea enation mosaic virus-2 (PEMV2), and Turnip crinkle virus (TCV).

As such the present disclosure provides RNA molecules comprising a 5′-untranslated region (5′-UTR), at least one coding sequence (CDS) for at least one protein, and a 3′-untranslated region (3′-UTR), wherein the 3′-UTR is a virus 3′-UTR or fragment thereof, optionally wherein the fragment is at least 40 nucleotides in length, and the CDS is heterologous to the virus, and replaces an open reading frame of the virus, and the virus is a positive-sense, single-stranded RNA (+ssRNA) virus whose genome (or a chromosome thereof) lacks a poly(A) tail. In some embodiments, the +ssRNA virus is a member of a viral family selected from the group consisting of Nodaviridae, Flaviridae, and Tetraviridae. In some embodiments, the +ssRNA viruses are selected from plant viruses that lack both 5′-Cap and 3′-poly(A) tails, such as Barley yellow dwarf virus (BYDV), Maize necrotic streak virus (MNESV), Panicum mosaic virus (PMV), Pea enation mosaic virus-2 (PEMV2), and Turnip crinkle virus (TCV). However, it was found that the translation efficiency of 5′-Cap-less +ssRNAs was rather low in human cells. Therefore, in some embodiments, Cap structure was artificially added to 5′end of these synRNAs. In some embodiments, an adenine homopolymer of a defined length is added to the 3′ end (synthetic poly(A) tail) of these synRNAs. Ideally, the synthetic poly(A) tail is of a defined length and is shared by a plurality of synRNAs prepared by in vitro transcription. The uniformity in length and sequence of the 3′ adenine homopolymer is beneficial for synRNA purification and for characterization of pharmaceutical compositions comprising the synRNAs.

### II. NODAVIRIDAE CHROMOSOMES

The Nodaviridae family includes, but is not limited to, Nodamuravirus (RNA1[AF174533=NC_002690], RNA2[AF174534=NC_002691]), and Flock House virus (RNA1[X77156=NC_004146], RNA2[X15959=NC_004144]). The genetic component of Nodamura virus (NOV) (Newman 1975) was utilized as a platform for production of poly(A)-tailless synRNA as described in Examples 1-5. However, RNA fragments of other +ssRNA virus, which lack poly(A) tails can also be utilized for production of poly(A)-tailless synRNA.

Nodamuravirus (NOV) is a bipartite RNA virus with two RNA chromosomes: RNA1 encodes RNA-dependent RNA polymerase (RdRp); RNA2 encodes a capsid protein (Hameed 2019). Both NOV1 and NOV2 have 5′-Cap, 5′-UTR, CDS, 3′-UTR, but do not have poly(A) tails (Hameed 2019). Previously, it has been shown that a stem-loop structure at the 3′-UTR is essential for the replication of NOV (Rosskopf, 2010). However, whether the 5′-Cap, 5′-UTR, and 3′-UTR alone are sufficient to drive the efficient translation of CDS, particularly a foreign CDS, has not been investigated.

First, whether NOV1 and NOV2 can be used as a platform for poly(A)less synRNA was tested. The CDS of NOV1 and NOV2 was replaced with EGFP-coding sequence to yield NOV1-EGFP RNA (SEQ ID NO:1) and NOV2-EGFP RNA (SEQ ID NO:2). The RNAs were made by IVT using T7 RNA polymerase and plasmid DNA templates linearized with a SapI restriction enzyme (Example 1). Exemplary plasmid DNA templates contained a T7 promoter; however, other promoters could be used to drive transcription (e.g., T3, SP6, etc.). Exemplary synRNAs included a 5′-Cap, which was added using CleanCap-AG (TriLink). Even so, other methods could be used to add a 5′-Cap in IVT RNA. Although naturally-occurring NOV RNAs do not have any nucleoside modification, both natural version (unmodified) and versions modified with 5mC and Ψ were produced.

NOV1-EGFP RNA and NOV2-EGFP RNA were transfected into human fibroblasts, and viability and expression of EGFP were monitored by microscopy. Both NOV1 and NOV2 untranslated regions were found to be suitable for production of translatable synRNA lacking poly(A) tails (Example 2). Interestingly, NOV2 synRNA resulted in stronger EGFP protein expression than NOV1. This was a surprise since the NOV1 chromosome encodes RdRp, which is used to replicate both NOV1 and NOV2 RNA chromosomes, and hence NOV1 may be expected to work better than NOV2 in terms of initial protein expression. However, the actual results were opposite. Although the 3′-terminal stem-loop structure of 3′-UTR of NOV2 was known to be essential for NOV2 RNA replication (Rosskopf et al., 2010), the role of the NOV2 3′-UTR for translation had not been reported.

Unmodified NOV1-RNA and NOV2-RNA were found to be cytotoxic (Example 2). Many dead cells were observed after transfecting unmodified NOV1-RNA and unmodified NOV2-RNA into human fibroblasts. Addition of a recombinant B18R protein, which is known to suppress innate immunity by counteracting type I Interferon (IFN), was tested as a means for reducing cytotoxicity. Although B18R was found to alleviate the cytotoxicity caused by transfection of cells with NOV1-RNA and NOV2-RNA to some extent and under certain conditions, B18R did not fully alleviate cytotoxicity. Modification of nucleosides by replacing cytosine with 5mC and uridine with Ψ (5mC+Ψ) reduced the cytotoxicity of NOV1 and NOV2 synRNAs (Example 2). Naturally-occurring NOV1 RNA and NOV2 RNA do not contain nucleoside modifications. So, the fact that artificial modifications of nucleosides substantively reduced cytotoxicity of NOV1 and NOV2 RNA was a surprise.

In order to test whether other nucleoside modifications are compatible with NOV2-RNA translation, NOV2-EGFP synRNAs modified with 5mC+Ψ, 5moU, m1Ψ, or Ψ were transfected into human fibroblasts. Viability and EGFP protein expression were monitored by microscopy (Example 3). As observed in the Example 2, unmodified NOV2 synRNAs were cytotoxic, and cytotoxicity was alleviated at least in part by certain nucleoside modifications. Among the tested modifications, 5mC+Ψ, and m1Ψ resulted in the strongest levels of EGFP expression, although the other modifications were also effective to some extent in alleviating cytotoxicity (Example 3).

NOV1 synRNA and NOV2 synRNA were found to be translatable at both 37° C. and 33° C., although the expression was slightly stronger at 37° C. than at 33° C. (Example 2, Example 3). Previous NOV studies have been done at 28° C., 31° C., and 34° C. (Ball et al., 1992; Johnson 2003). As the optimal temperature for NOV replication is around 28° C. and viral replication is greatly reduced at 37° C. (Johnson 2003). As such, higher levels of NOV1 synRNA and NOV2 synRNA were expected when transfected cells were cultured at 33° C., rather than at 37° C. So, it is a surprise to see that NOV1 and NOV2 synRNA were translatable as well or better when transfected cells were cultured at 37° C., than at 33° C.

To further develop NOV2-RNA as a poly(A)less synRNA platform, the effect of additional nucleotides surrounding the ATG start codon and STOP codon on translation of a CDS were tested. For this purpose and to facilitate cloning of plasmid DNA templates for IVT of additional synRNAs, a multiple cloning site was inserted immediately upstream of the ATG start codon and extending through the STOP codon (Example 4). In this way, the ATG-STOP portion of the native NOV2 synRNA sequence was removed. This construct is called NOV2m. An exemplary nucleic acid fragment inserted within the multiple cloning site of NOV2m included a Kozak consensus sequence, an ATG start codon, an EGFP coding sequence, and a Stop codon. The EGFP from NOV2m-EGFP RNA was translated equally well as EGFP from NOV2-EGFP (Example 4, Example 5). Thus, NOV2m is an important tool that simplifies the cloning of any CDS into the multiple cloning site of plasmid DNA, which serves as a template for production of synRNA without a poly(A) tail.

Subsequently, EGFP protein expression from poly(A)less NOV2m-EGFP synRNA was compared to a Control-EGFP synRNA comprising a poly(A) tail of 120A (Example 5). The expression level of EGFP from NOV2m synRNA, which lacks a poly(A) tail, was found to be comparable to the expression level of EGFP from a Control-EGFP synRNA, which includes a 120 nucleotide poly(A) tail.

Accordingly, the present disclosure provides RNA molecules comprising a 5′-untranslated region (5′-UTR), at least one coding sequence (CDS) for at least one protein, and a 3′-untranslated region (3′-UTR), wherein the 3′-UTR is a Nodamura virus RNA2 (NOV2) 3′-UTR or fragment thereof, or a Nodamura virus RNA1 3′-UTR or fragment thereof, optionally wherein the fragment is at least 40 nucleotides in length, and the CDS is heterologous to NOV2 or NOV1, and replaces an open reading frame of a Nodamura virus capsid protein of NOV2 or an open reading frame of a Nodamura virus RNA-dependent RNA polymerase (RdRp).

### III. FLAVIRIDAE CHROMOSOMES

The Flaviridae family includes, but is not limited to, Flavivirus (for example, yellow fever virus, dengue virus, Zika virus, Japanese encephalitis virus, West Nile virus and tick-borne encephalitis virus), Pestivirus (for example, bovine viral diarrhoea virus and classical swine fever virus) and Hepacivirus (for example, hepatitis C virus), and Pegivirus.

As a typical example, the genetic component of Dengue virus 2 (DENV) (NC_001474.2; Kinney et al., 1997) was utilized as a platform for production of poly(A)-tailless synRNA as described in Example 6. However, RNA fragments of other +ssRNA virus, which lack poly(A) tails can also be utilized for production of poly(A)-tailless synRNA.

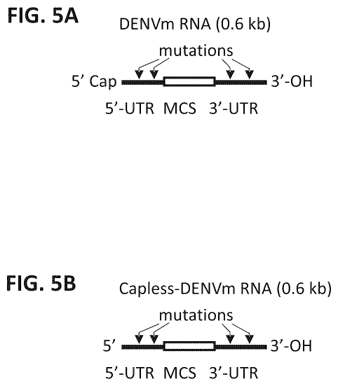

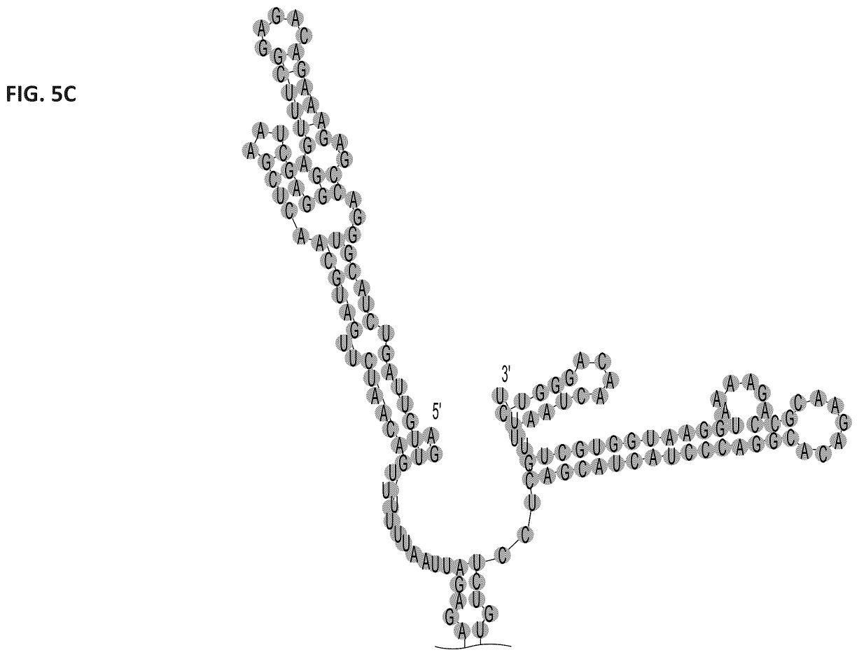

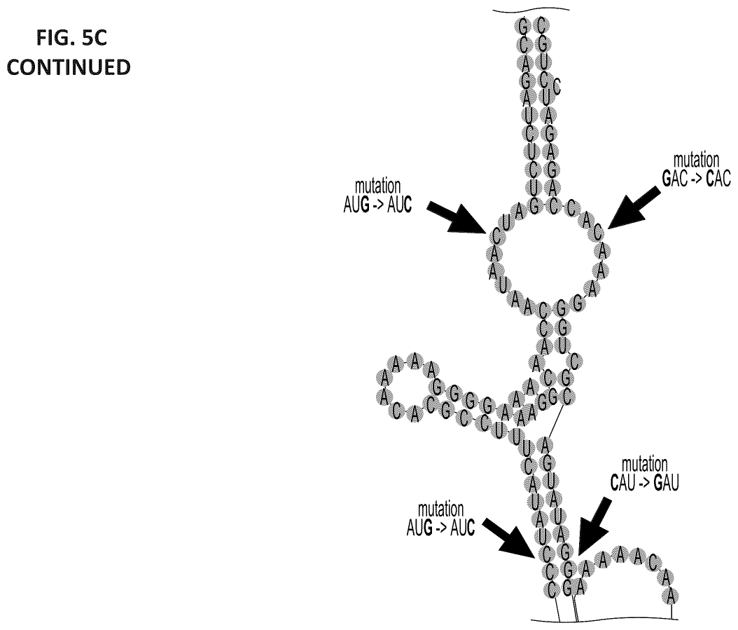

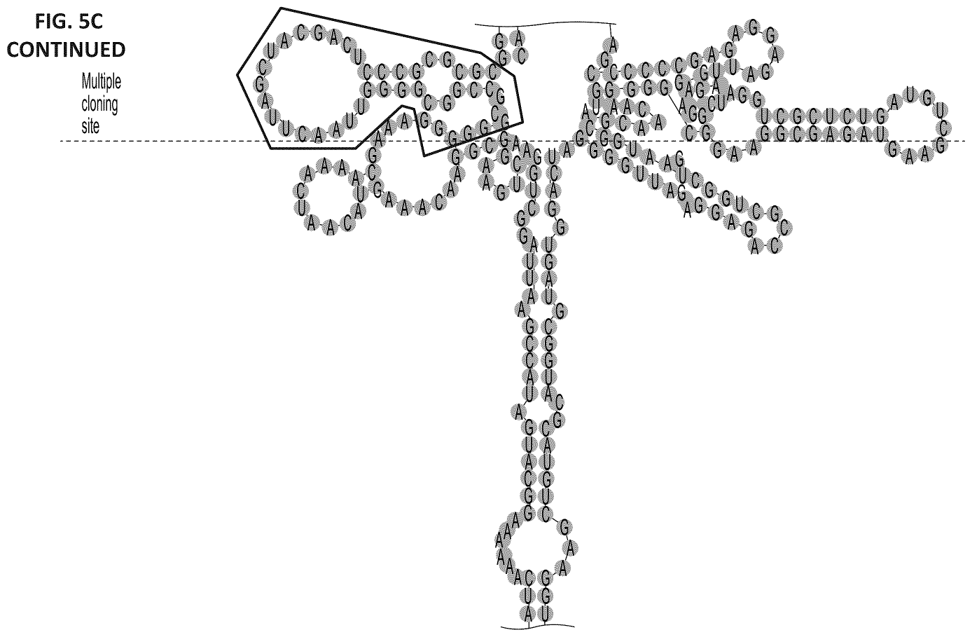

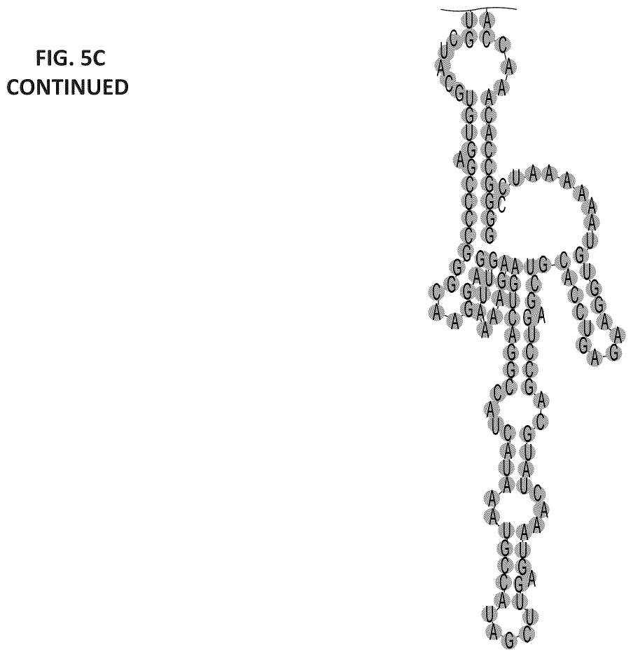

Dengue virus (DENV) is a non-segmented +ssRNA virus with 11 kb single RNA genome, which is modified at the 5′ end with a cap-1 structure for canonical cellular translation. The 3′end of the RNA genome does not carry poly(A) tail. Instead, it forms a loop structure. Interestingly, cap-independent mechanisms of translation have also been described for DENV (Mazeaud et al., 2018). Therefore, we tested both Cap1 (FIG. 5A) and Cap-less (FIG. 5B) versions of DENV RNAs (Example 6).

It is known that DENV RNA are circularized by the complementary sequences located at both 5′-UTR (including sequences downstream of an ATG initiation codon) and 3′-UTR (Mazeaud et al., 2018). To make a DENV-based synRNA construct that allows the insertion of a foreign CDS starting from an ATG to a stop codon, we introduced two mutations that change AUG to AUC in two locations (FIG. 5C). These mutations eliminate two ATG start codons upstream of a multiple cloning site. To maintain the circularization structure of a DENV-based synRNA, two corresponding mutations in 3′-UTR (CAU to GAU; GAC to CAC) were also introduced (FIG. 5C).

The final DENV-based synRNA construct (named DENVm RNA) contains 5′-Cap1, 5′-UTR (mutated), multiple cloning site (MCS), 3′-UTR (mutated), and no poly(A) (SEQ ID NO:11). The RNAs were made by IVT using T7 RNA polymerase and plasmid DNA templates linearized with a SapI restriction enzyme (Example 6). Exemplary plasmid DNA templates contained a T7 promoter; however, other promoters could be used to drive transcription (e.g., T3, SP6, etc.). Exemplary synRNAs included a 5′-Cap, which was added using CleanCap-AG (TriLink). Even so, other methods could be used to add a 5′-Cap in IVT RNA. Other Caps, such as Cap0, Cap2, etc., can also be used. As mentioned above, a Cap-less version (named Capless-DENVm RNA) was also created, which starts with GGG sequence (SEQ ID NO:12). Both natural version (unmodified, Unm) and versions modified with m1Ψ were produced. Other modified nucleosides can also be used. To test the protein production, EGFP was cloned into the MCS.

DENVm-EGFP RNA and Capless-DENVm-EGFP RNA were transfected into human fibroblasts, and expression of EGFP were monitored by microscopy and Moxi Go II cell analyzer (Examples 7, 8).

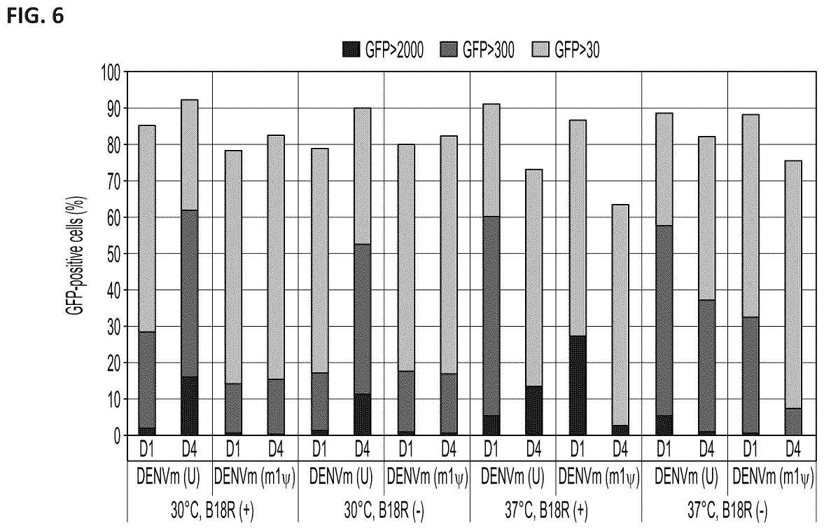

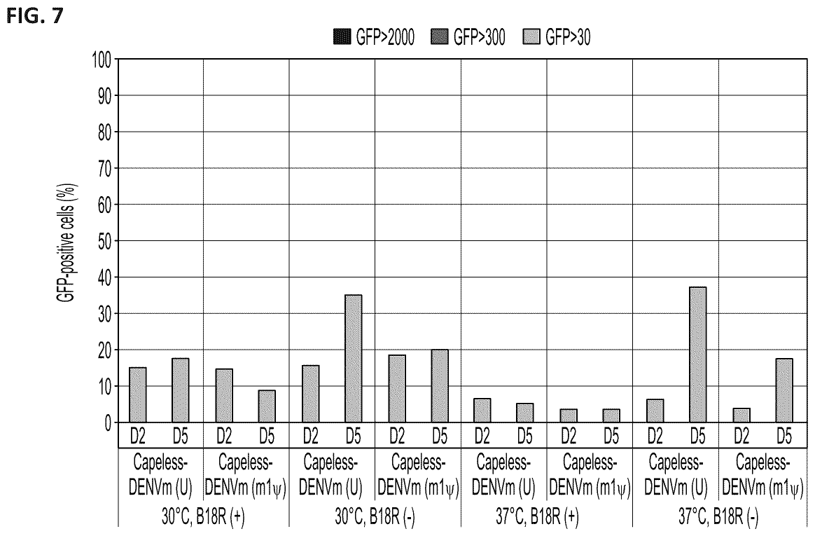

In all four culture conditions (30° C. or 37° C.; B18R+ or B18R−), DENVm synRNA was able to produce the protein—in this example, EGFP (Example 7, 8; FIG. 6). By contrast, Capless version—Capless-DENVm produced very low levels of protein (FIG. 7), suggesting that 5′-Cap is required for efficient translation from DENVm RNA.

Interestingly, DENVm synRNA has a number of unique features that are not commonly seen in usual synRNAs. First, unlike other synRNAs, DENVm synRNA shows stronger protein production in its natural form, i.e., nucleoside-unmodified, than in the nucleoside-modified form (FIG. 6). It is also notable that, unlike other synRNAs, especially unmodified form, that the protein production was not influenced by the presence or absence of B18R (FIG. 6). It is also unusual that DENVm-EGFP (unmodified) synRNA showed stronger expression on Day 4 compared to Day 1 (FIG. 6), as synRNAs in general show strong expression on Day 1, which gradually gets weaker over time. However, even in this case, Day 4 seems to be a timing for the peak expression, as the expression subsequently decreased over time and became very low by Day 11.

Accordingly, the present disclosure provides RNA molecules comprising a 5′-untranslated region (5′-UTR), at least one coding sequence (CDS) for at least one protein, and a 3′-untranslated region (3′-UTR), wherein the 5′-UTR and 3′-UTR are Dengue virus 5′-UTR and 3′-UTR. DENVm synRNA provides unique features distinct from commonly used synRNA platforms: notably, protein production from DENV2m synRNA is stronger in natural unmodified form than in modified nucleoside form. It may be particularly useful for applications that require nucleoside-unmodified synRNAs. Such examples include, but not limited to, applications for vaccines and ribonucleoproteins.

### IV. PLANT VIRUS CHROMOSOMES

Unlike +ssRNA viruses that infect insects and vertebrates, +ssRNA viruses that infect plants do not have either 5′-Cap structure nor poly(A) tails (Nicholson and White 2011). Therefore, the genetic components of these plant +ssRNA are interesting alternatives to synRNAs that have been conventionally used. However, it is essentially unknown whether the plant virus genetic components such as 5′-UTR and 3′-UTR are functional in mammalian cells, especially in human cells.

To test these plant +ssRNA viruses as platforms for the therapeutic use of synRNAs, we used the mechanism termed 3′-cap-independent translation enhancer (3′-CITE) as a guide. The 3′-CITEs are grouped into 6 major classes (Nicholson and White 2011). These 3′-CITEs are located in the 3′-UTR of +ssRNA viruses and have functions to help positioning 3′-end of the RNA near the 5′-end of the RNA. This circularization of mRNA is required for the efficient translation of mRNAs and the production of proteins. In mammalian cells, this circularization of mRNAs is mediated by the protein-protein interactions between eIF4e that binds to a 5′-Cap structure and poly(A)-binding protein that binds to a 3′-poly(A) sequences. The plant +ssRNA viruses achieve this circularization without 5′-Cap, without 3′-poly(A) sequences. We selected 5 different plant +ssRNA viruses, made synRNA constructs (Example 9, 11), and tested the EGFP expression in human fibroblast cells (Example 10, 12, 13, 14, 15).

Barley yellow dwarf virus (BYDV: NC_004750.1) contains the 3′CITE which folds into a compact cruciform RNA secondary structure and termed the BYDV-like translation element (BTE). We tested four different versions of BYDV-based synRNAs. BYDVm RNA (SEQ ID NO:13) contains 5′-Cap1, 5′-UTR, MCS, 3′-UTR (without BTE), and no poly(A). Capless-BYDVm RNA (SEQ ID NO:14) contains no 5′-Cap, 5′-UTR, MCS, 3′-UTR (without BTE), and no poly(A). These two RNAs do not contain the BTE motif. BYDV2m RNA (SEQ ID NO:15) contains 5′-Cap1, 5′-UTR, MCS, BTE (added), 3′-UTR, and no poly(A). Capless-BYDV2m RNA (SEQ ID NO:16) contains no 5′-Cap, 5′-UTR, MCS, BTE (added), 3′-UTR, and no poly(A).

Maize necrotic streak virus (MNESV: NC_007729.1) contains the 3′CITE which folds into I-shaped RNA secondary structure (ISS). We tested two different versions of MNESV-based synRNAs. MNESVm RNA (SEQ ID NO:17) contains 5′-Cap1, 5′-UTR, MCS, 3′-UTR, and no poly(A). Capless-MNESVm RNA (SEQ ID NO:18) contains no 5′-Cap, 5′-UTR, MCS, 3′-UTR, and no poly(A).

Panicum mosaic virus (PMV: U55002.1) contains the 3′CITE which folds into T-shaped RNA secondary structure and termed PMV-like translation element (PTE). We tested two different versions of PMV-based synRNAs. PMVm RNA (SEQ ID NO:19) contains 5′-Cap1, 5′-UTR, MCS, 3′-UTR, and no poly(A). Capless-PMVm RNA (SEQ ID NO:20) contains no 5′-Cap, 5′-UTR, MCS, 3′-UTR, and no poly(A).

Pea enation mosaic virus-2 (PEMV2: NC_003853.1) contains the same 3′CITE termed PTE as the PMV. We tested two different versions of PEMV2-based synRNAs. PEMV2m RNA (SEQ ID NO:21) contains 5′-Cap1, 5′-UTR, MCS, 3′-UTR, and no poly(A). Capless-PEMV2m RNA (SEQ ID NO:22) contains no 5′-Cap, 5′-UTR, MCS, 3′-UTR, and no poly(A).

Turnip crinkle virus (TCV: X05193.1) contains the 3′CITE which folds into a complex T-shaped structure (TSS), which resembles a tRNA. Because its subgenomic sequence which encodes for coat protein is more highly expressed than the genomic sequence, we used the subgenomic sequence, Turnip crinkle virus RNA for coat protein (TCV: NC_003821.3). We tested two different versions of TCV-based synRNAs. TCVm RNA (SEQ ID NO:23) contains 5′-Cap1, 5′-UTR, MCS, 3′-UTR, and no poly(A). Capless-TCVm RNA (SEQ ID NO:24) contains no 5′-Cap, 5′-UTR, MCS, 3′-UTR, and no poly(A).

First, Capless versions of plant virus-based synRNAs were tested on human fibroblast cells. These are Capless-BYDVm RNA, Capless-BYDV2m RNA, Capless-MNESVm RNA, Capless-PMVm RNA, Capless-PEMV2m RNA, and Capless-TCVm RNA. To test the protein production, EGFP was cloned into the MCS. The RNAs were made by IVT using T7 RNA polymerase and plasmid DNA templates linearized with a SapI restriction enzyme (Example 9). Exemplary plasmid DNA templates contained a T7 promoter; however, other promoters could be used to drive transcription (e.g., T3, SP6, etc.). Although naturally occurring plant virus RNAs does not seem to have any nucleoside modification, both natural version (unmodified) and versions modified with mlP were produced and tested. Other modified nucleosides can also be used.

The results showed that the translation efficiency of 5′-Cap-less +ssRNAs was rather low in human cells (Example 10).

Next, 5′-Cap1-added versions of plant virus-based synRNAs were tested on human fibroblast cells. These +ssRNA viruses do not have 5′-Cap structure in nature. These are BYDVm RNA, BYDV2m RNA, MNESVm RNA, PMVm RNA, PEMV2m RNA, and TCVm RNA. To test the protein production, EGFP was cloned into the MCS. The RNAs were made by IVT using T7 RNA polymerase and plasmid DNA templates linearized with a SapI restriction enzyme (Example 11). Exemplary plasmid DNA templates contained a T7 promoter; however, other promoters could be used to drive transcription (e.g., T3, SP6, etc.). Although naturally occurring plant virus RNAs does not seem to have any nucleoside modification, both natural version (unmodified) and versions modified with m1Ψ were produced and tested. Other modified nucleosides can also be used.

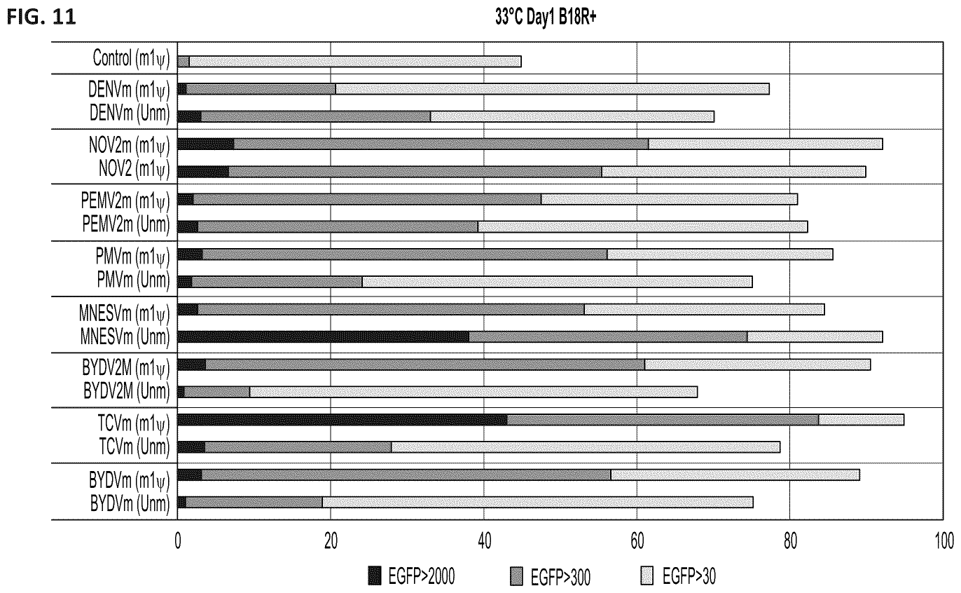

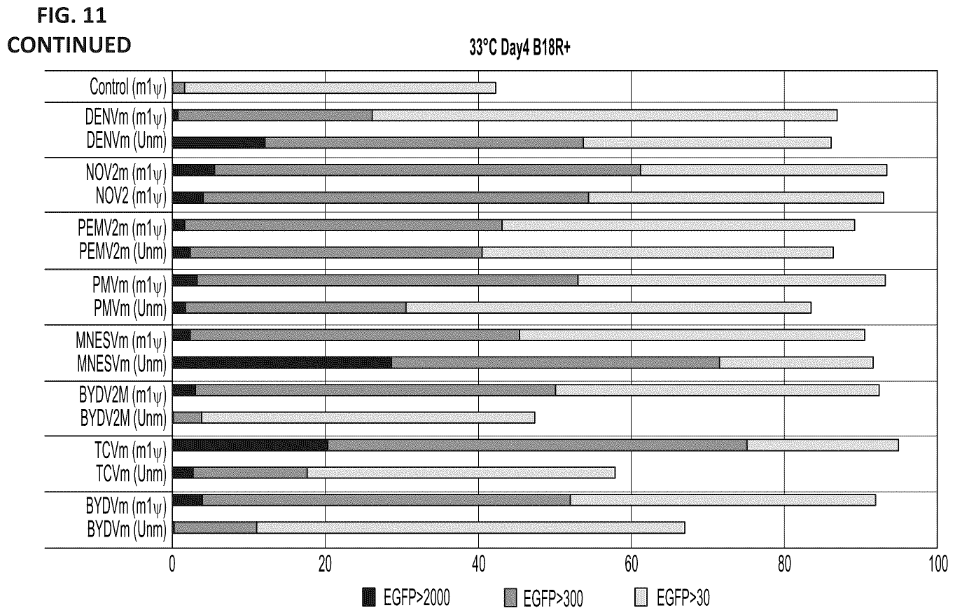

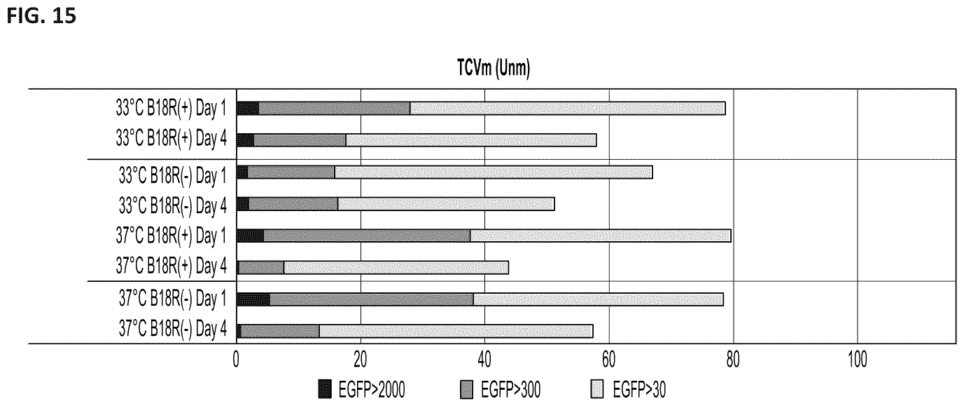

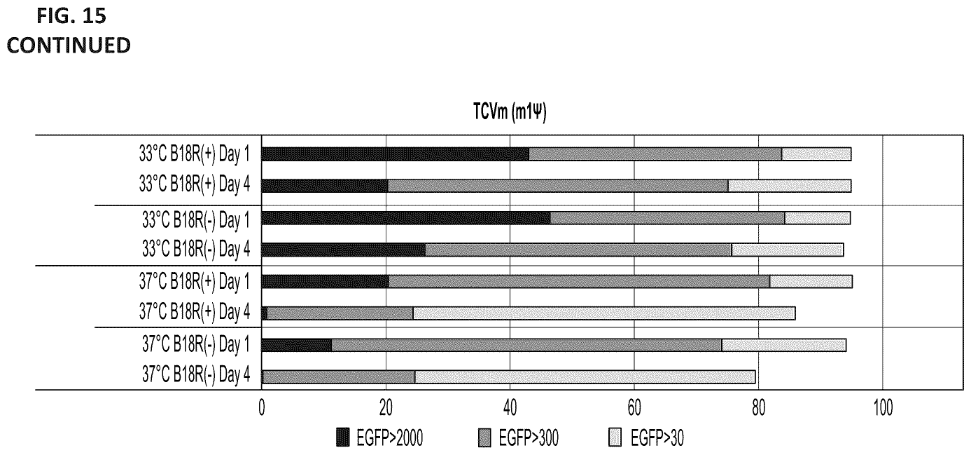

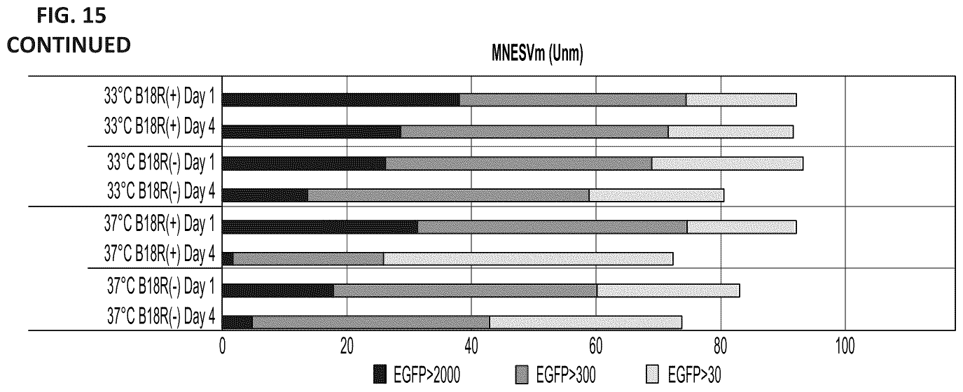

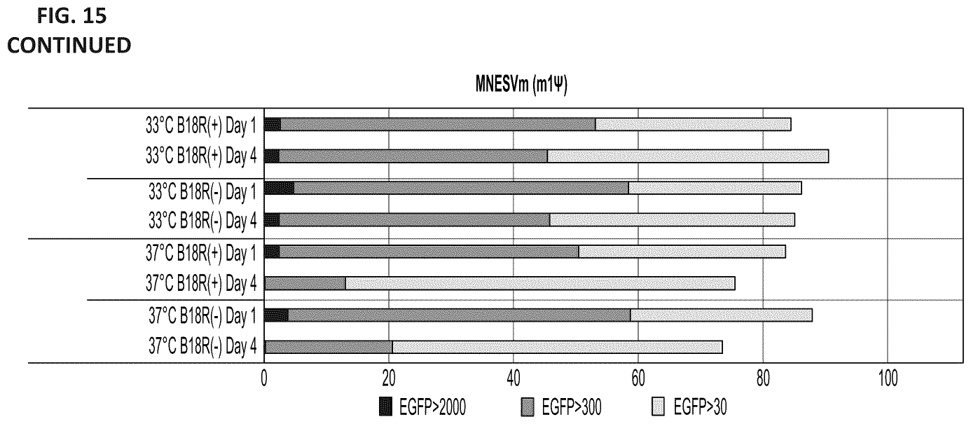

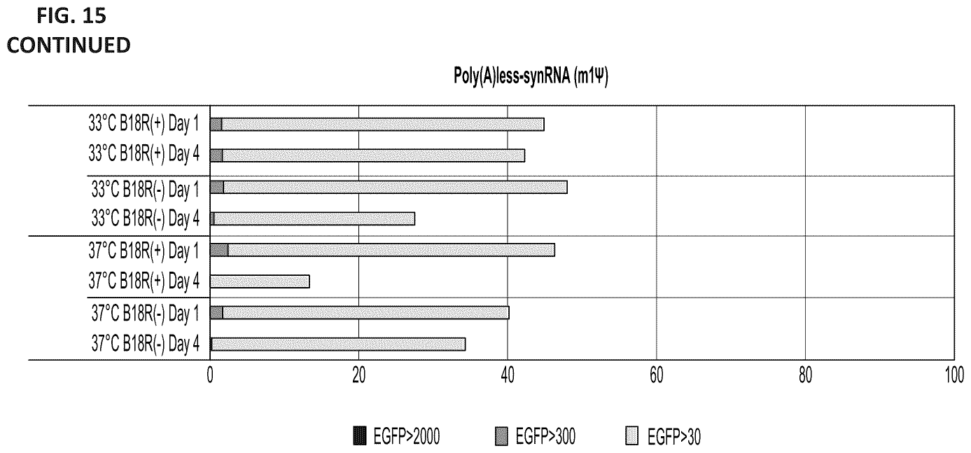

To our surprise, artificially adding 5′-Cap1 increased the translation efficiency dramatically in all four culture conditions (33° C. or 37° C.; B18R+ or B18R−) (Example 12-15; FIG. 11, 12, 13, 14). The protein expression levels were clearly higher than a poly(A)less Control-EGFP synRNA (5′-Cap1, modified with m1Ψ), which showed almost no EGFP expression (FIG. 11, 12, 13, 14). For some of these plant virus-based synRNAs, the protein expression levels were much higher than those produced from DENVm, NOV2, and NOV2m. Especially, TCVm and MNESVm are two notable examples (FIG. 15).

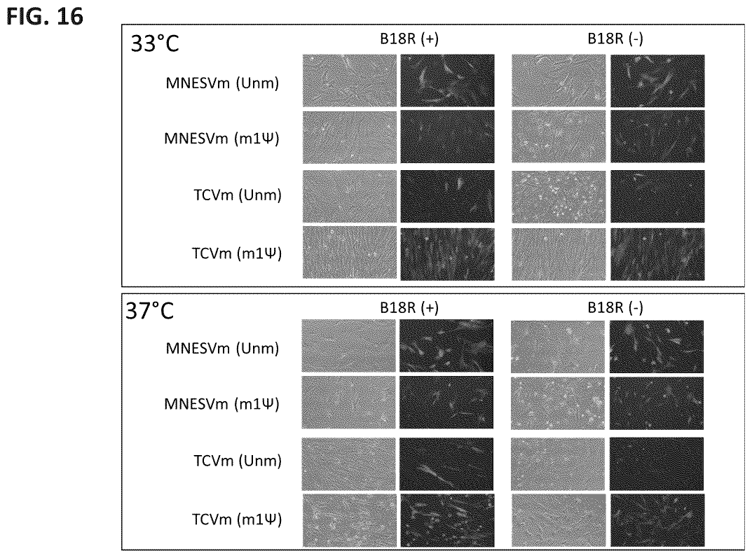

TCVm can be used as a poly(A)less version of commonly used synRNAs, because its features are similar to standard synRNAs, except for the lack of 3′-poly(A) tail: nucleoside modified version (m1Ψ) showed much higher expression of a protein than unmodified version (FIG. 15, FIG. 16); the protein expression was not influenced by the presence or absence of B18R; the expression was high on Day 1, which decreased over time, but the expression was relatively maintained till Day 4; and the expression was observed at both 33° C. and 37° C. (FIG. 15).

In contrast, MNESVm may represent a new type of synRNAs: in addition to the unique feature of poly(A) tailless, it showed much higher expression in the nucleoside unmodified form than in the nucleoside modified form (FIG. 15, FIG. 16). This expression pattern is the same as DENVm synRNA albeit different than observed with commonly used synRNAs. The expression from unmodified MNESVm was not much influenced by the presence or absence of B18R. The expression was strong on Day 1, whose expression was relatively well maintained till Day 4 (FIG. 15). These unique features are useful for the applications such as vaccines and ribonucleoproteins.

Accordingly, the present disclosure provides RNA molecules comprising a 5′-untranslated region (5′-UTR), at least one coding sequence (CDS) for at least one protein, and a 3′-untranslated region (3′-UTR), wherein the 5′-UTR and 3′-UTR are plant +ssRNA virus 5′-UTR or 3′-UTR. These new types of synRNAs lack the poly(A)-tail, and thus, provide advantages for the ease of manufacturing and CMC. They also offer high protein expression with some additional unique features, which are clearly shown in the case of TCVm and MNESVm.

### V. SYNRNA DELIVERY AND EFFICIENT PROTEIN EXPRESSION IN VIVO

In order to further test whether poly(A)less synRNAs of the present disclosure are translatable in vivo, we used commercially available lipid nanoparticles (LNP) to produce synRNA/LNP complexes. Of course, any mRNA delivery system, including, but not limited to, other LNPs, dendrimers, micelles, and polymers based on polyethylenimine, are suitable for use with the synRNAs of the present disclosure. It is also possible to deliver naked synRNAs (devoid of LNPs or other polymers) to cells in vivo. Additionally, it is possible to deliver synRNAs to cells in vivo by electroporation or by other mechanical methods.

It has been well demonstrated that synRNAs can be delivered systemically by intravenous infusion or direct injection into specific organs, by inhalation, and any other relevant methods. As a representative example, synRNAs-encoding a luciferase gene were injected into skeletal muscle.

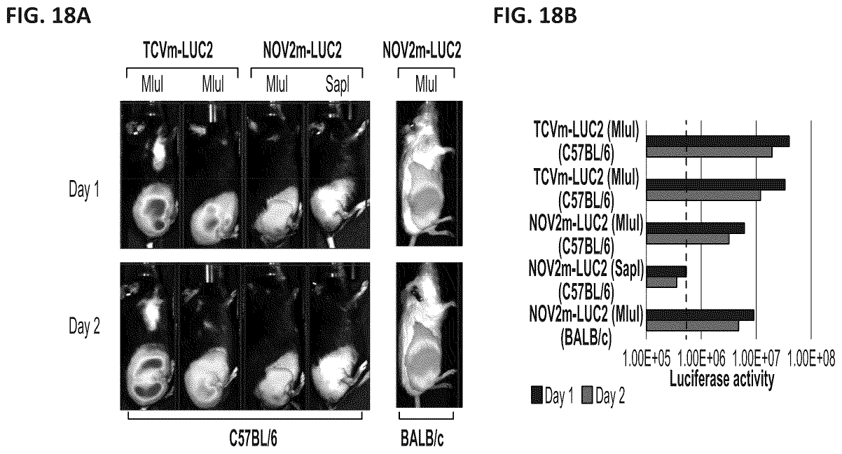

In order to develop representative poly(A)less synRNAs, TCVm and NOV2m platforms were used. Luciferase gene was cloned into NdeI-NotI site of the multiple cloning site of these vectors and synRNAs were generated by IVT after linearizing the vectors with an MluI restriction enzyme. As described in Example 14, TCVm-LUC2 (MluI) was translated at high levels in vivo, even without a poly(A) tail (FIG. 18A). NOV2m-LUC2 (MluI) also worked, although the translation efficiency was lower than that of TCVm-LUC2 (FIG. 18B). These Poly(A)less synRNAs were translatable in both C57BL/6 and BALB/c mouse strains, indicating that the Poly(A)less synRNAs are suitable for production of protein in any mouse strain. Also, considering the fact that the Poly(A)less synRNAs are translatable in human fibroblast cells in vitro, the Poly(A)less synRNAs are contemplated to be translatable in other cell and tissue types, and in other mammalian species in vitro and in vivo.

Accordingly, the present disclosure provides RNA molecules that function in vitro and in vivo without poly(A) tail.

### VI. ADDITION OF SHORT ADENINE HOMOPOLYMERS

During the manufacturing of synRNA, some purification methods use an oligo(dT) column to bind to the poly(A) tail of synRNAs (Mencin et al., 2023) If a poly(A) tail is short enough, it may be compatible with synRNA having a long coding sequence (CDS). It is, thus, conceivable to add a short adenine homopolymer (synthetic poly(A) tail) to the 3′ end of +ssRNA virus sequences described herein. However, it is not known whether unique 3′-ends of +ssRNA viruses permit the addition of adenine homopolymers as these sequences are devoid of poly(A) tails in their natural form. We, therefore, systematically tested whether poly(A)less synRNA platforms described herein are compatible with short stretches of adenine nucleotides.

We generated five sets (A0, A20, A30, A60, and A120) of synRNAs from four different constructs (Control-EGFP, NOV2m-EGFP, TCVm-EGFP, and MNESVm-EGFP). AG is the original poly(A)less synRNAs. A20 denotes the addition of 20 adenines at the 3′-end of the poly(A)less synRNAs. Likewise, A30, A60, and A120 denote the addition of 30, 60, and 120 adenines, respectively, at the 3′-end of poly(A)less synRNAs. A poly(A)-tail of about 120 adenines is a standard poly(A) tail length (Warren et al., 2010). First, the protocol of Mandal and Rossi (2013) was followed to generate DNA templates by performing tail-PCR with primers containing 0, 20, 30, 60, and 120 thymine (T) nucleotides. These DNA templates were used for IVT to produce synRNAs. Control synRNA (A120) was the same synRNA as described in previous reports (Warren et al., 2010; Mandal and Rossi, 2013), except for the presence of nucleoside modifications with m1Ψ instead of 5mC/Ψ. The 3′-UTR of the Control synRNA is hemoglobin alpha, adult chain 1 (Hba-al). NOV2m-EGFP, TCVm-EGFP, and MNESVm-EGFP are as described in the previous sections. Based on the results presented above, NOV2m-EGFP and TCVm-EGFP were modified with m1Ψ, as they performed better compared to nucleoside unmodified versions. On the other hand, MNESVm-EGFP was used in the nucleoside unmodified form (Unm), as it performed better than the nucleoside modified form (m1Ψ) in the experiments described in the previous section. All synRNAs were transfected into HDFn cells and were cultured for 24 hours at 33° C. or 37° C. with or without B18R.

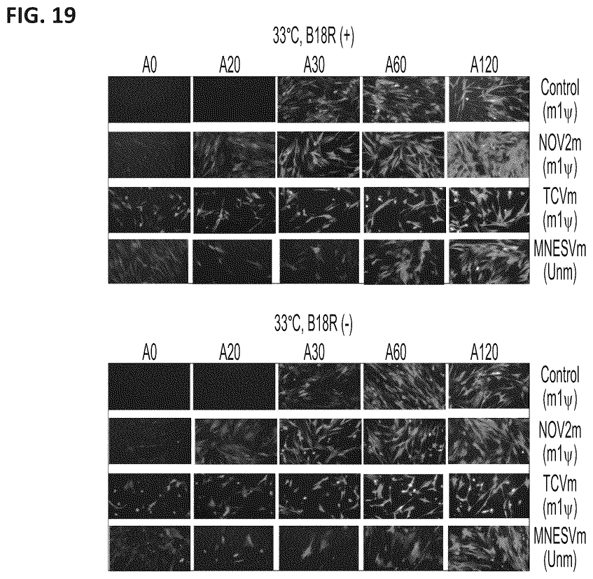

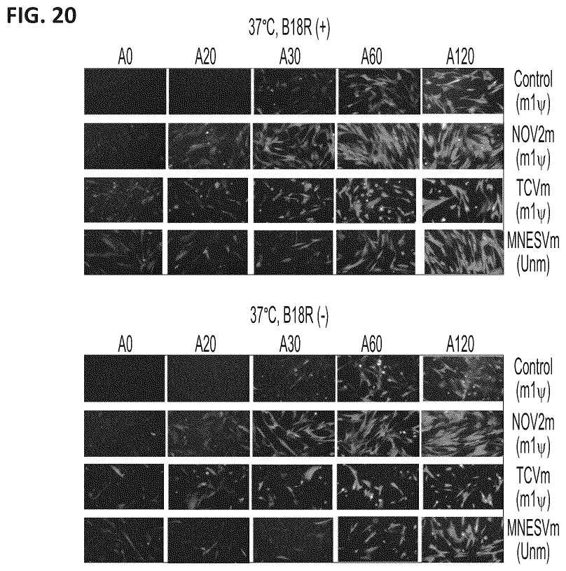

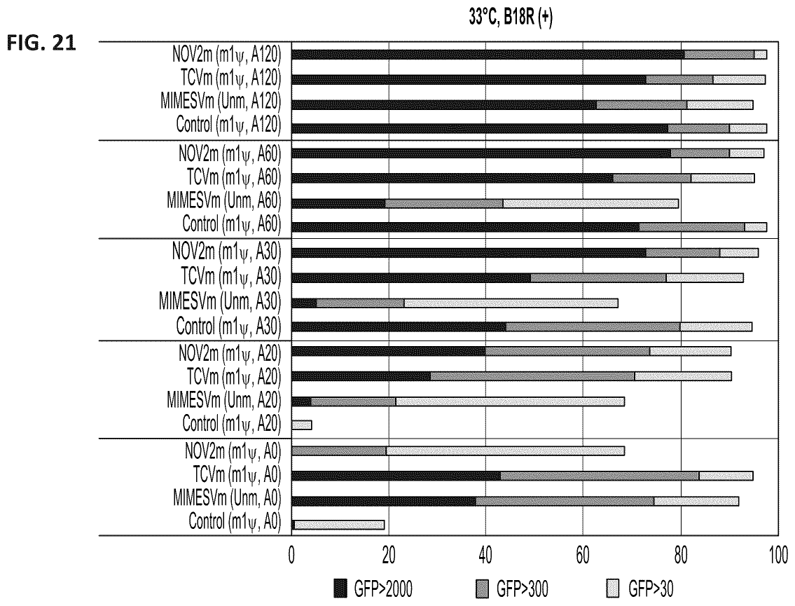

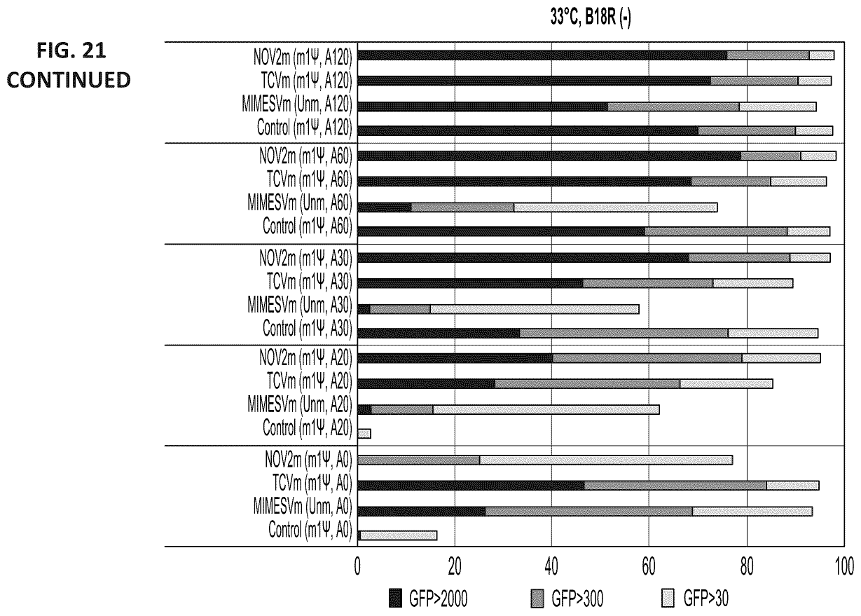

Control synRNAs showed the expression patterns as expected. AG and A20 showed no or very low translation (FIG. 19, 20, 21, 22). Starting from A30, the translation efficiency gradually increased to A60, and to A120. Control synRNAs worked well at both 33° C. and 37° C., and with and without B18R (FIG. 19, 20, 21, 22).

NOV2m synRNAs showed expression from AG (FIG. 19, 20, 21, 22). The translation efficiency increased by adding A20 homopolymer, and further increased by adding A30, A60, and A120 homopolymers. NOV2m synRNAs worked well at both 33° C. and 37° C., and with and without B18R (FIG. 19, 20, 21, 22).

TCVm synRNAs showed strong expression from AG (FIG. 19, 20, 21, 22). Interestingly, the addition of A20 homopolymer reduced the translation efficiency from A0, which increased to the level of AG only by adding an adenine homopolymer longer than A30. The translation efficiency of TCVm synRNAs was not influenced much by the temperature (33° C. or 37° C.), and by the presence or absence of B18R (FIG. 19, 20, 21, 22).

MNESVm (Unm) synRNAs showed strong expression from AG (FIG. 19, 20, 21, 22). As with TCVm synRNA, the addition of A20 and A30 homopolymers reduced the translation efficiency from A0, which increased to the level of A0 only by adding an adenine homopolymer longer than A60. The translation efficiency of MNESVm synRNAs was not influenced much by the temperature (33° C. or 37° C.), and by the presence or absence of B18R (FIG. 19, 20, 21, 22).

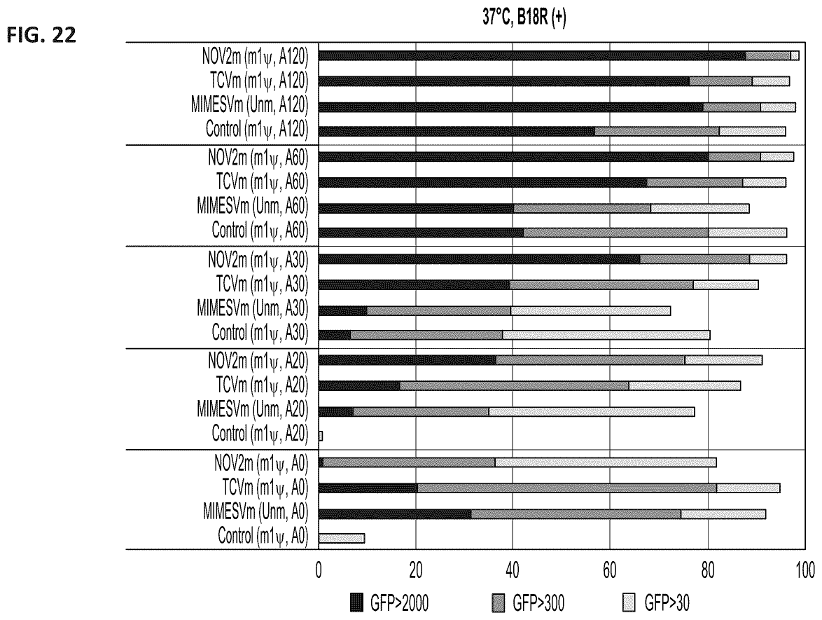

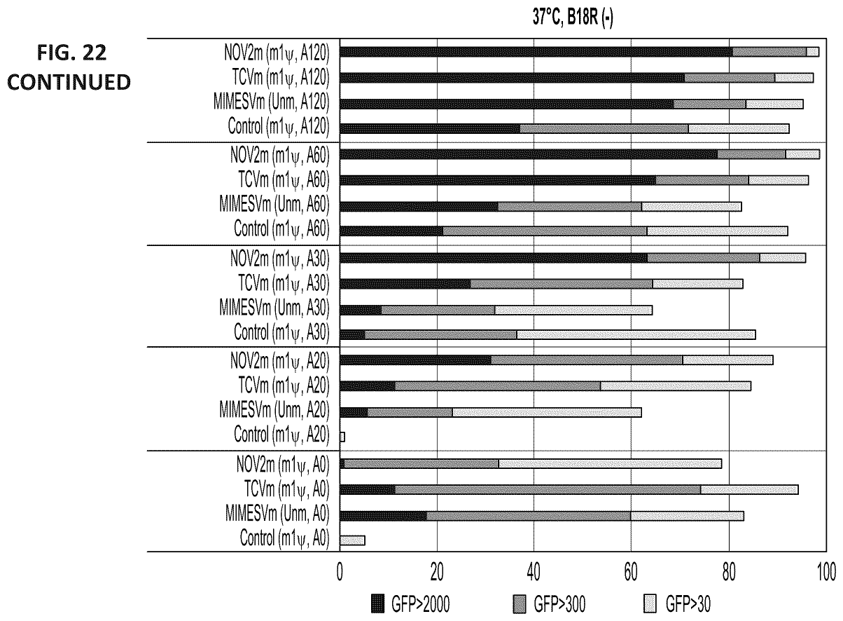

Among the four constructs tested (Control-EGFP, NOV2m-EGFP, TCVm-EGFP, and MNESVm-EGFP), NOV2m synRNAs showed the best expression levels with any length of adenine homopolymer, namely A20, A30, A60, or A120 (FIG. 19, 20, 21, 22). Notably, even with the standard A120 homopolymer, NOV2m synRNAs performed much better than Control synRNA, TCVm synRNA, and MNESVm synRNA. synRNAs are most frequently used at the natural in vivo condition (i.e., at 37° C. body temperature and in the absence of B18R). In this condition, considering the desirable short adenine homopolymer length, it is worth noting that NOV2m synRNAs with A20 and A30 showed the equal or even better expression level than the Control synRNAs with the standard A120 homopolymer (FIG. 22).

Accordingly, the present disclosure provides RNA molecules comprising a 5′-untranslated region (5′-UTR), at least one coding sequence (CDS) for at least one protein, and a 3′-untranslated region (3′-UTR), followed by a short adenine homopolymer, wherein the 5′-UTR and 3′-UTR are derived from +ssRNA viruses. These new types of synRNAs with short 3′ adenine homopolymers are suitable for expressing high levels of protein, while facilitating manufacturing and purification through use of an oligo(dT) column.

### VII. ADDITION OF SHORT ADENINE HOMOPOLYMERS TO DENVM

We also generated five sets (A0, A20, A30, A60, and A120) of synRNAs from DENVm plasmid (Example 19). A0 is the original poly(A)less synRNA. A20 denotes the addition of 20 adenines at the 3′-end of the poly(A)less synRNA. Likewise, A30, A60, and A120 denote the addition of 30, 60, and 120 adenines, respectively, at the 3′-end of poly(A)less synRNA. First, the protocol of Mandal and Rossi (2013) was followed to generate DNA templates by performing tail-PCR with primers containing 0, 20, 30, 60, and 120 thymine (T) nucleotides. These DNA templates were used for IVT to produce synRNAs. DENVm-EGFP was used in the nucleoside unmodified form (Unm), as it performed better than the nucleoside modified form (m1Ψ) in the experiments described in the previous section. All synRNAs were transfected into HDFn cells and were cultured for 24 hours at 33° C. or 37° C. with or without B18R.

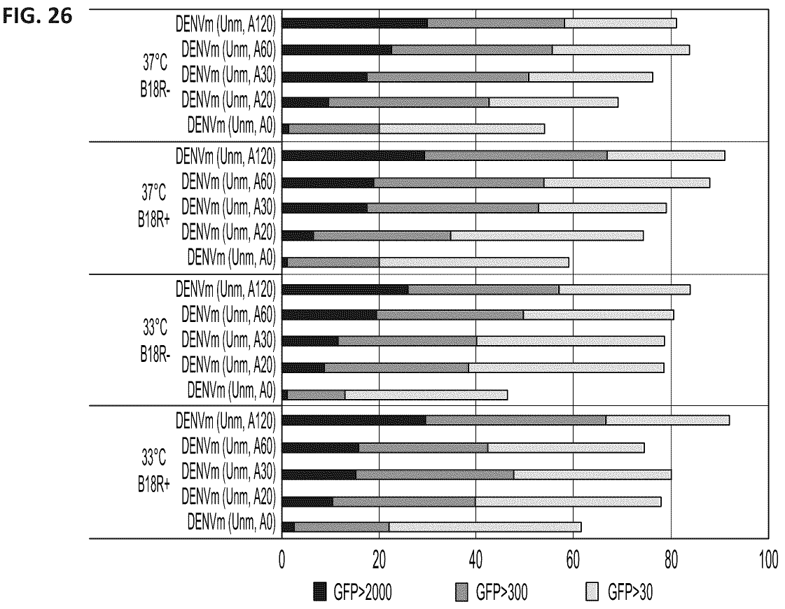

DENVm synRNAs (Umn) showed the expression from AG in vitro. The translation efficiency increased by adding A20 poly(A), and further increased by adding A30, A60, and A120. The translation efficiency of DENVm synRNAs (Unm) was not influenced very much by temperature (33° C. or 37° C.) or by the presence of B18R (FIG. 26).

Accordingly, the present disclosure provides RNA molecules comprising a 5′-untranslated region (5′-UTR), at least one coding sequence (CDS) for at least one protein, and a 3′-untranslated region (3′-UTR), followed by a short adenine homopolymer, wherein the 5′-UTR and 3′-UTR are derived from Dengue virus. Although Dengue viruses do not have polyA at the 3′-end in their natural state, the current finding demonstrates that these new types of synRNAs containing a short adenine homopolymer can be used to express high levels of a protein of interest, while providing advantages for manufacturing and purification though use of an oligo(dT) column, if necessary.

### VIII. CORONAVIRIDAE CHROMOSOMES

The genome of +ssRNA viruses in Coronaviridae is 27-32 kb in size and is the largest of all RNA virus genomes. It has a Cap structure at its 5′-end and a polyA at its 3′-end. In this sense, unlike +ssRNA virus genomes used above, the +ssRNA genome of Coronaviridae is similar to mammalian mRNAs. However, the polyA length of Coronaviridae RNA is relatively short, starting from about 45 nucleotides immediately after virus entry up to about 64 nucleotides (Wu et al., 2023).

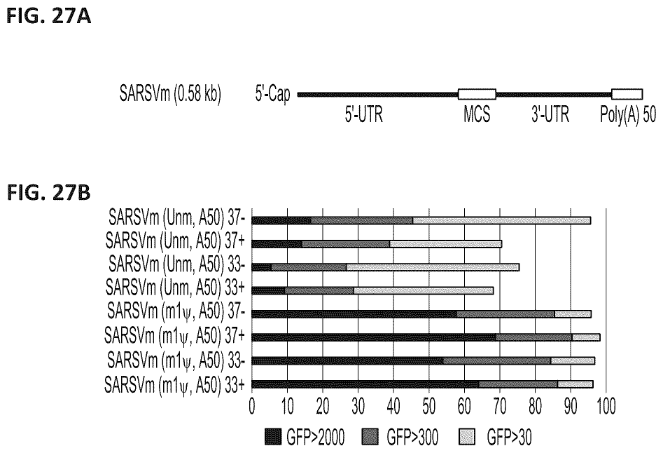

To test whether 5′-UTR, 3′-UTR, and short polyA from Coronaviridae can be used to express a heterologous protein, we constructed a plasmid vector carrying a T7 promoter, followed by DNA encoding SARSVm RNA (5′-UTR, MCS, 3′-UTR, and 50 adenines) as depicted in FIG. 27A. The 5′-UTR and 3′-UTR are taken from SARS-CoV-2 (GenBank: NC_045512.2) (Example 20). The nucleotide sequence of the 5′-UTR of SARSVm is set forth as SEQ ID NO:37, and that of the 3′-UTR and 50 adenine homopolymer is set forth as SEQ ID NO:38. After cloning EGFP into MCS, synRNA was in vitro transcribed with Cap1 (TriLink) with or without m1Ψ modification. The GFP expression from SARSVm was tested by transfecting SARSm (A50)-EGFP into HDFn cells and culturing cells at 33° C. or 37° C., with or without 250 ng/mL of B18R (Sigma) for 24 hours. As shown in FIG. 27B, SARSVm can express EGFP efficiently when mRNA is modified with m1Ψ. Interestingly, the expression is very weak when mRNA is in unmodified form, which is its natural form. This indicates that the 5′-UTR and 3′-UTR of Coronaviridae can be used as a synRNA platform, with the nucleoside-modified (i.e., artificial) form being superior.

We have also tested the addition of a longer adenine homopolymer to the natural polyA tail of SARS-CoV-2 mRNA. To this end, we have constructed a plasmid vector carrying a T7 promoter, DNA encoding SARSVm100 (5′-UTR, MCS, 3′-UTR, and 100 adenines), with the 5′-UTR and 3′-UTR taken from SARS-CoV-2 (GenBank: NC_045512.2). This mRNA is called SARSVm100. After cloning EGFP into MCS, synRNA was in vitro transcribed with Cap1 (TriLink) with or without m1Ψ modification. Compared to SARSVm, the further enhancement of EGFP expression was observed only in the m1T-modified form of SARSVm100.

Accordingly, the present disclosure provides RNA molecules comprising a 5′-untranslated region (5′-UTR), at least one coding sequence (CDS) for at least one protein, and a 3′-untranslated region (3′-UTR), followed by an adenine homopolymer, wherein the 5′-UTR and 3′-UTR are derived from +ssRNA viruses, which naturally possess adenine homopolymers at their 3′ends.

### IX. TOGAVIRIDAE CHROMOSOMES

+ssRNA genomes of Togaviridae are 9.7-11.8 kb and contain a 5′-Cap and a 3′ polyA. Togaviridae includes Alphaviruses that are widely used as self-replicating RNA (self-amplifying RNA, replicons) and include Venezuelan Equine Encephalitis Virus, Semliki Forest Virus, Sindbis Virus, and Chikungunya Virus. Natural polyA lengths of these viruses are short, typically within the range of about 25 to about 35 nucleotides.

To test whether 5′-UTR, 3′-UTR, and short polyA from Togaviridae can be used to express a heterologous protein, we have constructed a plasmid vector carrying a T7 promoter, followed by DNA encoding VEEVm RNA (5′-UTR, MCS, 3′-UTR, and 30 adenines). The 5′-UTR and 3′-UTR are taken from Venezuelan Equine Encephalitis Virus (Genbank: L014442.2). This mRNA is now called VEEVm. After cloning EGFP into MCS, synRNA was in vitro transcribed with Cap1 (CleanCap AU, TriLink) with or without m1Ψ modification. VEEVm can express EGFP efficiently when mRNA is modified with m1Ψ.

We have also tested the addition of a longer adenine homopolymer to the natural polyA tail of VEEV. To this end, we have constructed a plasmid vector carrying T7 promoter, followed by DNA encoding VEEVm100 RNA (5′-UTR, MCS, 3′-UTR, and 100 adenines) with the 5′-UTR and 3′-UTR taken from Venezuelan Equine Encephalitis Virus (Genbank: L014442.2). This mRNA is called VEEVm100. After cloning EGFP into MCS, synRNA was in vitro transcribed with Cap1 (TriLink) with or without m1Ψ modification. Compared to VEEVm, a further enhancement of EGFP expression was observed only in the m1T-modified form of VEEVm100.

### X. IN VIVO EXPRESSION OF LUCIFERASE FROM NOV2m (A50) AND DENVm (A50) IN MUSCLE

We have tested in vivo expression of +ssRNA virus-based synRNA with a focus on mRNA with additional adenine homopolymers. We have selected NOV2m (A50), NOV2m (A30), DENVm (A50), and DENVm (A30) for testing in skeletal muscle (Example 21).

For intramuscular injection, synRNAs-LUC (luciferase) were complexed with lipid nanoparticles (LNPs) Invivofectamine3.0 (ThermoFisher) according to the manufacturer's protocol. The synRNA-LUC/LNP complexes were directly injected into muscles of BALB/c mice. The expression of luciferase was monitored using a bioluminescent Imaging system.

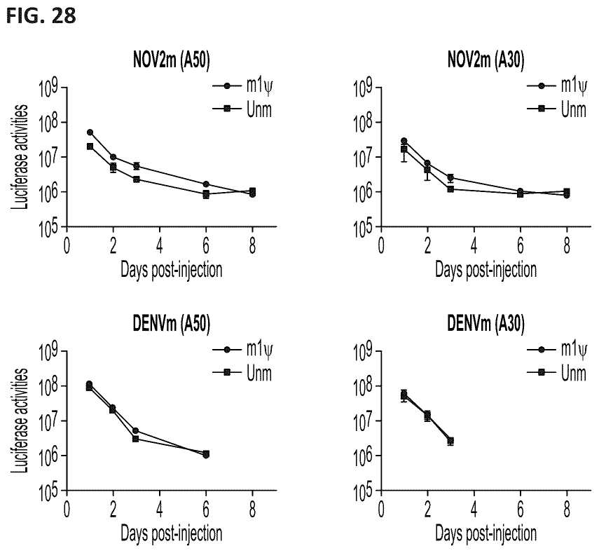

We have demonstrated that luciferase is efficiently expressed in muscle from all four of NOV2m (A50), NOV2m (A30), DENVm (A50) and DENVm (A30) mRNAs (FIG. 28). The 50 adenine homopolymer RNAs resulted in somewhat higher levels of luciferase expression than did the 30 adenine homopolymer RNAs. Nucleoside modification helped to increase the translation efficiency in muscles for NOV2m, but not for DENVm.

### XI. IN VIVO EXPRESSION OF LUCIFERASE FROM NOV2m (A50) AND DENVm (A50) IN SKIN

We have also tested in vivo expression of NOV2m (A50), NOV2m (A30), DENVm (A50), and DENVm (A30) in skin (Example 21).

For intradermal injection, we have previously demonstrated that naked (i.e., without transfection reagents nor LNPs) c-srRNA (controllable self-replicating RNA) dissolved in lactated Ringer's solution can efficiently produce proteins (see, U.S. Pat. No. 11,421,248 of Ko). We have also demonstrated that adding chitosan oligosaccharides increases protein expression (see, WO 2022/266511 and WO 2023/034881 of Elixirgen Therapeutics, Inc.). We have now assessed expression of luciferase from NOV2m and DENVm in skin after intradermal administration of naked mRNA in the presence and absence of chitosan.

About 20.0 μg of each synRNA was dissolved in 60 μL lactated Ringer's solution with or without Chitosan oligosaccharides (molecular weight ≤5 kDa, ≥75% deacetylated: Heppe Medical Chitosan GmbH: Product No. 44009). Final concentration of chitosan oligosaccharides was 1.5 μg/ml. The expression of luciferase was monitored using a bioluminescent Imaging system.

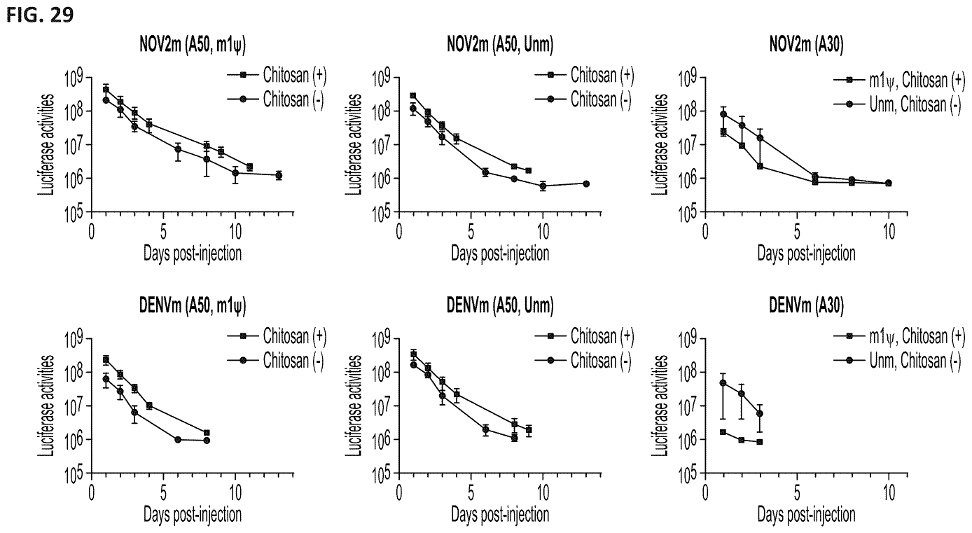

We have demonstrated that luciferase is efficiently expressed from all of NOV2m (A50), NOV2m (A30), DENVm (A50) and DENVm (A30) in skin (FIG. 29). The synRNAs with the longer 50 adenine homopolymers worked better than the synRNAs with these shorter 30 adenine homopolymers both NOV2m and DENVm. Nucleoside modification helped to increase the translation efficiency in skin for NOV2m, but not for DENVm. For DENVm, synRNA without modification (Unm) worked better than synRNA with modification (m1Ψ). We have demonstrated that overall, DENVm (A50), either modified or unmodified, performs well and slightly better than NOV2m (A50). Interestingly, administration of synRNAs in a composition comprising Chitosan oligosaccharides dramatically enhanced luciferase expression in all tested conditions: NOV2m (m1Ψ), NOV2m (Unm), DENVm (m1Ψ), and DENVm (Unm). We have demonstrated that, overall, DENVm (A50), either modified or unmodified, performs well and somewhat better than NOV2m (A50), even with nucleoside modification.

### XII. USE OF SYNRNA TECHNOLOGIES TO EXPRESS LARGE PROTEINS

The technologies disclosed herein have made it possible to produce synRNAs encoding large proteins. In general, synRNAs cannot accommodate coding sequence of large proteins. For instance, synRNA encoding the cystic fibrosis transmembrane conductance regulator (CFTR), which is 1,480 amino acids in length, is currently the largest protein that is being tested in clinical trials (Vavilis et al. 2023). Since CFTR and many other large proteins are candidates for protein replacement therapy, it is desirable to use a synRNA platform for their production.

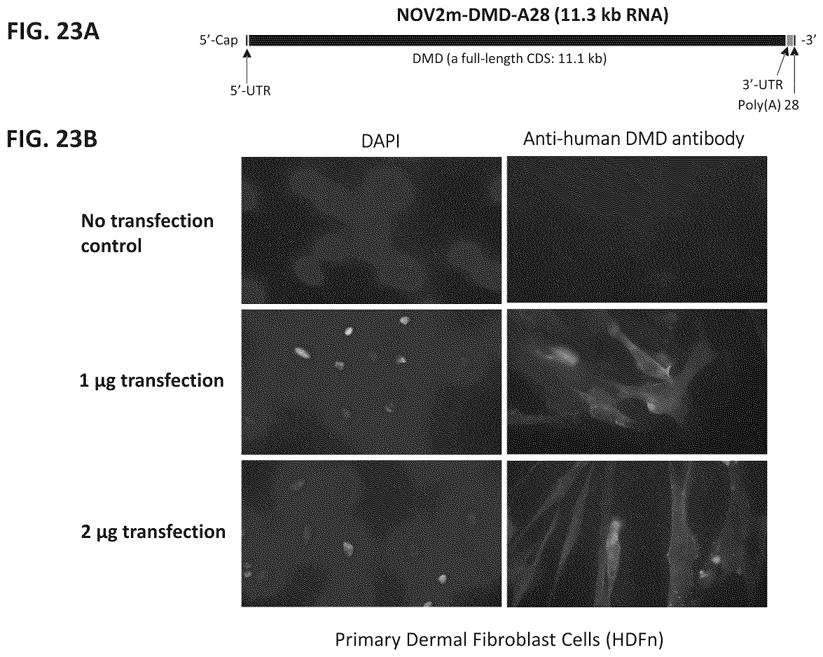

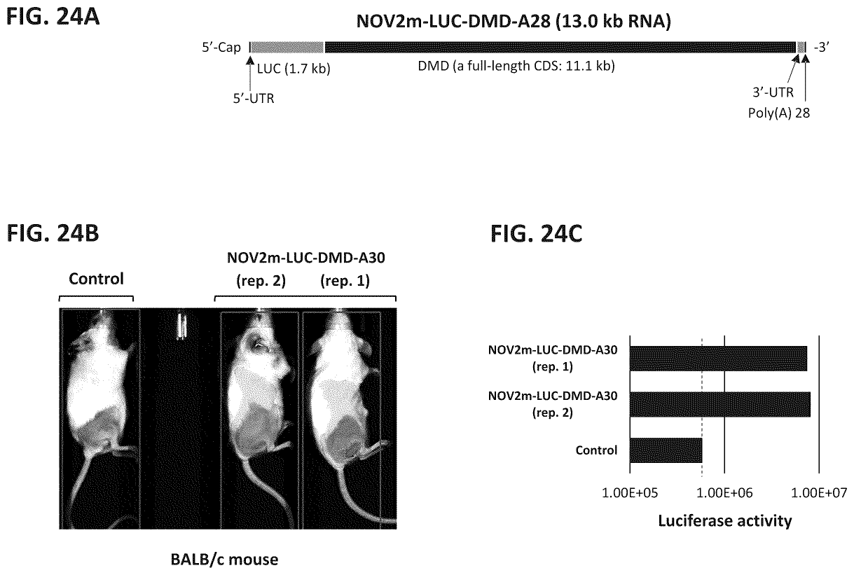

A shortage of dystrophin (DMD) protein due to a mutation causes Duchenne muscular dystrophy. The DMD protein, which is 3,685 amino acids in length, is another target for protein replacement therapy. However, due to its large protein size, current clinical trials that employ an adeno-associated virus (AAV) vector use a shortened version of DMD (micro-dystrophin). Now as a consequence of development of the present disclosure, it possible to produce a synRNA encoding a full-length human DMD protein to successfully express DMD protein in vitro (Example 16; FIG. 23A, FIG. 23B) and in vivo (Example 17: FIG. 24A, FIG. 24B, FIG. 24C). Although the examples here used the DMD sequences from the public database (e.g., NCBI), a codon optimized or truncated DMD sequences can also be used.

In the Example 17, we also used NOV2m synRNA with a 28 residue 3′-adenine homopolymer (NOV2m-A28). However, in Example 17 only, the synRNA encoded a fusion protein of a luciferase (LUC) and a full-length human dystrophin (DMD) protein (transcript variant Dp427m, NCBI accession number NM_004006). When the NOV2m-LUC-DMD-A28 synRNA of 13.0 kb length was injected into skeletal muscle, it produced a fusion protein, which was detected by in vivo luciferase assay (FIG. 24B, FIG. 24C). This indicates that a very large protein was successfully expressed in vivo from +ssRNA-virus-based synRNA.

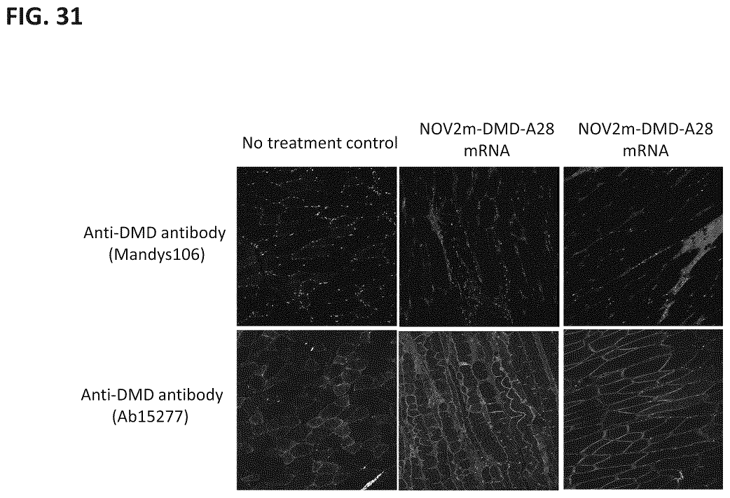

We have also demonstrated the production of human dystrophin protein in mouse skeletal muscles has by immunohistochemistry. In Example 22, we used NOV2m synRNA with a 28 residue 3′-adenine homopolymer (NOV2m-A28), but in this case, it encoded only a full-length human dystrophin protein (not a LUC-DMD fusion protein). When the NOV2m-DMD-A28 synRNA of 11.3 kb length was injected into skeletal muscle, it produced a dystrophin protein, which was detected by immunohistochemistry. Sections of muscle at the injection site were stained with anti-human DMD antibody (MANDYS106), which does not recognize mouse dystrophin but recognizes human dystrophin. The no treatment muscle did not show any staining, but the NOV2m-DMD-A28 synRNA injected muscle showed the production and proper localization of human dystrophin protein (FIG. 31, upper panels). In addition, muscle sections stained with anti-mouse DMD antibody (AB15277), which recognizes both mouse and human dystrophin proteins, showed the proper localization of mouse and human dystrophin proteins (FIG. 31, lower panels). A higher magnification image of immunostaining results from the same experiment also showed a proper localization of a full-length human dystrophin protein in mouse skeletal muscle.

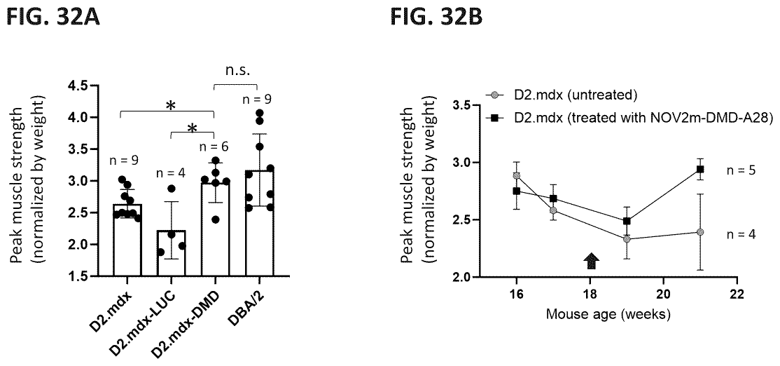

Importantly, we have now successfully demonstrated the functional recovery of skeletal muscle of mutant mice that lacks a mouse dystrophin protein as a consequence of intramuscular injection of NOV2m-DMD-A28 synRNA (Example 23). The nucleotide sequence of NOV2m-DMD-A28 synRNA is set forth as SEQ ID NO:42. The D2.mdx mouse, also known as the D2.B10-Dmd^{mdx}/J mouse, is a well-known animal model for Duchenne Muscular Dystrophy as it recapitulates human characteristics of DMD myopathology (Coley, et al. 2016, Hammers, et al. 2020). DBA/2 mice (wildtype) are often used as a control. In the Example 23, D2.mdx mutant and wild type DBA/2 mice received 3 intramuscular injections in the ventral forearm and 2 intramuscular injections in the dorsal forearm with synRNA. One week after the 6 weekly injections, the peak muscle strength of the forearm was measured by a grip strength meter. The NOV2m-DMD-A28 synRNA injected group showed statistically significant (* p<0.05) recovery of muscle strength compared to the non-injected group (D2.mdx) and a control mRNA-LUC injected group (D2.mdx-LUC) (FIG. 32A). There was no statistically significant difference between the D2.mdx-DMD and wild type DBA/2 groups. For the NOV2m-DMD-A28 synRNA injected group, there were no safety findings associated with administration of NOV2m-DMD-A28 synRNA. This result indicates that another large protein was successfully expressed in vivo from +ssRNA-virus-based synRNA.

Additionally, we have demonstrated that a single intramuscular injection of NOV2m-DMD-A28 synRNA resulted in the recovery of muscle strength in D2.mdx mutant mice (FIG. 32B). This result indicates that a single NOV2m-DMD-A28 synRNA injection not only restores muscle strength in D2.mdx mutant mice, but that the heterologous DMD proteins were stable and remained in cells at the injection site for at least 3 weeks.

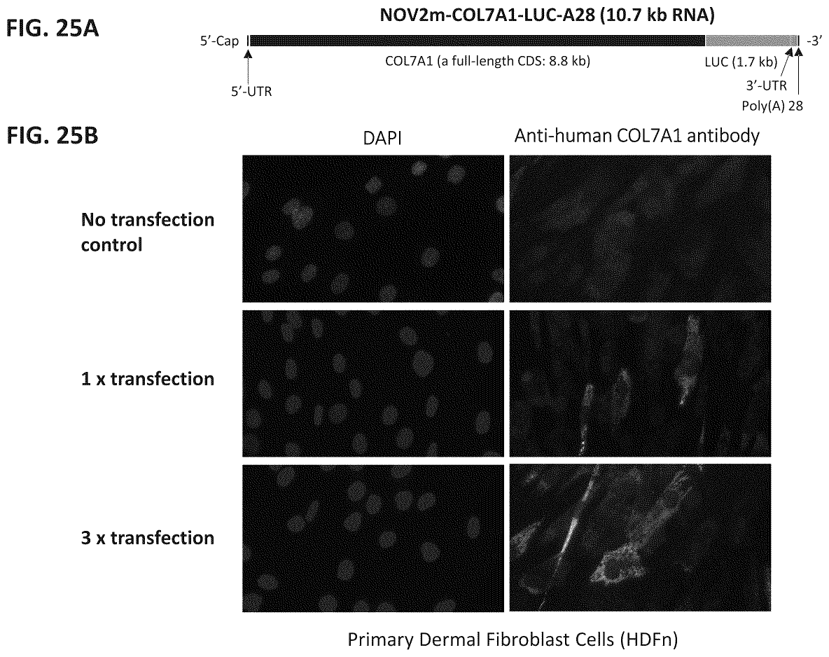

The shortage of collagen type VII alpha-1 (VII) chain (COL7A1) protein due to a mutation causes epidermolysis bullosa. Type 7 collagen is a homotrimer of COL7A1 protein, each of which are 2,944 amino acids in length. The technologies of the present disclosure have made it possible to produce a synRNA encoding a full-length human COL7A1 protein to successfully express COL7A1 protein in vitro (FIG. 25B) and in vivo. Although the examples here used the COL7A1 sequences from the public database (e.g., NCBI), codon optimized or truncated COL7A1 sequences can also be used.

The shortage of von Willebrand factor (VWF) due to a mutation causes a blood coagulation disease termed von Willebrand disease. VWF is 2,813 amino acids in length. The technologies of the present disclosure have made it possible to produce a synRNA encoding a full-length human VWF protein. Although the examples here used the VWF sequences from the public database (e.g., NCBI), codon optimized or truncated VWF sequences can also be used.

There are many diseases caused by a shortage or loss-of-function of proteins, which are potential targets for protein replacement therapies employing synRNA. On the other hand, overproduction or aberrant production of proteins causes other types of diseases. synRNAs encoding the dominant-negative form of these proteins are potential therapeutics for these diseases. One of the hurdles to applying synRNAs to such therapies is the size of proteins, as the disease-causing genes are often large proteins.

For example, according to the UniProt database, more than 200 human proteins that are longer than 2,000 amino acids in length (>6,000 nucleotide coding regions) are involved in human diseases. These genes include, but not limited to, ABCA1, ABCA12, ABCA2, ABCA4, ABCA7, ACACA, ACAN, ADGRV1, AGRN, AKAP9, ALMS1, ANK2, ANK3, ANKRD11, ANKRD17, APC, APC2, APOB, ARID1A, ARID1B, ASH1L, ASPM, ASXL3, ATM, ATR, ATRX, BDP1, BLTP1, BPTF, BRCA2, C2CD3, CACNA1A, CACNA1B, CACNA1C, CACNA1D, CACNA1E, CACNA1G, CACNA1H, CACNA1I, CAD, CCDC88C, CDH23, CELSR1, CENPE, CENPF, CEP250, CEP290, CFAP47, CHD3, CHD6, CHD7, CHD8, CIT, CNOT1, CNTRL, COL12A1, COL6A3, COL6A5, COL7A1, CPLANEl, CREBBP, CUBN, DCHS1, DMBT1, DMD, DMXL2, DNAH1, DNAH10, DNAH11, DNAH17, DNAH2, DNAH5, DNAH8, DNAH9, DNAJC13, DNHD1, DOCK3, DOCK6, DOCK7, DOCK8, DSP, DST, DYNC1H1, DYNC2H1, DYSF, EP300, EPG5, EYS, F5, F8, FANCM, FAT2, FAT4, FBN1, FBN2, FLG, FLG2, FLNA, FLNB, FLNC, FN1, FRAS1, FREM1, FREM2, FRYL, FSIP2, GPR179, HCFC1, HERC1, HERC2, HIVEP2, HMCN1, HSPG2, HTT, HUWE1, HYDIN, IGSF10, INTS1, ITPR1, ITPR2, ITPR3, KAT6A, KAT6B, KMT2A, KMT2B, KMT2C, KMT2D, KNL1, LAMA1, LAMA2, LAMA3, LAMA5, LOXHD1, LRBA, LRP1, LRP2, LRRK1, LRRK2, LYST, MACF1, MAP1B, MED12, MED12L, MED13, MED13L, MEGF8, MPDZ, MTOR, MUC5B, MXRA5, MYO15A, MYO18B, MYO7A, MYO9A, MYO9B, MYOF, NAV3, NBAS, NBEA, NBEAL2, NEB, NF1, NIN, NIPBL, NOTCH1, NOTCH2, NOTCH3, NSD1, NUP205, NUP214, OBSCN, OTOG, OTOGL, PCLO, PCM1, PCNT, PDE4DIP, PI4KA, PIEZO1, PIEZO2, PIKFYVE, PKD1, PKD1L1, PKHD1, PLCE1, PLEC, POLE, POLQ, PRKDC, PRPF8, PRR12, PTPRQ, RALGAPA1, RANBP2, RELN, RNF213, ROS1, RP1, RP1L1, RTTN, RYR1, RYR2, SACS, SCN1A, SCN2A, SCN3A, SCN5A, SETD2, SETX, SMCHD1, SNRNP200, SON, SORL1, SPAG17, SPEG, SPEN, SPG11, SPTA1, SPTAN1, SPTB, SPTBN1, SPTBN2, SPTBN4, SRCAP, SVIL, SYNE1, SYNE2, SZT2, TECTA, TENM3, TENM4, TET1, TET2, TEX15, TG, TNC, TNXB, TPR, TRIO, TRIOBP, TRPM6, TRRAP, TTN, UNC80, USH2A, USP9X, USP9Y, VCAN, VPS13A, VPS13B, VPS13C, VPS13D, VWF, WDFY3, WNK1, ZFHX2, ZFHX4, ZFYVE26, ZNF292, ZNF407, ZNF462, ZNF469.

In addition, according to the UniProt database, more than 300 human proteins that are between 1,333 amino acids and 1,999 amino acids in length (3,999 to 5,997 nucleotide coding regions) are involved in human diseases. These genes include, but not limited to, A2ML1, ABCA3, ABCC1, ABCC2, ABCC6, ABCC8, ABCC9, ADAMTS13, ADCY10, ADGRL1, AFDN, AGL, AHDC1, ALK, ALPK3, ALS2, ANAPC1, ANK1, ANKRD26, ARFGEF1, ARFGEF2, ARHGAP31, ARHGEF10, ARHGEF12, ARHGEF18, ARID2, ASXL1, ASXL2, ATP7A, ATP7B, BAZ1B, BCL9, BCOR, BCORL1, BICRA, BLM, BRCA1, BRD4, BRWD3, C2CD6, C3, C4A, C4B, C40RF54, C5, CACNA1F, CACNAlS, CAMSAP2, CAMTA1, CARMIL2, CC2D2A, CCDC88A, CDC42BPB, CDK12, CDK13, CDK5RAP2, CENPJ, CEP152, CEP164, CFAP43, CFAP44, CFAP65, CFAP74, CFTR, CHD1, CHD2, CHD4, CHD5, CIC, CLTC, CNTNAP1, CNTNAP2, COL11A1, COL11A2, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, CPAMD8, CPS1, CPSF1, CRB1, CUL7, CUX1, CUX2, DCC, DEPDC5, DICERI, DLEC1, DNMBP, DNMT1, DOCK2, DUOX2, EIF2AK4, EIF4G1, ELP1, EPRS1, ERBB3, ERCC6, ERCC6L2, EXPH5, FANCA, FANCD2, FHOD3, FLT1, FLT4, FMN2, FOCAD, FYCO1, GEMIN5, GLI2, GLI3, GREB1L, GRIN2A, GRIN2B, GRIN2D, HECW2, HFM1, IFT140, IFT172, IGF1R, IGSF1, INSR, IQSEC2, ITGB4, KANK1, KATNIP, KDM3B, KDM5A, KDM5B, KDM5C, KDM6A, KDM6B, KDR, KIAA0586, KIAA1549, KIDINS220, KIF14, KIF15, KIF1A, KIF1B, KIF21A, KIF26A, KIF7, KMT2E, LAMA4, LAMB1, LAMB2, LAMC3, LCT, LRP4, LRP5, LRP6, LRPPRC, LTBP1, LTBP2, LTBP4, MADD, MAGI2, MAP3K1, MAPK8IP3, MAPKBP1, MAST1, MBD5, MCM3AP, MED23, MET, MIA2, MIA3, MLH3, MSH6, MST1R, MYH10, MYH11, MYH14, MYH2, MYH3, MYH6, MYH7, MYH8, MYH9, MYLK, MYO3A, MYO5A, MYO5B, NALCN, NCAPD2, NCAPD3, NCOA1, NCOA2, NEXMIF, NFASC, NHS, NLRP1, NPHP3, NPHP4, NRXN1, NSD2, NSD3, NUP155, NUP160, NUP188, NUP98, OBSL1, OTOF, PALLD, PAPPA2, PARD3, PBRM1, PCDH11Y, PCDH15, PHIP, PIK3C2A, PLCH1, PLEKHG2, PLXNA1, PNPLA6, POGZ, POLA1, POLR1A, POLR2A, POLR3A, PRG4, PRX, PTCH1, PTPN23, PTPRF, PXDN, QRICH2, RAB3GAP2, RAIl, RAPGEF2, RERE, RIC1, RIMS1, RIMS2, ROBO2, ROBO3, RUSC2, SAMD9, SAMD9L, SBF1, SBF2, SCAPER, SCN10A, SCN11A, SCN4A, SCN8A, SCN9A, SCRIB, SETBP1, SETD1A, SETD1B, SETD5, SHANK2, SHANK3, SHOC1, SHROOM4, SI, SIPA1L3, SKIC3, SLX4, SMARCA2, SMARCA4, SOS1, SOS2, SPEF2, STRC, SYCP2, SYNGAPI, SYNJ1, TAF1, TANC2, TCF20, TCHH, TCOF1, TDRD9, TECPR2, TET3, TEX14, THADA, THOC2, THSD7A, TIAM1, TMEM94, TNIK, TNR, TNRC6A, TNRC6B, TOGARAMI, TONSL, TOP2B, TP53BP1, TRIP11, TRIP12, TRPM1, TRPM3, TRPM7, TSC2, TUBGCP6, UBR1, USP6, WDR19, WDR62, WDR81, WRN, XDH, YEATS2, ZMYM2, ZMYM3, ZNF142, ZNF335, ZNFX1.

### XIII. USE OF SYNRNA TECHNOLOGIES TO EXPRESS LARGE FUSION PROTEINS OR MULTIPLE PROTEIN SUBUNITS

The technologies disclosed herein have made it possible to produce synRNAs encoding large fusion proteins (FIG. 24, FIG. 25). Proteins often function as a complex, which forms within a cell, of multiple protein subunits. Therefore, it is imperative that every protein subunit of the protein complex be expressed within the same cell. If the subunit proteins are encoded by separate synRNAs, they may not be delivered to the same cell. Thus, it is desirable to encode each protein subunit in the same synRNA. This can be achieved by making a single fusion protein, in which each protein coding region is fused via 2A self-cleaving peptides. Alternatively, coding sequences of protein subunits can be connected via an internal ribosome entry site (IRES). Regardless of the connection element utilized, the size of synRNAs encoding fusion proteins or multiple protein subunits will be large. The technologies of the present disclosure are suitable for use with lengthy coding regions (e.g., a large cargo space) to permit production of fusion proteins and polyprotein complexes within host cells.

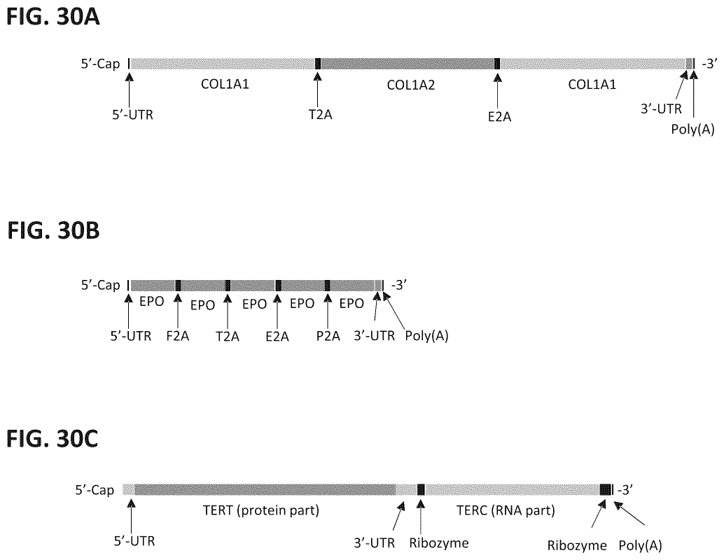

One example of a large polyprotein complex is type I collagen, which accounts for 70% of the total collagen found in the human body. The shortage of type I collagen due to mutations causes a disease known as osteogenesis imperfecta. Type I collagen is a hetero trimer of two COL1A1 proteins (1,464 amino acids in length) and one COL1A2 protein (1,366 amino acids in length). To achieve the 2:1 stoichiometry, two COL1A1 coding sequences and one COL1A2 coding sequence are fused via nucleic acids encoding 2A self-cleaving peptides to resulting in a synRNA of greater than 13 kb in length, which encodes a fusion protein of 4,338 amino acids in length) (FIG. 30A). Although such a construct is a major challenge for conventional RNA technology, the synRNAs of the present disclosure (without a poly(A) tail or with a short 3′ adenine homopolymer) facilitate expression of this large fusion protein from a single synRNA molecule.

Some fusion proteins may consist of multiple copies of the same protein. This increases the number of proteins that to be produced from a single synRNA. Alternatively, multiple copies of a coding region of the same protein can be linked via an IRES in a single synRNA. One such example is erythropoietin (EPO), which is 193 amino acids in length. The technologies of the present disclosure (e.g., synRNAs without poly(A) tail or synRNAs with a short 3′ adenine homopolymer) facilitate expression of multiple EPO proteins in a single synRNA, resulting in the production of more EPO proteins in vivo from a single synRNA (FIG. 30B).

### XIV. USE OF SYNRNA TECHNOLOGIES TO EXPRESS RIBONUCLEOPROTEINS

Ribonucleoproteins function by forming a complex with a protein and its partner RNA. One example of a ribonucleoprotein is telomerase, which is composed of telomerase reverse transcriptase (TERT) as the protein component, and telomerase RNA (TERC) as the RNA component. Other examples are CRISPR/Cas9 genome editing tools, which are composed ofCas9 as the protein component, and a single guide RNA (sgRNA) as the RNA component. Other genome editing tools, such as Cas12a, Cas13, and “prime editing” are also based on ribonucleoproteins. Ribonucleoproteins also include, but are not limited to, ribosome, vault ribonucleoproteins, RNase P, hnRNP, and small nuclear RNPs (snRNPs).

Therapeutic application of ribonucleoproteins requires the delivery of a protein and an RNA in the same cell. As a protein can be expressed in a cell by introduction of synRNA, it is desirable to encode both a protein and an RNA in the same synRNA. The RNA component can be cleaved out by flanking it with self-cleaving ribozyme RNA sequences or target sequences of ribozymes such as RNase III, RNase P, RNase Z, or Cas proteins such as Cas12a. However, the cleavage of synRNAs will separate the 5′-Cap and/or the 3′-poly(A) from the coding sequence of the synRNA, resulting in the loss of the protein production.

This problem is solved by the technologies of the present disclosure (synRNAs without poly(A) tail or synRNAs with a short 3′ adenine homopolymer). A typical design of such synRNAs consists of 5′-Cap, 5′-UTR, CDS (encoding protein part), 3′-UTR, ribozyme(s), RNA part, ribozyme(s), no or a short adenine homopolymer. For example, a single synRNA encoding both TERT (protein part) and TERC (RNA part) (FIG. 30C). After delivery of the synRNA to a cell, ribozymes cleave this synRNA, producing two RNA molecules: a protein production part (5′-Cap, 5′-UTR, CDS, 3′-UTR), and an RNA part. Although the protein production part does not have a poly(A) tail, it is still suitable for production of a protein, which is bound to an RNA part to form a protein/RNA complex (ribonucleoprotein). The RNA part includes, but is not limited to, TERC, sgRNA, crRNA, microRNA, and shRNA. The technologies of the present disclosure provide synRNAs with a large cargo space, which can accommodate both coding sequences of large proteins and large RNAs (or repeats of RNAs).

### XV. ENUMERATED EMBODIMENTS

1. An RNA molecule comprising a 5′-untranslated region (5′-UTR), at least one coding sequence (CDS) for at least one protein, and a 3′-untranslated region (3′-UTR), wherein
   the 3′-UTR is a Nodamura virus RNA2 (NOV2) 3′-UTR or fragment thereof, or a Nodamura virus RNA1 (NOV1) 3′-UTR or fragment thereof, wherein the fragment is at least 40 nucleotides in length, and
   the CDS is heterologous to NOV2 or NOV1, and replaces an open reading frame of a Nodamura virus capsid protein of NOV2 or an open reading frame of a Nodamura virus RNA-dependent RNA polymerase (RdRp).
2. The RNA molecule of embodiment 1, wherein the 3′-UTR is a NOV2 3′-UTR.
3. The RNA molecule of embodiment 2, wherein the 3′-UTR comprises the nucleotide sequence of SEQ ID NO:10, or a nucleotide sequence having at least 90%, 95%, 96%, 97%, 98% or 99% identity to SEQ ID NO:10.
4. The RNA molecule of embodiment 1, wherein the 3′-UTR is a NOV1 3′-UTR.
5. The RNA molecule of embodiment 4, wherein the 3′-UTR comprises the nucleotide sequence of SEQ ID NO:7, or a nucleotide sequence having at least 90%, 95%, 96%, 97%, 98% or 99% identity to SEQ ID NO:7.
6. The RNA molecule of embodiment 1, comprising:
   (i) the nucleotide sequence of SEQ ID NO:6 as the 5′-UTR and the nucleotide sequence of SEQ ID NO:7 as the 3′-UTR; or
   (i) the nucleotide sequence of SEQ ID NO:9 as the 5′-UTR and the nucleotide sequence of SEQ ID NO:10 as the 3′-UTR.
7. An RNA molecule comprising a 5′-untranslated region (5′-UTR), at least one coding sequence (CDS) for at least one protein, and a 3′-untranslated region (3′-UTR), wherein
   the 3′-UTR comprises a virus 3′-UTR or fragment thereof, wherein the fragment is at least 40 nucleotides in length,
   the CDS is heterologous to the virus, and replaces at least a portion of an open reading frame of the virus, and
   the virus is a positive-sense, single-stranded RNA (+ssRNA) virus whose genome lacks a poly(A) tail.
8. The RNA molecule of embodiment 7, wherein the virus is a member of a viral family selected from the group consisting of Nodaviridae, Flaviridae, and Tetraviridae.
9. The RNA molecule of embodiment 8, wherein the virus is a member of the Nodaviridae family.
10. The RNA molecule of embodiment 9, wherein the virus is a Nodamuravirus or a flock house virus.
11. The RNA molecule of embodiment 8, wherein the virus is a member of the Flaviridae family.
12. The RNA molecule of embodiment 11, wherein the virus is a Dengue virus.
13. The RNA molecule of embodiment 7, wherein the virus is a plant virus.
14. The RNA molecule of embodiment 13, wherein the plant virus is a Barley yellow dwarf virus (BYDV), a Maize necrotic streak virus (MNESV), a Panicum mosaic virus (PMV), a Pea enation mosaic virus-2 (PEMV2), or a Turnip crinkle virus (TCV).
15. The RNA molecule of any one of embodiments 1-14, comprising at least one modified nucleoside.
16. The RNA molecule of embodiment 15, wherein the at least one modified nucleoside comprises “5mC+Ψ”, “m1Ψ”, “5moU” or “V”, optionally wherein the at least one modified nucleoside comprises “m1Ψ”, optionally wherein the at least one modified nucleoside comprises “5mC+Ψ”.
17. The RNA molecule of any one of embodiments 1-16, wherein the at least one CDS comprises two or more CDSs for two or more distinct proteins.
18. The RNA molecule of embodiment 17, wherein the two or more CDS are operably linked to form a fusion protein comprising the two or more distinct proteins.
19. The RNA molecule of embodiment 17, wherein the two or more CDS are separated from each other by an internal ribosome entry site (IRES).
20. The RNA molecule of embodiment 17, wherein the two or more CDS are separated from each other by nucleotides encoding a flexible linker or 2A self-cleaving peptide.
21. The RNA molecule of any one of embodiments 1-20, wherein the RNA molecule comprises a heterologous adenine homopolymer at its 3′ end that is no more than about 60 nucleotides in length, optionally wherein the heterologous adenine homopolymer is from 20 to 60 nucleotides in length.
22. The RNA molecule of any one of embodiments 1-20, wherein the at least one protein is a full-length human dystrophin or a full-length human collagen type VII alpha-1 (VII) chain (COL7A1) protein.
23. A DNA template for the RNA molecule of any one of embodiments 1-22, optionally wherein a first restriction enzyme site or sites is present between the between the 5′-UTR and the at least one coding region, and a second restriction enzyme site or sites is present between the at least one coding region and the 3′-UTR.
24. A plasmid comprising the DNA template of embodiment 23, wherein the plasmid comprises a promoter upstream of the 5′UTR.
25. A host cell comprising a plasmid of embodiment 24.
26. A recombinant virus comprising the RNA molecule of any one of embodiments 1-22.
27. A method for expressing a protein, comprising contacting a mammalian cell with the RNA molecule of any one of embodiments 1-22.
28. The method of embodiment 27, wherein the contacting is in vitro.
29. The method of embodiment 27, wherein the contacting is in vivo.
30. The method of any one of embodiments 27-29, wherein the contacting is done in the presence of a B18R protein.
31. An RNA molecule comprising from 5′ to 3′, a 5′-untranslated region (5′-UTR), at least one coding sequence (CDS) for at least one protein, a 3′-untranslated region (3′-UTR), and a homopolymer of adenine, wherein
   the 3′-UTR comprises a virus 3′-UTR or a fragment thereof, wherein the fragment is at least 40 nucleotides in length,
   the CDS and the homopolymer of adenine are heterologous to the virus, and
   the virus is a positive-sense, single-stranded RNA (+ssRNA) virus whose genome lacks a poly(A) tail.
32. The RNA molecule of embodiment 31, wherein the adenine homopolymer is no more than about 60 nucleotides in length.
33. The RNA molecule of embodiment 32, wherein the adenine homopolymer is from 20 to 60 nucleotides in length.
34. The RNA molecule of embodiment 31, wherein the virus is a member of a viral family selected from the group consisting of Nodaviridae, Flaviridae, and Tetraviridae.
35. The RNA molecule of embodiment 31, wherein the virus is a member of the Nodaviridae family.
36. The RNA molecule of embodiment 35, wherein the virus is a Nodamuravirus or a flock house virus.
37. The RNA molecule of embodiment 31, wherein the virus is a member of the Flaviridae family.
38. The RNA molecule of embodiment 37, wherein the virus is a Dengue virus.
39. The RNA molecule of embodiment 31, wherein the virus is a plant virus.
40. The RNA molecule of embodiment 39, wherein the plant virus is a Barley yellow dwarf virus (BYDV), a Maize necrotic streak virus (MNESV), a Panicum mosaic virus (PMV), a Pea enation mosaic virus-2 (PEMV2), or a Turnip crinkle virus (TCV).
41. The RNA molecule of any one of embodiments 31-40, comprising a least one modified nucleoside.
42. The RNA molecule of embodiment 41, wherein the at least one modified nucleoside comprises “5mC+Ψ”, “m1Ψ”, “5moU” or “Ψ”, optionally wherein the at least one modified nucleoside comprises “m1Ψ”, optionally wherein the at least one modified nucleoside comprises “5mC+Ψ”.
43. The RNA molecule of any one of embodiments 31-42, wherein the at least one protein is a full-length human dystrophin or a full-length human collagen type VII alpha-1 (VII) chain (COL7A1) protein.
44. The RNA molecule of embodiment 31, wherein the adenine homopolymer is between about 60 nucleotides and about 120 nucleotides in length.
45. An RNA molecule comprising from 5′ to 3′: a 5′-untranslated region (5′-UTR), at least one coding sequence (CDS) for at least one protein, and a 3′-untranslated region (3′-UTR), wherein
   the 3′-UTR comprises a virus 3′-UTR or a fragment thereof, wherein the fragment is at least 40 nucleotides in length,
   the CDS is heterologous to the virus and replaces at least an open reading frame of an RNA-dependent RNA polymerase of the virus, and
   the virus is a positive-sense, single-stranded RNA (+ssRNA) virus.
46. The RNA molecule of embodiment 45, wherein the RNA molecule further comprises an adenine homopolymer of between 15 and 200 nucleotides in length downstream of the 3′-UTR.
47. The RNA molecule of embodiment 46, wherein the virus is a member of the Coronaviridae family.
48. The RNA molecule of embodiment 47, wherein the virus is a severe acute respiratory syndrome coronavirus-2 (SARS-CoV-2).
49. An RNA molecule comprising from 5′ to 3′: a 5′-untranslated region (5′-UTR), at least one coding sequence (CDS) for at least one protein, and a 3′-untranslated region (3′-UTR), wherein
   the 3′-UTR comprises a virus 3′-UTR or fragment thereof, wherein the fragment is at least 40 nucleotides in length,
   the CDS encodes a full-length human dystrophin or a full-length human collagen type VII alpha-1 (VII) chain (COL7A1) protein, and replaces at least a portion of an open reading frame of the virus, and
   the virus is a positive-sense, single-stranded RNA (+ssRNA) virus.
50. The RNA molecule of embodiment 49, wherein the genome of the virus lacks a poly(A) tail.
51. The RNA molecule of embodiment 50, further comprising a heterologous adenine homopolymer at its 3′ end, optionally wherein the adenine homopolymer is from 20 to 60 nucleotides in length.
52. The RNA molecule of embodiment 49, wherein the genome of the virus comprises a poly(A) tail, and the RNA molecule further comprises the poly(A) tail.
53. The RNA molecule of embodiment 52, further comprising a heterologous adenine homopolymer at the 3′ end of the poly(A) tail, optionally wherein the poly(A) tail and the heterologous adenine homopolymer together are between 20 and 120 nucleotides in length.
54. An RNA molecule comprising from 5′ to 3′: a 5′-untranslated region (5′-UTR), at least one coding sequence (CDS) for at least one protein, and a 3′-untranslated region (3′-UTR), wherein
   the 5′-UTR is a 5′-UTR of a Dengue virus or fragment thereof, and the 3′-UTR is a 3′-UTR of the Dengue virus or fragment thereof, wherein the fragment is at least 40 nucleotides in length and
   the CDS is heterologous to the Dengue virus and replaces an open reading frame of the Dengue virus such that a portion of the open reading frame that interacts with a complementary sequence of the 3′-UTR to form a circular conformation remains in the RNA molecule.
55. The RNA molecule of embodiment 54, wherein a first start codon in the portion of the open reading frame remaining in the RNA molecule is mutated so as not to start translation of a first corresponding genome sequence, and a first corresponding portion of the 3′-UTR that interacts with the first start codon to form the circular conformation is mutated so that the mutated first start codon remains complementary to the mutated first corresponding portion of the 3′-UTR to form the circular conformation.
56. The RNA molecule of embodiment 55, wherein a second start codon in the portion of the open reading frame remaining in the RNA molecule is mutated so as not to start translation of a second corresponding genome sequence, and a second corresponding portion of the 3′-UTR that interacts with the second start codon to form the circular conformation is mutated so that the mutated second start codon remains complementary to the mutated second corresponding portion of the 3′-UTR to form the circular conformation.
57. The RNA molecule of any one of embodiments 54-56, further comprising a 5′-cap.
58. The RNA molecule of any one of embodiments 54-57, wherein:
   (i) the 5′-UTR comprises the nucleotide sequence of SEQ ID NO:25 or a nucleotide sequence having at least 90%, 95%, 96%, 97%, 98% or 99% identity to SEQ ID NO:25; and
   (ii) the 3′-UTR comprises the nucleotide sequence of SEQ ID NO:26 or a nucleotide sequence having at least 90%, 95%, 96%, 97%, 98% or 99% identity to SEQ ID NO:26.
59. The RNA molecule of one of embodiments 54-58, further comprising a homopolymer of adenine downstream of the 3′-UTR.
60. The RNA molecule of embodiment 59, wherein the adenine homopolymer is between about 30 and about 60 nucleotides in length.
61. An RNA molecule comprising from 5′ to 3′: a 5′-untranslated region (5′-UTR), at least one coding sequence (CDS) for at least one protein, and a 3′-untranslated region (3′-UTR), wherein
   the 5′-UTR is a 5′-UTR of a plant virus or fragment thereof, and the 3′-UTR is a 3′-UTR of a plant virus or fragment thereof, wherein the fragment is at least 40 nucleotides in length,
   the CDS is heterologous to the plant virus, and replaces at least a portion of an open reading frame of the plant virus,
   the virus is a positive-sense, single-stranded RNA (+ssRNA) virus whose genome lacks a poly(A) tail, and
   the 3-UTR comprises a 3′-cap-independent translation enhancer (3′-CITE).
62. The RNA molecule of embodiment 61, further comprising a 5′-cap that is heterologous to the plant virus.
63. The RNA molecule of embodiment 61 or embodiment 62, further comprising a homopolymer of adenine that is heterologous to the plant virus.
64. The RNA molecule of embodiment 63, wherein the adenine homopolymer is between about 30 and about 60 nucleotides in length.
65. The RNA molecule of one of embodiments 61-64, wherein the plant virus is a Barley yellow dwarf virus (BYDV), a Maize necrotic streak virus (MNESV), a Panicum mosaic virus (PMV), a Pea enation mosaic virus-2 (PEMV2), or a Turnip crinkle virus (TCV).
66. The RNA molecule of one of embodiments 61-64, wherein the 3′-CITE is a BYDV-like translation element (BTE), a PMV-like translation element (PTE), an I-shaped secondary structure (ISS) or a T-shaped structure (TSS).
67. The RNA molecule of embodiment 66, wherein the 3′-CITE is a BTE.
68. The RNA molecule of embodiment 67, wherein:
   (i) the 5′-UTR comprises the nucleotide sequence of SEQ ID NO:27 or a nucleotide sequence having at least 90%, 95%, 96%, 97%, 98% or 99% identity to SEQ ID NO:27; and (ii) the 3′-UTR comprises the nucleotide sequence of SEQ ID NO:28 or a nucleotide sequence having at least 90%, 95%, 96%, 97%, 98% or 99% identity to SEQ ID NO:28. 69. The RNA molecule of embodiment 66, wherein the 3′-CITE is a PTE.
70. The RNA molecule of embodiment 69, wherein:
   (i) the 5′-UTR comprises the nucleotide sequence of SEQ ID NO:29 or a nucleotide sequence having at least 90%, 95%, 96%, 97%, 98% or 99% identity to SEQ ID NO:29; and
   (ii) the 3′-UTR comprises the nucleotide sequence of SEQ ID NO:30 or a nucleotide sequence having at least 90%, 95%, 96%, 97%, 98% or 99% identity to SEQ ID NO:30.
71. The RNA molecule of embodiment 69, wherein:
   (i) the 5′-UTR comprises the nucleotide sequence of SEQ ID NO:31 or a nucleotide sequence having at least 90%, 95%, 96%, 97%, 98% or 99% identity to SEQ ID NO:31; and
   (ii) the 3′-UTR comprises the nucleotide sequence of SEQ ID NO:32 or a nucleotide sequence having at least 90%, 95%, 96%, 97%, 98% or 99% identity to SEQ ID NO:32.
72. The RNA molecule of embodiment 66, wherein the 3′-CITE is an ISS.
73. The RNA molecule of embodiment 72, wherein:
   (i) the 5′-UTR comprises the nucleotide sequence of SEQ ID NO:33 or a nucleotide sequence having at least 90%, 95%, 96%, 97%, 98% or 99% identity to SEQ ID NO:33; and
   (ii) the 3′-UTR comprises the nucleotide sequence of SEQ ID NO:34 or a nucleotide sequence having at least 90%, 95%, 96%, 97%, 98% or 99% identity to SEQ ID NO:34.
74. The RNA molecule of embodiment 66, wherein the 3′-CITE is TSS.
75. The RNA molecule of embodiment 74, wherein:
   (i) the 5′-UTR comprises the nucleotide sequence of SEQ ID NO:35 or a nucleotide sequence having at least 90%, 95%, 96%, 97%, 98% or 99% identity to SEQ ID NO:35; and
   (ii) the 3′-UTR comprises the nucleotide sequence of SEQ ID NO:36 or a nucleotide sequence having at least 90%, 95%, 96%, 97%, 98% or 99% identity to SEQ ID NO:36.
76. An RNA molecule comprising from 5′ to 3′: a 5′-untranslated region (5′-UTR), at least one coding sequence (CDS) for at least one protein, and a 3′-untranslated region (3′-UTR), wherein
the 5′-UTR is a 5′-UTR of virus or fragment thereof, and the 3′-UTR is a 3′-UTR of the virus or fragment thereof, the fragment being at least 40 nucleotides in length, the CDS is heterologous to the virus and replaces an open reading frame of the virus, and the virus is a positive-sense, single-stranded RNA (+ssRNA) virus.
77. The RNA molecule of embodiment 76, wherein the virus 3′-UTR comprises a heterologous adenine homopolymer downstream of the homologous adenine homopolymer of the virus.
78. The RNA molecule of embodiment 77, wherein:
   (i) the 5′-UTR comprises the nucleotide sequence of SEQ ID NO:37 or a nucleotide sequence having at least 90%, 95%, 96%, 97%, 98% or 99% identity to SEQ ID NO:37; and
   (ii) the 3′-UTR comprises the nucleotide sequence of SEQ ID NO:38 or a nucleotide sequence having at least 90%, 95%, 96%, 97%, 98% or 99% identity to SEQ ID NO:38.
79. The RNA molecule of embodiment 76, wherein the at least one protein comprises is a full-length human dystrophin or a full-length human collagen type VII alpha-1 (VII) chain (COL7A1).
80. The RNA molecule of embodiment 76, wherein the CDS comprises the nucleotide sequence of SEQ ID NO:39 or a nucleotide sequence having at least 90%, 95%, 96%, 97%, 98% or 99% identity to SEQ ID NO:39.
81. The RNA molecule of embodiment 76, wherein the CDS comprises the nucleotide sequence of SEQ ID NO:40 or a nucleotide sequence having at least 90%, 95%, 96%, 97%, 98% or 99% identity to SEQ ID NO:40.
82. The RNA molecule of embodiment 76, comprising the nucleotide sequence of SEQ ID NO:41 or a nucleotide sequence having at least 90%, 95%, 96%, 97%, 98% or 99% identity to SEQ ID NO:41.
83. The RNA molecule of any one of embodiments 76-82, wherein the 5′-UTR and the 3′-UTR form a circular conformation.
84. An RNA molecule comprising from 5′ to 3′: a virus 5′-untranslated region (5′-UTR), a multiple cloning site (MCS), and a virus 3′-untranslated region (3′-UTR), wherein
   the virus is a positive-sense, single-stranded RNA (+ssRNA) virus, and the MCS is from 18 to 60 nucleotides in length.
85. The RNA molecule of embodiment 84, wherein the MCS comprises the nucleotide sequence of SEQ ID NO:8.
86. The RNA molecule of embodiment 84, comprising the nucleotide sequence of SEQ ID NO:3 or a nucleotide sequence having at least 90%, 95%, 96%, 97%, 98% or 99% identity to SEQ ID NO:3.
87. The RNA molecule of embodiment 84, comprising a nucleotide sequence selected from SEQ ID NOS:11-24 or a nucleotide sequence having at least 90%, 95%, 96%, 97%, 98% or 99% identity to a nucleotide sequence selected from SEQ ID NOS:11-24.
88. The RNA molecule of any one of embodiments 84-87, further comprising at least one coding sequence (CDS) for at least one protein located within the MCS or replacing a portion of the MCS.
89. A DNA template for the RNA molecule of any one of embodiments 31-88.
90. The DNA template of embodiment 89, wherein a first restriction enzyme site or sites is present between the between the 5′-UTR and the at least one coding region, and a second restriction enzyme site or sites is present between the at least one coding region and the 3′-UTR.
91. A plasmid comprising the DNA template of embodiment 90, wherein the plasmid comprises a promoter upstream of the 5′UTR.
92. A host cell comprising a plasmid of embodiment 91.
93. A recombinant virus comprising the RNA molecule of any one of embodiments 31-88.
94. A method for expressing a protein, comprising contacting a mammalian cell with the RNA molecule of any one of embodiments 31-88.
95. The method of embodiment 94, wherein the contacting is in vitro.
96. The method of embodiment 94, wherein the contacting is in vivo.
97. The method of any one of embodiments 94-96, wherein the contacting is done in the presence of a B18R protein.

### EXAMPLES

Abbreviations: +ssRNA (positive-sense single-stranded RNA); 3′-UTR (3′-untranslated region); 5′-UTR (5′-untranslated region); 5mC+Ψ(5-methylcytosine and pseudouridine); 5moU (5-methoxyuridine); B18R (vaccinia virus-encoded receptor protein); BTE (BYDV-like translation element); BYDV (Barley yellow dwarf virus); CDS (coding sequence); COLlA (collagen type I alpha chain); COL7A1 (collagen type VII alpha-1 chain); DAPI (4′,6-diamidino-2-phenylindole); DENV (dengue virus); DMD (dystrophin); EGFP (enhanced green fluorescent protein); EPO (erythropoietin); GOI (gene of interest); GS-linker (Glycine-Serine Linker); HDFn (human dermal fibroblast, neonate); IRES (internal ribosome entry site); IVT (in vitro transcription); kb (kilobase); LUC (luciferase); mlI (N1-methylpseudouridine); MCS (multiple cloning site); MNESV (Maize necrotic streak virus); NOV (Nodamura virus); NOV RNA1 (NOV1); NOV RNA2 (NOV2); ORF (open reading frame); PCR (polymerase chain reaction); PEMV2 (Pea enation mosaic virus-2); PMV (Panicum mosaic virus); PTE (PMV-like translation element); Ψ(pseudouridine); synRNA (synthetic mRNA); RdRp (RNA-dependent RNA polymerase); TCV (Turnip crinkle virus); and Unm (unmodified).

### Example 1. Production of NOV1-EGFP, NOV2-EGFP, NOV2m, and NOV2m-EGFP synRNAs

This example describes the construction of plasmid DNAs and their use for production of synRNAs. Nodamura virus (NOV) contains a bipartite +ssRNA genome, RNA1 (NOV1) and RNA2 (NOV2). Both RNA1 and RNA2 have a 5′-Cap, but do not have poly(A) tail at their 3′ends. RNA1 encodes RNA-dependent RNA polymerase (RdRp), and RNA2 encodes a capsid protein. As proof of concept, open reading frames of NOV1 and NOV2 were replaced with the open reading frame (ORF) of a gene of interest (GOI).

### Materials and Methods

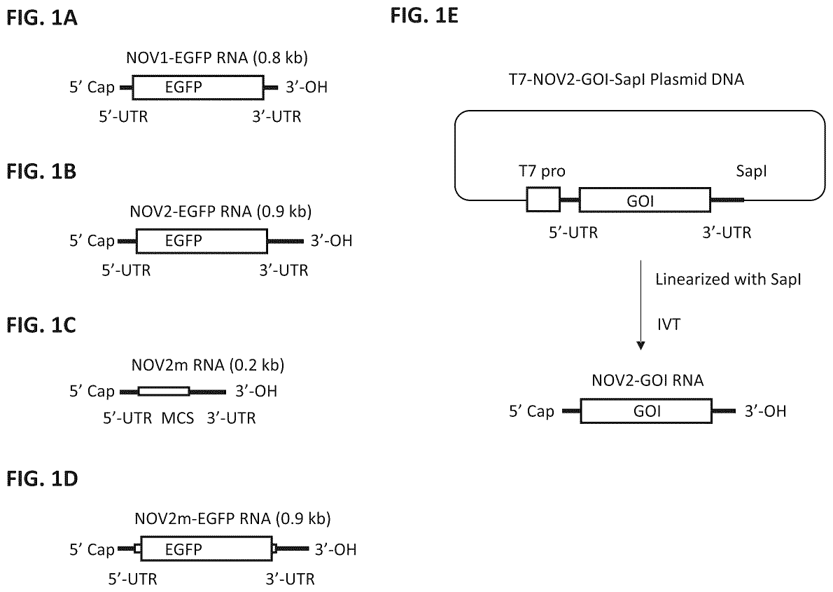

Design of NOV RNAs and construction of template plasmid DNAs. NOV1 RNA genome sequence (NCBI Accession: NC_002690) and NOV2 RNA genome sequence genome 2 (NOV2, NCBI Accession: NC_002691) were used as starting sequences. Open reading frames (from Start codon to Stop Codon) of NOV1 and NOV2 were replaced with the coding sequence of enhanced green fluorescent protein (EGFP) (FIG. 1A, FIG. 1B). To facilitate the cloning of a variety of genes in the NOV2 expression cassette of plasmid DNA, the ORF of NOV2 was replaced with multiple cloning site sequence. This expression cassette was named NOV2m (FIG. 1C). NOV2m-EGFP was produced by cloning EGFP with Kozak consensus sequences into AscI-NotI sites (FIG. 1D). An exemplary plasmid DNA for production of NOV1-GOI mRNA, NOV2-GOI mRNA, and NOV2m-GOI mRNA is shown in FIG. 1E. The T7 RNA polymerase promoter sequence was added to the 5′end of NOV1 and NOV2 to facilitate in vitro transcription (IVT). A 5′-Cap can be added to mRNA using standard methods. However, for convenience, CleanCap AG (Henderson 2021; TriLink) was used to add a 5′-Cap (Cap1). This was made possible by insertion of nucleotide (A) immediately downstream of the T7 promoter. At the 3′end, a SapI restriction enzyme site was added to produce the same 3′end sequence as is present in the NOV1 and NOV2 RNA fragments.

Production of synthetic RNA by in vitro transcription. The plasmid DNAs were linearized with the SapI restriction enzyme and used as a template DNA for in vitro transcription (IVT), which was carried using MEGAscript T7 Kit (ThermoFisher Scientific), according to the manufacturer's instructions. A 5′-Cap was incorporated using CleanCap AG (TriLink). RNAs with modified nucleosides were produced according to the manufacturer's instructions. Five versions of RNAs were prepared: a standard RNA without any nucleoside modifications (Unmodified), RNA modified with 5-methylcytosine and pseudouridine (5mC+Ψ), RNA modified with N1-methylpseudouridine (m1Ψ), RNA modified with 5-methoxyuridine (5moU), RNA modified with pseudouridine (Ψ).

### Results

Schematic diagrams of synRNAs that were successfully produced are shown in FIG. 1A, FIG. 1B, FIG. 1C and FIG. 1D. The RNA sequences of these constructs are set forth as SEQ ID NOs:1-4.

### Example 2. EGFP Protein Expression from NOV1 mRNA and NOV2 mRNA

This example describes the transfection of human fibroblast cells with synRNAs in which the open reading frames (from Start codon to Stop Codon) of NOV1 and NOV2 were replaced with the coding sequence of enhanced green fluorescent protein (EGFP). Viability of transfected cells, as well as expression of EGFP protein from transfected cells is also described.

### Materials and Methods

Cell Culture. Human Dermal Fibroblasts, neonatal (HDFn) were purchased from ThermoFisher Scientific (Catalog No. C0045C) and cultured according to the manufacturer's instructions.

Transfection of NOV1-EGFP synRNA and NOV2-EGFP synRNA into HDFn. 3×10\hat{ }4 HDFn cells/well were plated in 24-well plate at day −1. The following day, cells were transfected with 0.5 μg of synRNA using the MessengerMax transfection reagent (ThermoFisher). Four different synRNAs were used: NOV1-EGFP synRNA (Unmodified), NOV1-EGFP synRNA (modified with 5mC and Ψ), NOV2-EGFP synRNA (Unmodified), and NOV2-EGFP synRNA (modified with 5mC and Ψ). HDFn cells were cultured at 37° C. or 33° C., with or without 250 ng/mL of recombinant B18R (Sigma). B18R is a vaccinia virus protein that functions as a decoy receptor for type I interferons. B18R binds human interferon-alpha and increases cell viability during RNA transfection.

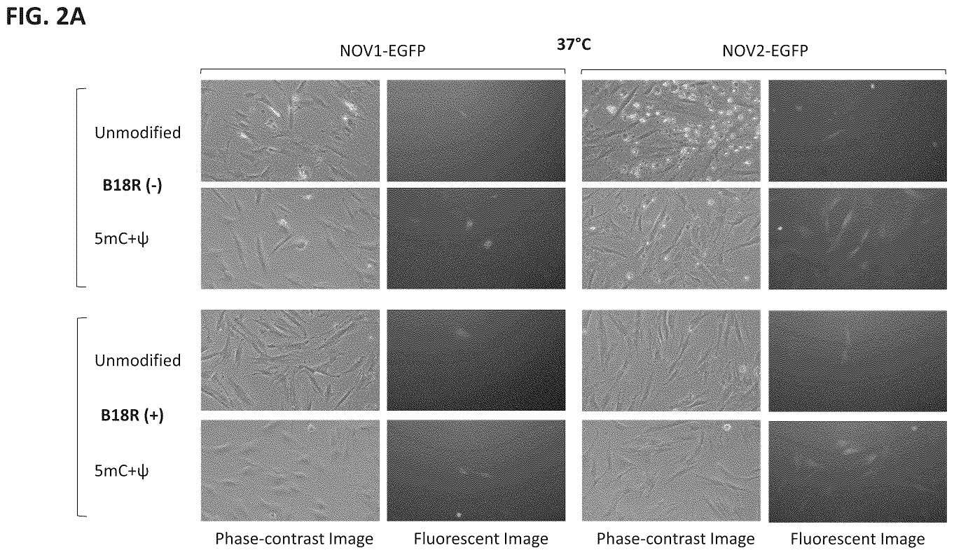

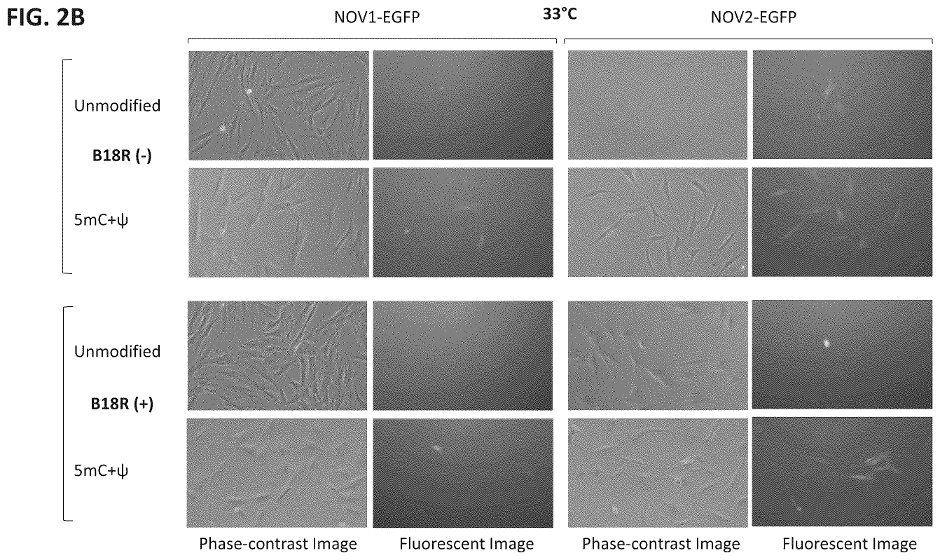

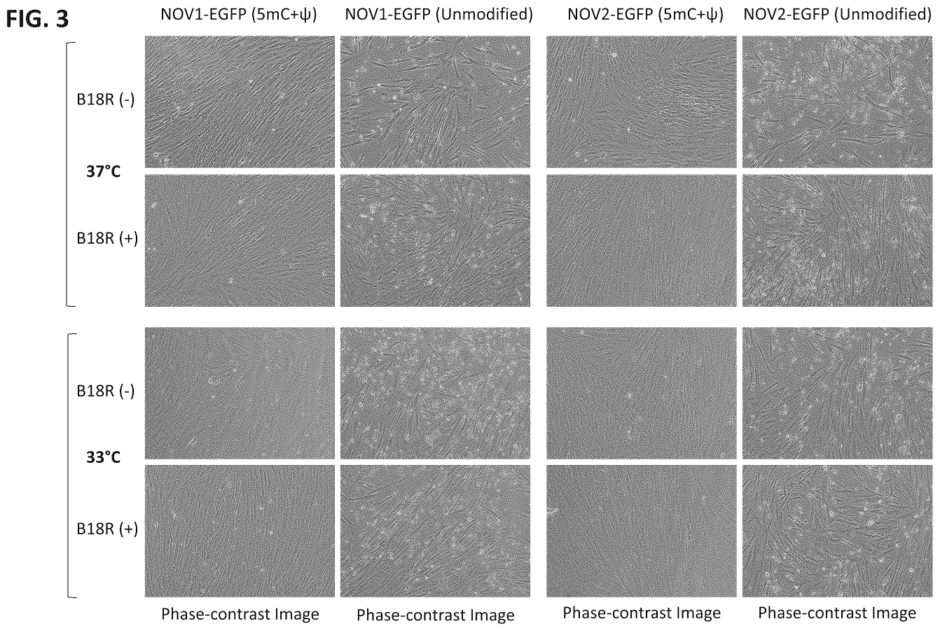

Cell viability and EGFP expression. Phase-contrast (left) and fluorescent (right) images were taken at 12 hours after RNA transfection (FIG. 2A, FIG. 2B). Fluorescent images showed the EGFP expression levels. Phase-contrast images were taken at 96 hours after RNA transfection (FIG. 3).

### Results and Conclusions

The expression of EGFP protein was detected by fluorescent microscopy in both NOV1-EGFP mRNA-transfected cells and NOV2-EGFP mRNA-transfected cells at 37° C. (standard cell culture condition) (FIG. 2A) and at 33° C. (reduced temperature condition) (FIG. 2B). Both mRNAs lack a poly(A) tail. As such, the synRNAs are translatable even though they lack a poly(A) sequence.

NOV is known to function optimally at a temperature of about 30° C., with little to no functionality at 37° C. Thus, stronger expression of EGFP at 37° C. (FIG. 2A) than at 33° C. (FIG. 2B) came as a surprise.

Interestingly, strong expression of EGFP was observed in more cells transfected with NOV2-EGFP mRNA than in cells transfected with NOV1-EGFP mRNA (FIG. 2A, FIG. 2B). Both the 5′-UTR and the 3′-UTR sequences of NOV2 are distinct from those of NOV1. This indicates that the efficiency of protein translation from mRNA lacking a poly(A) sequence is dependent on 5′-UTR and/or 3′-UTR sequences.

In the phase-contrast images, both NOV1-EGFP synRNA and NOV2-EGFP synRNA were observed to be toxic to the transfected cells (i.e., cytopathic effects), as demonstrated by the presence of dead cells (FIG. 2A, FIG. 2B, FIG. 3). Cytopathic effects were reduced in cells transfected with the nucleoside modified NOV1-EGFP synRNA and NOV2-EGFP synRNA (FIG. 2A, FIG. 2B, FIG. 3). Cytopathic effects of unmodified synRNAs were attenuated when transfected cells were cultured in the presence of B18R (FIG. 2A, FIG. 2B, FIG. 3). It is interesting to note that naturally-occurring NOV RNAs lack nucleoside modifications.

In summary, 5′-UTR and 3′-UTR of NOV1 and NOV2 can be used as components of poly(A)less synRNA for expression of a protein of interest in transfected cells. NOV2 (5′-UTR, 3′-UTR) works better than NOV1 (5′-UTR, 3′-UTR). Unmodified NOV1 and NOV2 mRNA are cytotoxic. Modification of nucleosides (5mC+Ψ) alleviates cytotoxicity of NOV1 and NOV2-based mRNAs. The presence of B18R in the culture medium alleviates cytotoxicity of mRNAs to some extent. NOV1 mRNA and NOV2 mRNA are translatable at both 37° C. and 33° C., with slightly higher levels of expression observed at 37° C. than 33° C.

### Example 3: Effects of Nucleoside Modifications on NOV2-EGFP Expression

This example describes the transfection of human fibroblast cells with nucleoside-modified NOV2-EGFP synRNA. Viability of transfected cells, as well as expression of EGFP protein from transfected cells is also described.

### Materials and Methods

Cell Culture. Human Dermal Fibroblasts, neonatal (HDFn) was purchased from ThermoFisher Scientific (Catalog No. C0045C) and cultured according to the manufacturer's instructions.

Transfection of NOV2-EGFP synRNA into HDFn. 3×10\hat{ }4 HDFn cells/well were plated in 24-well plate at day −1. The following day, cells were transfected with 0.5 μg of synRNA using MessengerMax transfection reagent (ThermoFisher). Five different synRNAs were transfected: NOV2-EGFP synRNA (Unmodified), NOV2-EGFP synRNA (modified with 5mC and Ψ), NOV2-EGFP synRNA (modified with m1Ψ), NOV2-EGFP synRNA (modified with 5moU), and NOV2-EGFP synRNA (modified with Ψ). HDFn cells were cultured at 37° C. or 33° C., with or without 250 ng/mL of B18R (Sigma).

Cell viability and EGFP expression. Phase-contrast and fluorescent images were taken 24 hours after RNA transfection. Fluorescent images showed the EGFP expression levels.

### Results and Conclusions

The expression of EGFP protein was detected in all cells transfected with modified NOV2-EGFP synRNAs when cultured at 37° C. (standard cell culture condition) and at 33° C. (reduced temperature condition). As shown in Table 3-1, synRNA containing N1-methylpseudouridine (m1Ψ) showed the strongest protein expression, followed by synRNA containing 5-methylcytosine and pseudouridine (5mC+Ψ). The modifications with 5moU or Ψshowed weaker expression than the modification with 5mC+Ψ.

TABLE 3-1
Effects of Culture Conditions and Nucleotide Modifications

| | GFP | 37° C. | 37° C. | 33° C. | 33° C. |
|---|---|---|---|---|---|
| synRNA | Intensity | B18R (+) | B18R (−) | B18R (+) | B18R (−) |
| NOV2-EGFP (Unmodified) | GFP > 30 | 40.2% | 35.7% | 34.9% | 37.0% |
| | GFP > 300 | 0.1% | 0.4% | 0.0% | 0.2% |
| NOV2-EGFP (5mC + Ψ) | GFP > 30 | 75.0% | 28.6% | 67.8% | 73.0% |
| | GFP > 300 | 6.6% | 3.7% | 5.1% | 5.6% |
| NOV2-EGFP (m1Ψ) | GFP > 30 | 83.0% | 82.9% | 74.0% | 74.0% |
| | GFP > 300 | 21.3% | 20.2% | 14.7% | 10.7% |
| NOV2-EGFP (5moU) | GFP > 30 | 50.8% | 65.5% | 58.3% | 68.1% |
| | GFP > 300 | 0.4% | 1.4% | 1.0% | 2.1% |
| NOV2-EGFP (Ψ) | GFP > 30 | 68.3% | 60.9% | 58.2% | 58.1% |
| | GFP > 300 | 2.9% | 1.9% | 0.6% | 0.6% |

Results of Example 3 were consistent with the results of Example 2. Modified NOV2-EGFP expression was slightly stronger when transfected cells were cultured at 37° C. than when cultured at 33° C. Unmodified NOV2 synRNA was more cytotoxic than modified NOV2 synRNAs, based on the phase-contrast images taken under the same conditions as in Table 3-1. B18R was able to alleviate cytotoxicity of unmodified NOV2 synRNA to some extent when transfected cells were cultured at 37° C.

In summary, 5′-UTR and 3′-UTR of NOV2 can be used as components of poly(A)less synRNA. NOV2 synRNA lacking nucleotide modifications are cytotoxic. Modification of nucleosides alleviates cytotoxicity of NOV2 mRNA. The presence of B18R in the culture medium of transfected cells, alleviates cytotoxicity of many NOV2 synRNAs to some extent, when the cells are cultured at 37° C. Protein production is somewhat greater when transfected cells are cultured at 37° C. than when cultured at 33° C.

### Example 4. Inclusion of a Multiple Cloning Site in NOV2-Based synRNAs

This example describes the testing of NOV2-based synRNAs in which the coding sequence of EGFP is inserted within a multiple cloning site located between the 5′-UTR and 3′-UTR of NOV2. This NOV2-based synRNA is named NOV2m (m for multiple cloning site). It can be used to produce mRNA containing any ORF (encoding any protein of interest), in the absence of a poly(A) sequence.

### Materials and Methods

Cell Culture. Human Dermal Fibroblasts, neonatal (HDFn) were purchased from ThermoFisher Scientific (Catalog No. C0045C) and cultured according to the manufacturer's instructions.

Transfection of NOV2-EGFP synRNA and NOV2m-EGFP synRNA into HDFn. 3×10\hat{ }4 HDFn cells/well were plated in 24-well plate at day −1. The following day, cells were transfected with 1.0 μg of NOV2-EGFP synRNA (modified with m1Ψ) or NOV2m-EGFP (modified with m1Ψ) synRNA with MessengerMax transfection reagent (ThermoFisher). HDFn cells were cultured at 37° C. or 30° C., with or without 250 ng/mL of B18R (Sigma).

Cell viability and EGFP expression. Phase-contrast and fluorescent images were taken at 17 hours, 24 hours, and 42 hours after RNA transfection. Fluorescent images showed the EGFP expression levels.

### Results and Conclusions

The expression of EGFP protein was detected in cells transfected with both NOV2-EGFP (m1Ψ) synRNA and NOV2m-EGFP (m1Ψ) synRNA when cultured at 37° C. in the presence or absence of B18R (Table 4-1). Strong expression of EGFP was observed for at least 42 hours after synRNA transfection. Although the presence of B18R increased the overall expression slightly, the m1Ψ-modification alone was sufficient for EGFP protein expression.

The expression of EGFP protein was detected in cells transfected with both NOV2-EGFP (m1Ψ) synRNA and NOV2m-EGFP (m1Ψ) synRNA when cultured at 30° C. in the presence or absence of B18R (Table 4-1). Although the strong expression was observed for at least 42 hours after the synRNA transfection, the expression was stronger at 37° C. than 30° C. Although the presence of B18R increased the overall expression slightly, the m1Ψ-modification alone was sufficient to express the protein strongly.

TABLE 4-1
Effects of Culture Conditions and Multiple Cloning Site

| | GFP Intensity | NOV2-EGFP (m1Ψ) | NOV2m-EGFP (m1Ψ) |
|---|---|---|---|
| 37° C., B18R (+) | | | |
| 24 hrs | GFP > 30 | 91.6% | 91.5% |
| | GFP > 300 | 62.2% | 61.7% |
| 46 hrs | GFP > 30 | 89.6% | 90.3% |
| | GFP > 300 | 52.5% | 54.3% |
| 37° C., B18R (−) | | | |
| 24 hrs | GFP > 30 | 85.6% | 87.6% |
| | GFP > 300 | 48.4% | 51.2% |
| 46 hrs | GFP > 30 | 87.6% | 86.0% |
| | GFP > 300 | 43.1% | 40.8% |
| 30° C., B18R (+) | | | |
| 24 hrs | GFP > 30 | 84.6% | 86.5% |
| | GFP > 300 | 36.1% | 44.4% |
| 46 hrs | GFP > 30 | 85.0% | 85.3% |
| | GFP > 300 | 34.5% | 39.6% |
| 30° C., B18R (−) | | | |
| 24 hrs | GFP > 30 | 78.6% | 82.5% |
| | GFP > 300 | 30.9% | 35.2% |
| 46 hrs | GFP > 30 | 77.4% | 77.9% |
| | GFP > 300 | 28.2% | 29.1% |

In summary, insertion of a multiple cloning site between 5′-UTR and 3′-UTR of NOV2 did not interfere with translation of protein from NOV2-based synRNA. A plasmid including the NOV2m sequence facilitates the cloning of a coding sequence of interest into the multiple cloning site for in vitro transcription of synRNA lacking a poly(A) tail. Inclusion of modified nucleosides alleviates cytotoxicity of NOV2m synRNAs, based on the phase-contrast images taken under the same conditions as in Table 3-1. The presence of B18R in the culture medium of transfected cells increases protein expression to a modest extent from synRNAs containing modified nucleotides. Protein production is somewhat stronger when transfected cells were cultured at 37° C. than when cultured at 30° C. Protein production from NOV2m synRNA continues for more than 42 hours after transfection.

### Example 5. Stronger and Longer Protein Expression from NOV2-RNA and NOV2m-RNA

This example describes the comparison of EGFP expression from cells transfected with NOV2 and NOV2m synRNA versus to cells transfected with a Control synRNA with the standard 120 poly(A) tail.

### Materials and Methods

Production of a Control-EGFP synRNA by in vitro transcription. As a control, a Control-EGFP synRNA encoding EGFP was used. The Control-EGFP synRNA was made according to a published protocol with 5-methylcytosine and pseudouridine (5mC+Ψ) modifications (Warren et al., 2010; Mandall and Rossi, 2013). A poly(A) tail of 120 consecutive adenines was added to the 3′ end by a tail-PCR (Warren et al., 2010; Mandall and Rossi, 2013). The 3′-UTR sequence of Control-EGFP synRNA is identical to the Mus musculus hemoglobin alpha, adult chain 1 (Hba-al), mRNA (NM_008218.2).

Cell Culture. Human Dermal Fibroblasts, neonatal (HDFn) were purchased from ThermoFisher Scientific (Catalog No. C0045C) and cultured according to the manufacturer's instructions.

Transfection of NOV2-EGFP synRNA, NOV2m-EGFP synRNA, and synRNA-EGFP into HDFn. 3×10\hat{ }4 HDFn cells/well were plated in 24-well plate at day −1. The following day, cells were transfected with 1.0 μg of NOV2-EGFP synRNA (modified with m1Ψ), NOV2m-EGFP synRNA (modified with m1Ψ), or synRNA-EGFP (modified with 5mC+Ψ) using MessengerMax transfection reagent (ThermoFisher). HDFn cells were cultured at 37° C., with or without 250 ng/mL of B18R (Sigma).

Cell viability and EGFP expression. EGFP expression levels (fluorescence intensities) were measured by Moxi GO II (ORFLO) at 24 hours, 96 hours, and 185 hours after transfection. GFP-positive (+) cells were presented as a percent (%) of total cells at the fluorescence intensity of GFP>30 for all GFP+ cells or GFP>300 for strong GFP+ cells.

### Results and Conclusions

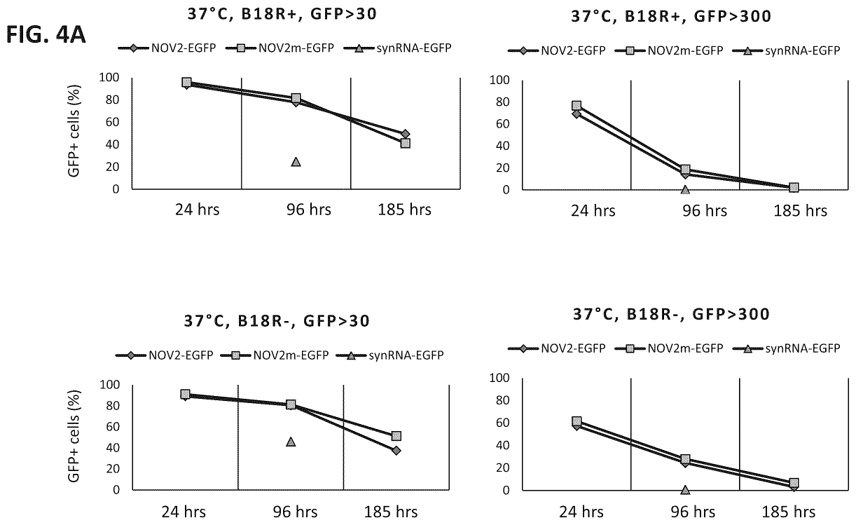

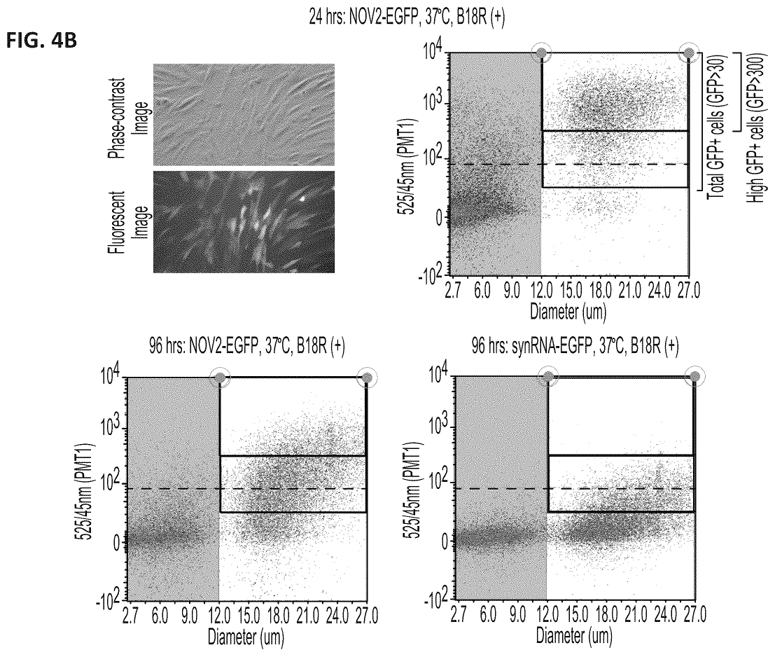

Expression of EGFP protein was detected in cells transfected with both NOV2-EGFP (m1Ψ) synRNA and NOV2m-EGFP (m1Ψ) synRNA when transfected cells were cultured at 37° C. or 30° C., in the presence or absence of B18R (FIG. 4A, FIG. 4B, Table 5-1). At 24 hours post-transfection, nearly all cells expressed GFP in the presence and absence of B18R (GFP>30). Around 60% to 80% of the GFP+ cells showed high levels of GFP expression (GFP>300). For both NOV2-EGFP synRNA and NOV2m-EGFP synRNA, GFP expression continued beyond 185 hours (8 days) (FIG. 4A, FIG. 4B, Table 5-1).

As expected, the GFP was also expressed by cells transfected with a Control-EGFP synRNA, which included a 120A poly(A) tail (FIG. 4A, Table 5-1). However, expression from poly(A)-tailed synRNA was much weaker than from NOV2 and NOV2m synRNA, which are devoid of poly(A) tails (FIG. 4A, Table 5-1; at 96 hours post-transfection).

In summary, protein production from NOV2 and NOV2m synRNA continues for more than 185 hours post-transfection. Poly(A)less NOV2 and NOV2m synRNA show comparable to or even greater protein expression than the Control-EGFP synRNA with 120 A poly(A).

TABLE 5-1
Effects of Culture Conditions and Poly(A) Tail

| Conditions | RNA | GFP Intensity | 24 hr | 96 hr | 185 hr |
|---|---|---|---|---|---|
| 30° C., B18R (+) | NOV2-EGFP | GFP > 30 | 89.9% | 93.0% | 83.6% |
| | (0A) | GFP > 300 | 55.4% | 54.4% | 20.7% |
| | NOV2m-EGFP | GFP > 30 | 92.1% | 93.4% | 78.8% |
| | (0A) | GFP > 300 | 61.5% | 61.2% | 22.5% |
| | Control-EGFP | GFP > 30 | | 93.0% | |
| | synRNA (120A) | GFP > 300 | | 62.7% | |
| 30° C., B18R (−) | NOV2-EGFP | GFP > 30 | 92.6% | 85.5% | 71.1% |
| | (0A) | GFP > 300 | 58.3% | 42.2% | 16.8% |
| | NOV2m-EGFP | GFP > 30 | 92.9% | 86.4% | 79.2% |
| | (0A) | GFP > 300 | 60.6% | 43.9% | 24.2% |
| | Control-EGFP | GFP > 30 | | 74.4% | |
| | synRNA (120A) | GFP > 300 | | 18.8% | |
| 37° C., B18R (+) | NOV2-EGFP | GFP > 30 | 93.6% | 77.8% | 49.5% |
| | (0A) | GFP > 300 | 69.2% | 14.1% | 2.0% |
| | NOV2m-EGFP | GFP > 30 | 95.8% | 81.6% | 41.2% |
| | (0A) | GFP > 300 | 76.7% | 18.6% | 2.2% |
| | Control-EGFP | GFP > 30 | | 24.8% | |
| | synRNA (120A) | GFP > 300 | | 0.3% | |
| 37° C., B18R (−) | NOV2-EGFP | GFP > 30 | 89.0% | 80.5% | 37.4% |
| | (0A) | GFP > 300 | 57.3% | 24.4% | 3.0% |
| | NOV2m-EGFP | GFP > 30 | 90.9% | 81.1% | 51.2% |
| | (0A) | GFP > 300 | 61.6% | 27.9% | 6.7% |
| | Control-EGFP | GFP > 30 | | 46.0% | |
| | synRNA (120A) | GFP > 300 | | 0.7% | |

### Example 6. Production of DENVm-EGFP SynRNAs and Capless-DENVm-EGFP Synrnas

This example describes the construction of plasmid DNAs and their use for production of synRNAs. Dengue virus (DENV) is a non-segmented +ssRNA virus with 11 kb single RNA genome, which is modified at the 5′ end with a cap-1 structure for canonical cellular translation. The 3Yend of the RNA genome does not carry poly(A) tail, rather it forms a loop structure. Interestingly, cap-independent mechanisms of translation have also been described for DENV (Mazeaud et al., 2018). Therefore, we tested both Cap1 and Cap-less versions of DENV RNAs.

### Materials and Methods

Design of DENVm synRNAs and construction of template plasmid DNAs. Genome sequence of Dengue virus 2 (DENV) (NC_001474.2; Kinney et al., 1997) was used as starting sequences. Although DENV RNA genomes appear simple, consisting of 5′-UTR, a single CDS, and 3′-UTR, we anticipated that a simple strategy of replacing the CDS with multiple cloning site sequence for cloning a foreign CDS would not work. For DENV RNA genomes to function properly, it is known that DENV RNA must be circularized by the complementary sequences located at both 5′-UTR (including sequences downstream of an ATG initiation codon) and 3′-UTR (Mazeaud et al., 2018). Therefore, we assumed that an N-terminal part of CDS have to be kept in synRNA structure. To make a DENV-based synRNA construct that allows the insertion of a foreign CDS starting from an ATG to a stop codon, we introduced two mutations that change AUG to AUC in two locations (FIG. 5B). These mutations eliminate two ATG start codons upstream of a multiple cloning site. However, the introduction of these two mutations caused the disruption of complementary sequences between these sequences and corresponding sequences in the 3′-UTR. To maintain the circularization structure of a DENV-based synRNA, two corresponding mutations in 3′-UTR (CAU->GAU; GAC->CAC) were also introduced (FIG. 5B).

The final DENV-based synRNA construct (named DENVm synRNA) contains 5′-Cap1, 5′-UTR (mutated), multiple cloning site (MCS), 3′-UTR (mutated), and no poly(A) (SEQ ID NO:11). The RNAs were made by IVT using T7 RNA polymerase and plasmid DNA templates linearized with a SapI restriction enzyme.

An exemplary plasmid DNA for production of DENVm synRNA contains the T7 RNA polymerase promoter sequence for in vitro transcription (IVT) reaction. A 5′-Cap can be added to mRNA using standard methods. However, for convenience, CleanCap AG (Henderson 2021; TriLink) was used to add a 5′-Cap (Cap1). This was made possible by insertion of nucleotide (A) immediately downstream of the T7 promoter. Any 5′-Cap (e.g., Cap0, Cap1, Cap2) can be added to the 5′-end of DENVm synRNA. At the 3′end, a SapI restriction enzyme site was added to produce the same 3′end sequence as is present in the DENV RNA genome. To test the protein production, EGFP was cloned into the MCS.

Design of Capless-DENVm synRNAs and construction of template plasmid DNAs. Because cap-independent mechanisms of translation have also been described for DENV (Mazeaud et al., 2018), we constructed a plasmid DNA that has a standard T7 RNA polymerase promoter, which produces the same DENVm synRNA, but lacks the 5′-Cap, named Capless-DENVm synRNA (SEQ ID NO:12).

Production of DENVm-EGFP synRNA by in vitro transcription. The plasmid DNAs were linearized with the SapI restriction enzyme and used as a template DNA for in vitro transcription (IVT), which was carried using MEGAscript T7 Kit (ThermoFisher Scientific), according to the manufacturer's instructions. A 5′-Cap was incorporated using CleanCap AG (TriLink). Two versions of RNAs were prepared: a standard RNA without any nucleoside modifications (Unmodified or UNM) and RNA modified with N1-methylpseudouridine (m1Ψ).

### Results and Conclusions

Schematic diagrams of DENVm-EGFP synRNAs that were successfully produced are shown in FIG. 5A, FIG. 5B, and FIG. 5C. The RNA sequence of DENVm-MCS is set forth as SEQ ID NO:11, and the RNA sequence of Capless-DENVm is set forth as SEQ ID NO:12.

### Example 7. EGFP Protein Expression from DENVm synRNA and Cap1Ess-DENVm synRNA

This example describes the comparison of EGFP expression from cells transfected with DENVm-EGFP synRNA and Capless-DENVm-EGFP synRNA.

### Materials and Methods

Cell Culture. Human Dermal Fibroblasts, neonatal (HDFn) were purchased from ThermoFisher Scientific (Catalog No. C0045C) and cultured according to the manufacturer's instructions.

Transfection of DENVm-EGFP synRNA, and Capless-DENVm-EGFP synRNA into HDFn. 3×10\hat{ }4 HDFn cells/well were plated in 24-well plate at day −1. The following day, cells were transfected with 1.0 μg of synRNAs using MessengerMax transfection reagent (ThermoFisher). HDFn cells were cultured at 30° C. or 37° C., with or without 250 ng/mL of B18R (Sigma).

EGFP expression. EGFP expression levels (fluorescence intensities) were measured by Moxi GO II (ORFLO) on Day 1 and Day 4 after transfection. GFP-positive (+) cells were presented as a percent (%) of total cells at the fluorescence intensity of GFP>30 for all GFP+ cells, GFP>300 for strong GFP+ cells, or GFP>2000 for very strong GFP+ cells.

### Results and Conclusions

Expression of EGFP protein was detected in cells transfected with both DENVm-EGFP synRNA (unmodified) and DENVm-EGFP synRNA (m1Ψ) when transfected cells were cultured at 37° C. or 30° C., in the presence or absence of B18R (FIG. 6).

Interestingly, in all four culture conditions (30° C. or 37° C.; B18R+ or B18R−), DENVm-EGFP synRNA (unmodified) showed stronger EGFP expression than DENVm-EGFP synRNA (m1Ψ) (FIG. 6). This is a very unique feature of DENVm synRNA, as it is well known that in general synRNA with modified nucleosides show stronger protein expression than unmodified synRNA.

It is also unusual that DENVm-EGFP synRNA (unmodified) showed stronger expression on Day 4 compared to Day 1 (FIG. 6), as synRNAs in general show strong expression on Day 1, which gradually gets weaker over time.

It is also interesting to note that the EGFP expression was not influenced much by the presence or absence of B18R (FIG. 6).

In contrast to 5′-Cap+ version, Capless version of both DENVm-EGFP synRNA (unmodified) and DENVm-EGFP synRNA (m1Ψ) produced very low levels of EGFP (FIG. 7). This result suggests that 5′-Cap is required for efficient translation from DENVm RNA, as is the case for their natural state, although cap-independent mechanisms of translation have been described for DENV earlier (Mazeaud et al., 2018).

In summary, DENVm synRNA provides unique features distinct from commonly used synRNA platforms: notably, protein production from DENVm synRNA is stronger in natural unmodified form than in modified nucleoside form.

### Example 8. EGFP Protein Expression from DENVm synRNA for Long-Term Cell Culture

This example describes the long-term EGFP expression from DENVm-EGFP synRNA.

### Materials and Methods

Cell Culture. Human Dermal Fibroblasts, neonatal (HDFn) were purchased from ThermoFisher Scientific (Catalog No. C0045C) and cultured according to the manufacturer's instructions.

Transfection of DENVm-EGFP synRNA (unmodified), and DENVm-EGFP synRNA (modified with m1Ψ) into HDFn. 3×10\hat{ }4 HDFn cells/well were plated in 24-well plate at day −1. The following day, cells were transfected with 1.0 μg of synRNAs using MessengerMax transfection reagent (ThermoFisher). HDFn cells were cultured at 33° C. or 37° C., with or without 250 ng/mL of B18R (Sigma) for up to 11 days after synRNA transfection.

EGFP expression. EGFP expression levels (fluorescence intensities) were measured by Moxi GO II (ORFLO) at various time points after transfection. GFP-positive (+) cells were presented as a percent (%) of total cells at the fluorescence intensity of GFP>30 for all GFP+ cells, GFP>300 for strong GFP+ cells, or GFP>2000 for very strong GFP+ cells.

### Results and Conclusions

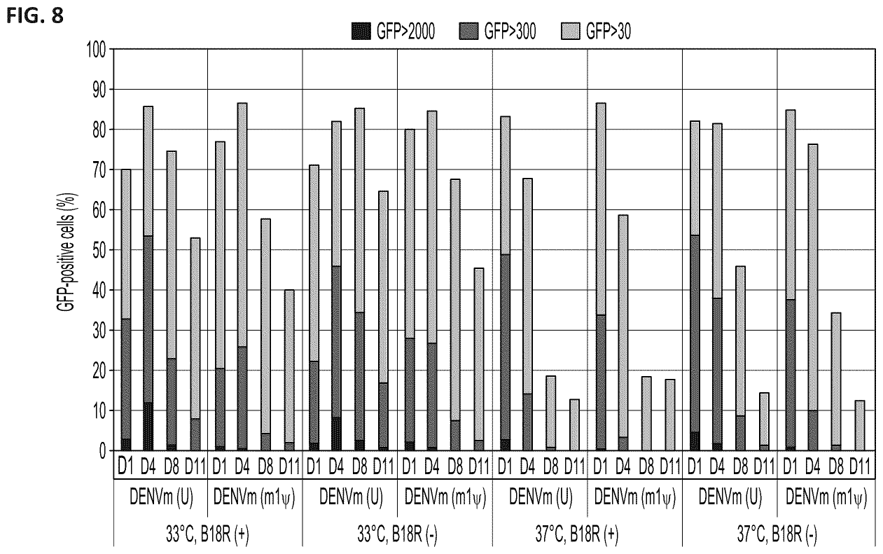

Consistent with the EXAMPLE 7, interestingly, in all four culture conditions (33° C. or 37° C.; B18R+ or B18R−), DENVm-EGFP synRNA (unmodified) showed stronger EGFP expression than DENVm-EGFP synRNA (m1Ψ) (FIG. 8). EGFP expression was not influenced much by the presence or absence of B18R (FIG. 8). In the 33° C. culture condition, the EGFP expression increased from Day 1 to Day 4, but subsequently decreased over time and became very low by Dy 11. However, in the 37° C. culture condition, the EGFP expression started high on Day 1, and gradually decreased over time (FIG. 8).

In summary, the experiments show that DENVm functions as a unique synRNA platform. It may be particularly useful for applications that require nucleoside-unmodified synRNAs.

### Example 9. Production of Plant +ssRNA Virus-Based synRNAs without Cap

This example describes the construction of plasmid DNAs and their use for production of synRNAs based on +ssRNA viruses that infect plants. Unlike +ssRNA viruses that infect insects and vertebrates, +ssRNA viruses that infect plants do not have either 5′-Cap structure nor poly(A) tails. We selected 5 different plant +ssRNA viruses and made synRNA constructs. These plant +ssRNAs do not have 5′-Cap and thus we first tested Capless-version of synRNAs.

### Materials and Methods

Design of Plant +ssRNA Virus-Based synRNAs and Construction of Template Plasmid DNAs.

Capless-BYDVm synRNA (SEQ ID NO:14) consists of 5′-UTR, multiple cloning sites (MCS), and 3′-UTR (without BTE) of Barley yellow dwarf virus (BYDV: NC_004750.1). The BTE sequence element is the 3′CITE which folds into a compact cruciform RNA secondary structure and is termed the BYDV-like translation element (BTE).

Capless-BYDV2m RNA (SEQ ID NO:16) contains 5′-UTR, MCS, BTE (added) sequence, and 3′-UTR of Barley yellow dwarf virus (BYDV: NC_004750.1).

Capless-MNESVm RNA (SEQ ID NO:18) contains 5′-UTR, MCS, and 3′-UTR of Maize necrotic streak virus (MNESV: NC_007729.1).

Capless-PMVm RNA (SEQ ID NO:20) contains 5′-UTR, MCS, and 3′-UTR of Panicum mosaic virus (PMV: U55002.1).

Capless-PEMV2m RNA (SEQ ID NO:22) contains 5′-UTR, MCS, and 3′-UTR of Pea enation mosaic virus-2 (PEMV2: NC_003853.1).

Capless-TCVm RNA (SEQ ID NO:24) contains 5′-UTR, MCS, and 3′-UTR of Turnip crinkle virus RNA for coat protein (TCV: NC_003821.3).

An exemplary plasmid DNA for production of plant +ssRNA virus-based synRNA contains the T7 RNA polymerase promoter sequence for in vitro transcription (IVT) reaction.

Production of synRNA by in vitro transcription. The plasmid DNAs were linearized with the SapI restriction enzyme and used as a template DNA for in vitro transcription (IVT), which was carried using MEGAscript T7 Kit (ThermoFisher Scientific), according to the manufacturer's instructions. Two versions of RNAs were prepared: a standard RNA without any nucleoside modifications (Unmodified or UNM or U) and RNA modified with N1-methylpseudouridine (m1Ψ or M).

### Results and Conclusions

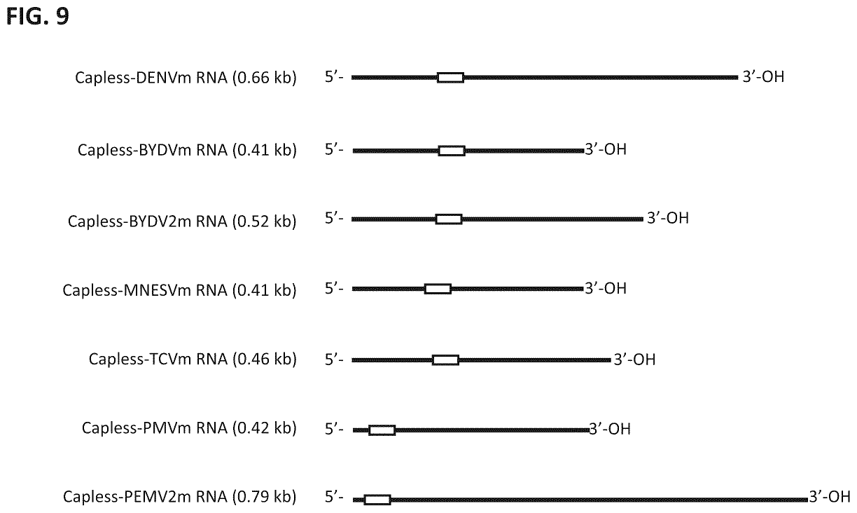

Schematic diagrams of synRNAs that were successfully produced are shown in FIG. 9. The RNA sequences of these constructs are set forth as SEQ ID NOS: 14, 16, 18, 20, 22, 24.

### Example 10. EGFP Protein Expression from Plant +ssRNA Virus-Based synRNAs without Cap (Cap1Ess)

This example describes the EGFP expression from plant +ssRNA virus-based synRNAs encoding for EGFP.

### Materials and Methods

Cell Culture. Human Dermal Fibroblasts, neonatal (HDFn) were purchased from ThermoFisher Scientific (Catalog No. C0045C) and cultured according to the manufacturer's instructions.

Transfection of Capless-BYDm, Capless-MNESVm, Capless-PMVm, Capless-TCVm, Capless-PEMV2m, and Capless-BYDV2m synRNAs encoding for EGFP unmodified and modified with m1Ψ into HDFn. 3×10\hat{ }4 HDFn cells/well were plated in 24-well plate at day −1. The following day, cells were transfected with 1.0 μg of synRNAs using MessengerMax transfection reagent (ThermoFisher). HDFn cells were cultured at 33° C. or 37° C., with or without 250 ng/mL of B18R (Sigma) for up to 5 days after synRNA transfection.

EGFP expression. EGFP expression levels (fluorescence intensities) were measured by Moxi GO II (ORFLO) at various time points after transfection. GFP-positive (+) cells were presented as a percent (%) of total cells at the fluorescence intensity of GFP>30 for all GFP+ cells, GFP>300 for strong GFP+ cells, or GFP>2000 for very strong GFP+ cells.

### Results and Conclusions

Although some synRNAs showed a very low EGFP expression (e.g., Capless-PMVm, Capless-BYDVm, and Capless-BYDV2m), essentially no EGFP expression was detected in all four culture conditions (30° C. or 37° C.; B18R+ or B18R−), irrespective of unmodified or modified nucleosides (Table 10-1).

TABLE 10-1
EGFP expression from plant +ssRNA virus-based synRNAs without cap (capless)

| | | Day 1 | | | | Day 2 | | | |
|---|---|---|---|---|---|---|---|---|---|
| | | 30° C. | 30° C. | 37° C. | 37° C. | 30° C. | 30° C. | 37° C. | 37° C. |
| | | B18R+ | B18R− | B18R+ | B18R− | B18R+ | B18R− | B18R+ | B18R− |
| Capless-BYDVm | U | (−) | (−) | (−) | (−) | (−) | (−) | (−) | (−) |
| Capless-BYDVm | M | (−) | (−) | (−) | (−) | (−) | (−) | | |
| Capless-MNESVm | U | (−) | (−) | (−) | (−) | (−) | (−) | (−) | (−) |
| Capless-MNESVm | M | (−) | (−) | (−) | (−) | (−) | (−) | | |
| Capless-PMVm | U | (±) | (−) | (−) | (−) | (±) | (±) | | |
| Capless-PMVm | M | (−) | (−) | (−) | (−) | (−) | (−) | | |
| Capless-TCVm | U | (−) | (−) | (−) | (−) | (−) | (−) | | |
| Capless-TCVm | M | (−) | (−) | (−) | (−) | (−) | (−) | | |
| Capless-PEMV2m | U | (−) | (−) | (−) | (−) | (−) | (−) | (−) | (−) |
| Capless-PEMV2m | M | (−) | (−) | (−) | (−) | (−) | (−) | (−) | (−) |
| Capless-BYDV2m | U | 0.7 | 1.1 | 1.8 | 2.0 | (−) | (−) | (±) | (±) |
| Capless-BYDV2m | M | 4.8 | 6.1 | 0 | 2.6 | (±) | (±) | (±) | (±) |
| | | Day 3 | | | | Day 4 | | | |
| | | 30° C. | 30° C. | 37° C. | 37° C. | 30° C. | 30° C. | 37° C. | 37° C. |
| | | B18R+ | B18R− | B18R+ | B18R− | B18R+ | B18R− | B18R+ | B18R− |
| Capless-BYDVm | U | (−) | (−) | (−) | (−) | 0.9 | 1.3 | 2.8 | 14 |
| Capless-BYDVm | M | | | | | (−) | (−) | (−) | (−) |
| Capless-MNESVm | U | (−) | (−) | (−) | (−) | 1.1 | 1.5 | 2.4 | 12 |
| Capless-MNESVm | M | | | | | (±) | (−) | (−) | (−) |
| Capless-PMVm | U | | | | | (±) | (±) | (−) | (−) |
| Capless-PMVm | M | | | | | (−) | (−) | (−) | (−) |
| Capless-TCVm | U | | | | | (−) | (−) | (−) | (−) |
| Capless-TCVm | M | | | | | (−) | (±) | (−) | (−) |
| Capless-PEMV2m | U | 0.7 | 0.7 | 0.5 | 5.2 | (−) | (−) | (−) | (±) |
| Capless-PEMV2m | M | 0.9 | 2.2 | 0.4 | 0.8 | (−) | (−) | (±) | (±) |
| Capless-BYDV2m | U | (±) | (±) | (±) | (±) | | | | |
| Capless-BYDV2m | M | (±) | (±) | (±) | (±) | | | | |

Day 5

| | | 30° C. | 30° C. | 37° C. | 37° C. |
|---|---|---|---|---|---|
| | | B18R+ | B18R− | B18R+ | B18R− |
| Capless-BYDVm | U | | | | |
| Capless-BYDVm | M | (−) | (−) | (−) | (−) |
| Capless-MNESVm | U | | | | |
| Capless-MNESVm | M | (−) | (−) | (−) | (−) |
| Capless-PMVm | U | 42 | | 2.4 | |
| Capless-PMVm | M | 1.6 | | 1 | |
| Capless-TCVm | U | (−) | (−) | (−) | (−) |
| Capless-TCVm | M | (−) | (−) | (−) | (−) |
| Capless-PEMV2m | U | (−) | (−) | (±) | (±) |
| Capless-PEMV2m | M | (−) | (−) | (±) | (±) |
| Capless-BYDV2m | U | 12 | 15 | 4.5 | 29 |
| Capless-BYDV2m | M | 5.9 | 18 | 2.8 | 25 |

In summary, the experiments show that Capless version of plant +ssRNA virus-based synRNAs do not express the EGFP.

### Example 11. Production of Plant +ssRNA Virus-Based synRNAs with 5′-Cap

This example describes the construction of plasmid DNAs and their use for production of synRNAs based on +ssRNA viruses that infect plants. Unlike +ssRNA viruses that infect insects and vertebrates, +ssRNA viruses that infect plants do not have either 5′-Cap structure nor poly(A) tails. We artificially added 5′-Cap (Cap1) to the 5′-end of synRNAs. We selected 5 different plant +ssRNA viruses and made synRNA constructs.

### Materials and Methods

Design of plant +ssRNA virus-based synRNAs with 5′-Cap and construction of template plasmid DNAs.

BYDVm synRNA (SEQ ID NO:13) consists of 5′-Cap (Cap1), 5′-UTR, multiple cloning sites (MCS), and 3′-UTR (without BTE) of Barley yellow dwarf virus (BYDV: NC_004750.1). The BTE sequence element is the 3′CITE which folds into a compact cruciform RNA secondary structure and termed the BYDV-like translation element (BTE).

BYDV2m RNA (SEQ ID NO:15) contains 5′-Cap (Cap1), 5′-UTR, MCS, BTE sequence (added), and 3′-UTR of Barley yellow dwarf virus (BYDV: NC_004750.1).

MNESVm RNA (SEQ ID NO:17) contains 5′-Cap (Cap1), 5′-UTR, MCS, and 3′-UTR of Maize necrotic streak virus (MNESV: NC_007729.1).

PMVm RNA (SEQ ID NO:19) contains 5′-Cap (Cap1), 5′-UTR, MCS, and 3′-UTR of Panicum mosaic virus (PMV: U55002.1).

PEMV2m RNA (SEQ ID NO:21) contains 5′-Cap (Cap1), 5′-UTR, MCS, and 3′-UTR of Pea enation mosaic virus-2 (PEMV2: NC_003853.1).

TCVm RNA (SEQ ID NO:23) contains 5′-Cap (Cap1), 5′-UTR, MCS, and 3′-UTR of Turnip crinkle virus RNA for coat protein (TCV: NC_003821.3).

An exemplary plasmid DNA for production of plant +ssRNA virus-based synRNA contains the T7 RNA polymerase promoter sequence for in vitro transcription (IVT) reaction. A 5′-Cap can be added to mRNA using standard methods. However, for convenience, CleanCap AG (Henderson 2021; TriLink) was used to add a 5′-Cap (Cap1). This was made possible by insertion of nucleotide (A) immediately downstream of the T7 promoter. Any 5′-Cap (e.g., Cap0, Cap1, Cap2) can be added to the 5′-end of synRNA. At the 3′end, a SapI restriction enzyme site was added to produce the same 3′end sequence as is present in the +ssRNA virus RNA genome. To test the protein production, EGFP was cloned into the MCS.

Production of synRNA by in vitro transcription. The plasmid DNAs were linearized with the SapI restriction enzyme and used as a template DNA for in vitro transcription (IVT), which was carried using MEGAscript T7 Kit (ThermoFisher Scientific), according to the manufacturer's instructions. Two versions of RNAs were prepared: a standard RNA without any nucleoside modifications (Unmodified or UNM or U) and RNA modified with N1-methylpseudouridine (m1Ψ or M).

### Results and Conclusions

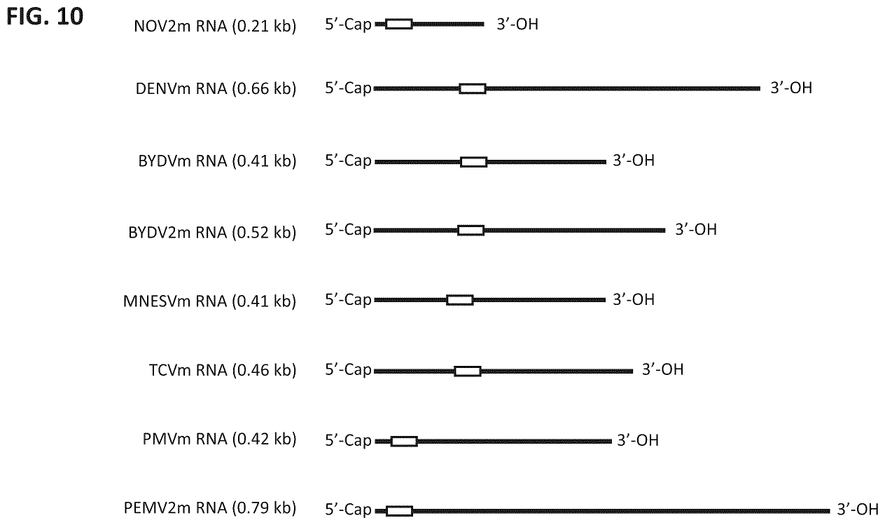

Schematic diagrams of synRNAs that were successfully produced are shown in FIG. 10. The RNA sequences of these constructs are set forth as SEQ ID NOS:13, 15, 17, 19, 21, 23.

### Example 12. EGFP Expression from Plant +ssRNA Virus-Based synRNAs with 5′-Cap

This example describes the EGFP expression from plant +ssRNA virus-based synRNAs (with 5′-Cap) encoding for EGFP.

### Materials and Methods

Cell Culture. Human Dermal Fibroblasts, neonatal (HDFn) were purchased from ThermoFisher Scientific (Catalog No. C0045C) and cultured according to the manufacturer's instructions.

Transfection of BYDVm, MNESVm, PMVm, TCVm, PEMV2m, and BYDV2m synRNAs encoding for EGFP unmodified and modified with m1Ψ into HDFn. 3×10\hat{ }4 HDFn cells/well were plated in 24-well plate at day −1. The following day, cells were transfected with 1.0 μg of synRNAs using MessengerMax transfection reagent (ThermoFisher). HDFn cells were cultured at 33° C. or 37° C., with or without 250 ng/mL of B18R (Sigma) for up to 4 days after synRNA transfection.

Transfection of DENVm-EGFP synRNA (unmodified), DENVm-EGFP synRNA (modified with m1Ψ), NOV2-EGFP synRNA (modified with m1Ψ), and NOV2m-EGFP synRNA (modified with m1Ψ) into HDFn. These four synRNAs were used for comparison purposes.

Transfection of poly(A)less Control-EGFP synRNA (modified with m1Ψ) into HDFn. For comparison purposes, this Control synRNA-EGFP was made by a published protocol (Warren et al., 2010; Mandall and Rossi, 2013). The 3′-UTR sequence of synRNA-EGFP is identical to the Mus musculus hemoglobin alpha, adult chain 1 (Hba-a1), mRNA (NM_008218.2). The RNA was made with the nucleoside modification with m1Ψ. Instead of adding poly(A) tail of 120 consecutive adenine nucleotides to the 3′ end, no poly(A) was added to this poly(A)less synRNA-EGFP by a tail-PCR.

EGFP expression. EGFP expression levels (fluorescence intensities) were measured by Moxi GO II (ORFLO) on Day 1 and Day 4 various after transfection. GFP-positive (+) cells were presented as a percent (%) of total cells at the fluorescence intensity of GFP>30 for all GFP+ cells, GFP>300 for strong GFP+ cells, or GFP>2000 for very strong GFP+ cells.

### Results and Conclusions

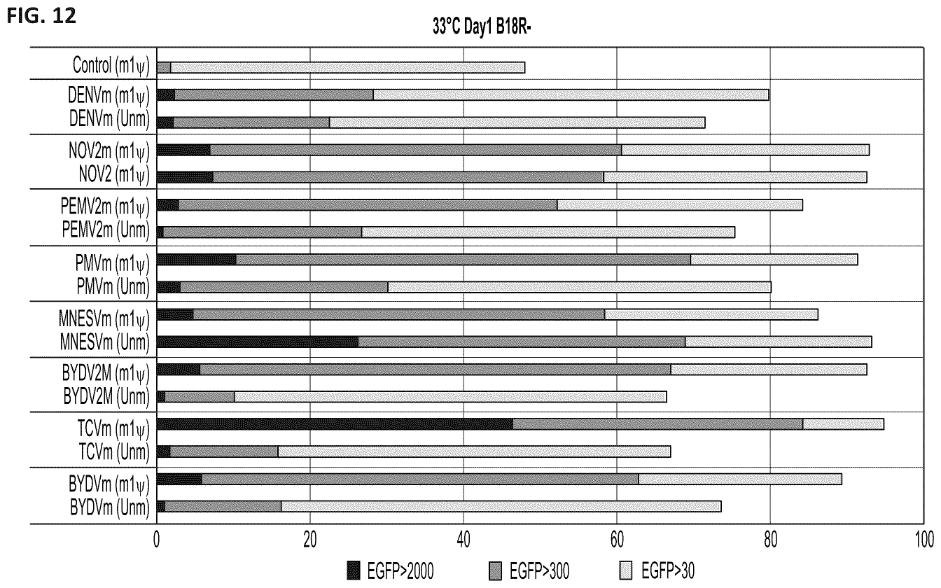

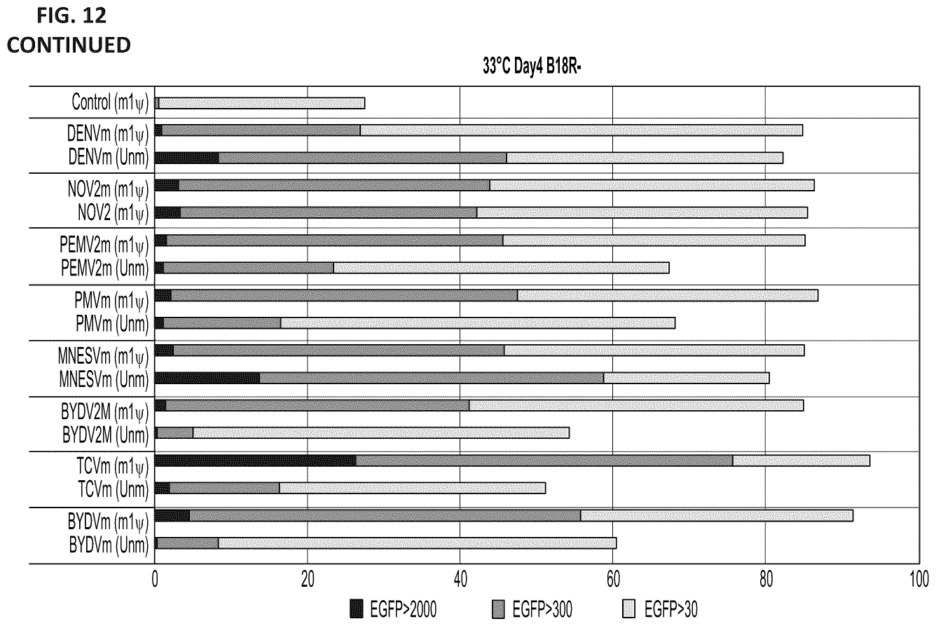

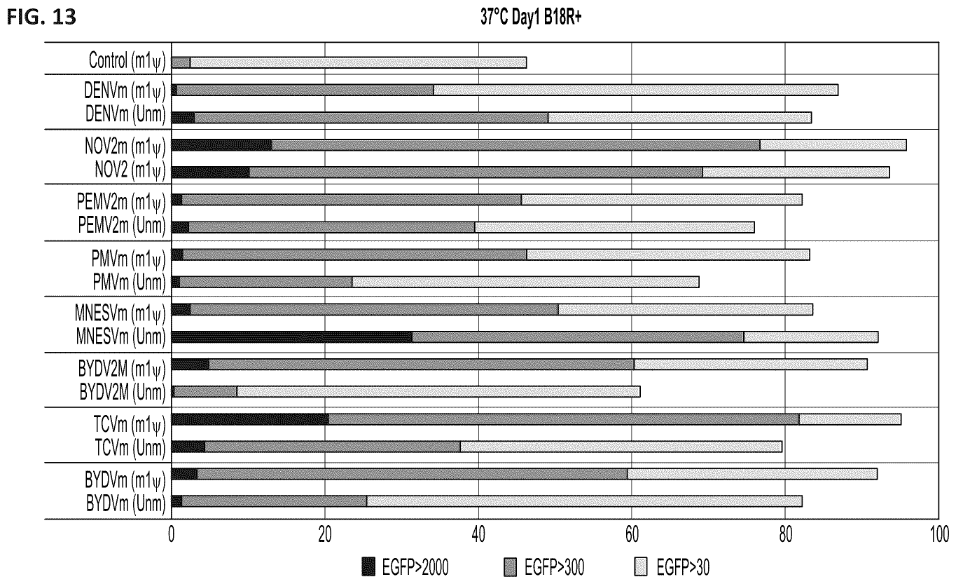

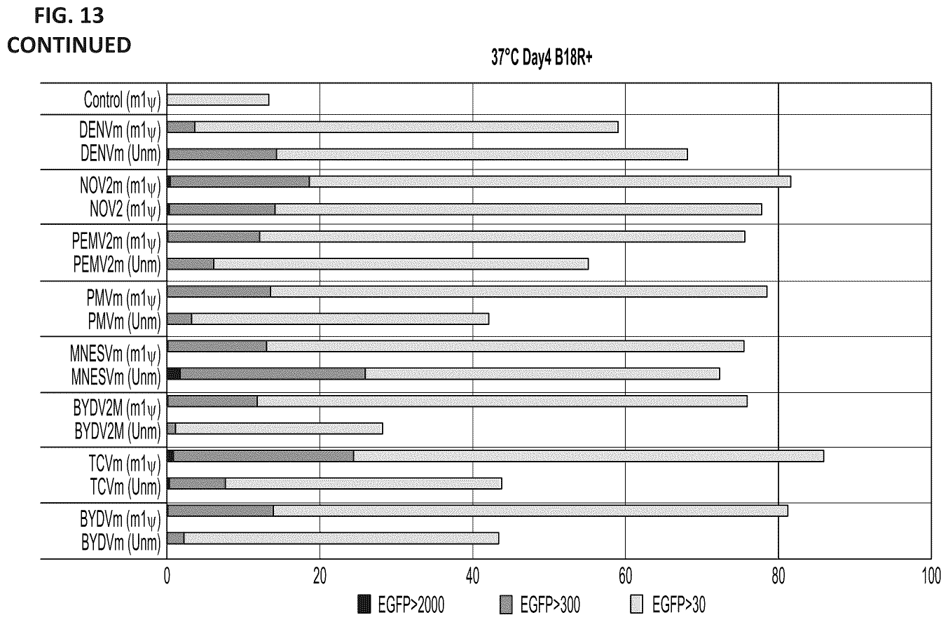

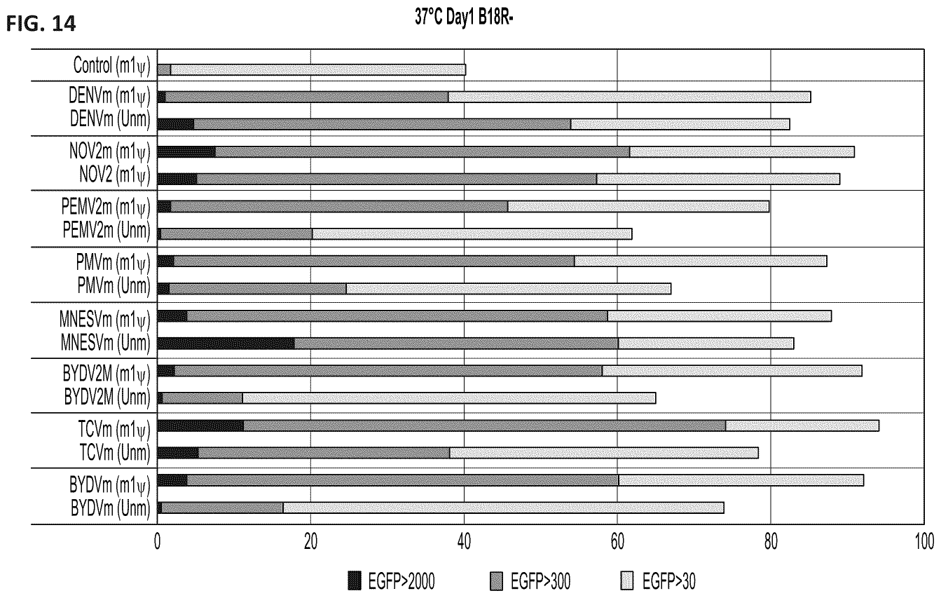

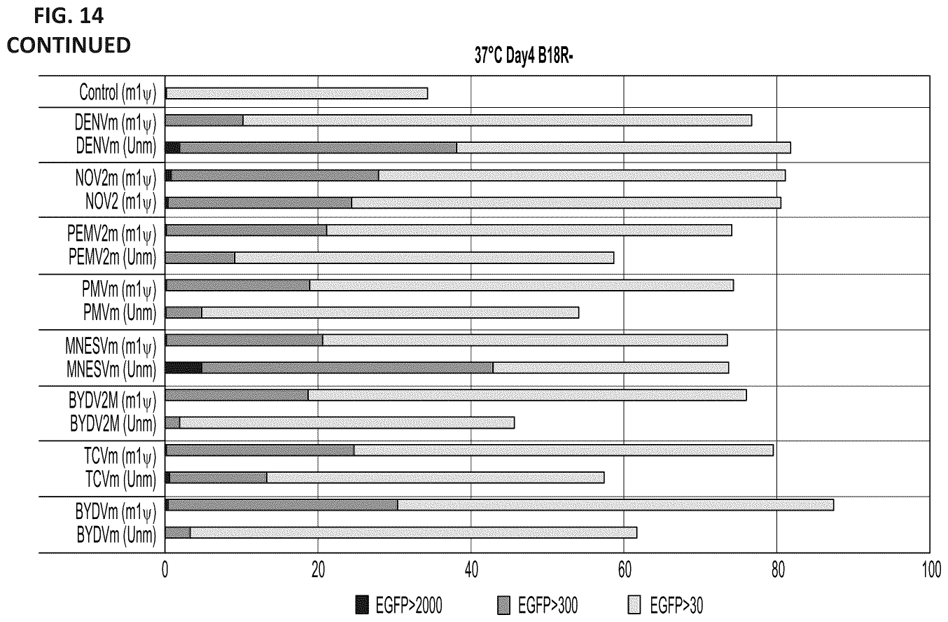

As expected, a poly(A)less Control-EGFP synRNA (5′-Cap1, modified with m1Ψ) showed almost no EGFP expression in all four culture conditions (33° C. or 37° C.; B18R+ or B18R−) on Day 1 and Day 4 (FIG. 11, FIG. 12, FIG. 13, and FIG. 14).

As expected, DENVm-EGFP (unmodified), DENVm-EGFP (modified with m1Ψ), NOV2-EGFP (modified with m1Ψ), and NOV2m-EGFP (modified with mlI) showed strong EGFP expression in all four culture conditions (33° C. or 37° C.; B18R+ or B18R−) on Day 1 and Day 4 (FIG. 11, FIG. 12, FIG. 13, and FIG. 14).

To our surprise, plant +ssRNA virus-based synRNAs with 5′-Cap (both nucleoside unmodified and modified with mlI) showed strong EGFP expression in in all four culture conditions (33° C. or 37° C.; B18R+ or B18R−) on Day 1 and Day 4. Especially, TCVm and MNESVm showed much stronger EGFP expression than others, including DENVm-EGFP, NOV2-EGFP, and NOV2m-EGFP (FIG. 11, FIG. 12, FIG. 13, and FIG. 14).

TCVm showed features that are similar to standard synRNA: Nucleoside modified version (m1Ψ) showed much higher expression of EGFP than unmodified version (FIG. 15 and FIG. 16); the EGFP expression was not influenced by the presence of B18R; the expression was high on Day 1, which decreased over time, but the expression was relatively maintained till Day 4; and the expression was observed at both 33° C. and 37° C. (FIG. 15).

Interestingly, MNESVm showed much higher expression in the nucleoside unmodified form than in the nucleoside modified form (FIG. 15 and FIG. 16). This expression pattern is the same as DENVm synRNA albeit different than observed with commonly used synRNAs. The expression from unmodified MNESVm was not much influenced by the presence of B18R. The expression was strongly on Day 1, whose expression was relatively well maintained till Day 4 (FIG. 15).

In summary, plant +ssRNA virus-based synRNAs, when 5′-Cap is added, can be used as poly(A)less synRNAs. Especially, TCVm and MNESVm can drive very high expression of GOI.

### Example 13. Comparison of EGFP Protein Expression from Poly(A)Less TCVm and Control synRNA with 120 Poly(A) Tail

This example describes fluorescence activated cell sorter (FACS) analyses, comparing EGFP fluorescence intensities among the poly(A)less TCVm-EGFP mRNA, Control-EGFP synRNA with the standard 120 poly(A) tail, and non-transfected control.

### Materials and Methods

Cell Culture. Human Dermal Fibroblasts, neonatal (HDFn) were purchased from ThermoFisher Scientific (Catalog No. C0045C) and cultured according to the manufacturer's instructions.

Production and Transfection of TCVm-EGFP, Control-EGFP synRNA with 120 poly(A) tail. TCVm-EGFP (with 5′-Cap, without poly(A)) was produced as described in Example 12. Control-EGFP synRNA (with 5′-Cap, with 120 poly(A)) was made by a published protocol (Warren et al., 2010; Mandall and Rossi, 2013). The 3′-UTR sequence of Control-EGFP synRNA is identical to the Mus musculus hemoglobin alpha, adult chain 1 (Hba-a1), mRNA (NM_008218.2) and the tail-PCR was used to add 120 poly(A) tail. 3×10\hat{ }4 HDFn cells/well were plated in 24-well plate at day −1. The following day, cells were transfected with 1.0 μg of synRNAs using MessengerMax transfection reagent (ThermoFisher). HDFn cells were cultured at 37° C., with 250 ng/mL of B18R (Sigma) for 16 hrs after synRNA transfection and then harvested for FACS analyses.

FACS analyses: FACS analyses were performed in the standard method. Cells were gated by forward scatter (FSC) and side scatter (SSC), and then geometric mean of fluorescence intensity (MFI) was calculated.

### Results and Conclusions

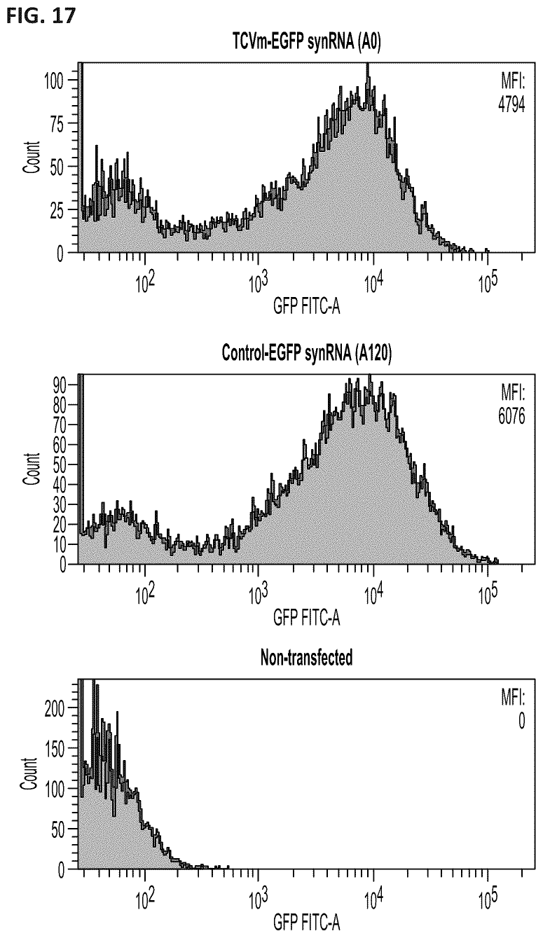

TCVm-EGFP and Control-EGFP showed the GFP intensity of 4,794 (MFI) and 6076 (MFI), respectively (FIG. 17). The result indicates that the translation efficiency of TCVm synRNA, even without a poly(A) tail, is comparable to that of Control-EGFP synRNA with the standard 120 poly(A) tail.

In summary, TCVm, plant +ssRNA virus-based synRNA, when 5′-Cap is added, can be used as poly(A)less synRNAs and can drive very high expression of GOI.

### Example 14. In Vivo Protein Expression from Poly(A)Less +ssRNA Virus-Based synRNAs with 5′-Cap

This example describes the finding that +ssRNA virus-based synRNAs, which lack a poly(A)-tail, can be efficiently translated in vivo, when a 5′-Cap is added.

### Materials and Methods

Mice. C57BL/6 and BALB/c mice were purchased from the Jackson Laboratory, and housed and handled according to a protocol approved by the institutional animal care and use committee (IACUC).

Production ofplasmid DNA for IVT. Because the SapI restriction enzyme site used to linearize the plasmid DNA for IVT was present in luciferase gene, the MluI restriction enzyme was chosen to linearize the plasmid DNA template. Plasmid DNAs described in the Example 1 for NOV2m were modified by adding an MluI restriction enzyme site immediately after the SapI restriction enzyme site. Plasmid DNAs described in the Example 11 for TCVm were modified by adding an MluI restriction enzyme site immediately after the SapI restriction enzyme site. Luciferase gene was cloned into NdeI-NotI sites of the multiple cloning site of these plasmid DNAs.

Production of synRNAs by in vitro transcription. The DNA templates were linearized with MluI restriction enzyme and used for IVT to produce synRNAs: TCVm-LUC2 synRNA (MluI) and NOV2m-LUC2 synRNA (MluI). As a negative control, NOV2m-LUC2 synRNA was also linearized with SapI, which cut the LUC2 gene so that no LUC2 protein is formed (NOV2m-LUC2 synRNA (SapI)). The synRNAs were modified with m1Ψ.

Intramuscular injection of synRNA and luciferase assays. 20.0 μg of synRNAs were complexed with Lipid Nanoparticle (LNP: InvivoFectamine3.0, ThermoFisher) according to the manufacturer's protocol. The synRNA/LNP complexes were directly injected into muscles in the right thigh region of C57BL/6 mice and BALB/c mice. Next day (Day 1) and the following day (Day 2), luciferase activity was monitored by the AMI HTX Bioluminescent Imaging system (Spectral Instruments Imaging, Tucson, AZ).

### Results and Conclusions

Representative bioluminescent images of luciferase assays in mice are shown in FIG. 18A and the luciferase activities assessed by the Bioluminescent Imaging system were plotted in FIG. 18B. The results clearly showed that TCVm-LUC2 (MluI) was highly translated in vivo, even without a poly(A) tail. NOV2m-LUC2 (MluI) also worked, though the translation efficiency was lower than that of TCVm-LUC2. These Poly(A)less synRNAs were translatable in both C57BL/6 and BALB/c.

### Example 15. EGFP Protein Expression from +ssRNA Virus-Based synRNAs with 5′-Cap and 3′-Adenine Homopolymer

This example describes EGFP expression from +ssRNA virus-based synRNAs (with the addition of 5′-Cap and 3′-adenine homopolymer) encoding EGFP. The synRNAs tested include Control-EGFP synRNA, NOV2m-EGFP synRNA, TCVm-EGFP synRNA, and MNESVm-EGFP synRNA.

### Materials and Methods

Cell Culture. Human Dermal Fibroblasts, neonatal (HDFn) were purchased from ThermoFisher Scientific (Catalog No. C0045C) and cultured according to the manufacturer's instructions.

Production of DNA templates by tail-PCR. DNA templates for in vitro transcription (IVT) were generated according to the protocol of Mandal and Rossi (2013). DNA fragments containing a T7 promoter, 5′-UTR, EGFP CDS, and 3′-UTR were amplified from Control-EGFP, NOV2m-EGFP, TCVm-EGFP, and MNESVm-EGFP by the tail-PCR using 3′-primers containing 0 (for A0), 20 (for A20), 30 (for A30), 60 (for A60), and 120 (for A120) thymine (T) nucleotides. Control-EGFP synRNA containing a 3′-UTR of hemoglobin alpha, adult chain 1 (Hba-al) was as described in previous reports (Warren et al., 2010; Mandal and Rossi, 2013). Poly(A)-tail of A120 is a standard length (Warren et al., 2010). NOV2m-EGFP synRNA, TCVm-EGFP synRNA, and MNESVm-EGFP synRNA are as described in the previous sections.

Production of synRNAs by in vitro transcription. The DNA templates were used for IVT to produce synRNAs. Based on the results presented above, NOV2m-EGFP and TCVm-EGFP were modified with m1Ψ, as they performed better compared to nucleoside unmodified version. On the other hand, MNESVm-EGFP was used in the nucleoside unmodified form (Unm), as it performed better than the nucleoside modified form (m1Ψ) in the experiments described previously. A 5′-cap can be added to synRNAs using standard methods. However, for convenience, CleanCapAG (Henderson 2021; Trilink) was used to add a 5′-cap (capI to synRNAs).

Transfection of Control-EGFP synRNA (A0, A20, A30, A60, A120), NOV2m-EGFP synRNA (A0, A20, A30, A60, A120), TCVm-EGFP synRNA (A0, A20, A30, A60, A120), and MNESVm-EGFP synRNA (A0, A20, A30, A60, A120) into HDFn cells. 3×10\hat{ }4 HDFn cells/well were plated in a 24-well plate at day −1. The following day, cells were transfected with 1.0 μg of synRNAs using MessengerMax transfection reagent (ThermoFisher). HDFn cells were cultured at 33° C. or 37° C., with or without 250 ng/mL of B18R (Sigma), for 24 hours after synRNA transfection.

EGFP expression. EGFP expression levels (fluorescence intensities) were measured by Moxi GO II (ORFLO) on Day 1 after transfection. GFP-positive (+) cells were presented as a percent (%) of total cells at the fluorescence intensity of GFP>30 for all GFP+ cells, GFP>300 for strong GFP+ cells, or GFP>2000 for very strong GFP+ cells.

### Results and Conclusions

Control-EGFP synRNAs showed the expression patterns as expected. AG and A20 showed no or very low translation (FIG. 19, 20, 21, 22). Starting from A30, the translation efficiency gradually increased to A60, and to A120. The translation efficiency of Control-EGFP synRNAs was not influenced much by temperature (33° C. or 37° C.), and by the present of B18R (FIG. 19, 20, 21, 22).

NOV2m synRNAs showed the expression from AG. The translation efficiency increased by adding A20 poly(A), and further increased by adding A30, A60, and A120. The translation efficiency of NOV2m synRNAs was not influenced much by temperature (33° C. or 37° C.) and by the presence of B18R (FIG. 19, 20, 21, 22).

TCVm synRNAs showed the strong expression from AG. However, the addition of A20 poly(A) reduced the translation efficiency from A0, which increased to the level of AG only by adding poly(A) longer than A30. The translation efficiency of TCVm synRNAs was not influenced much by the temperature (33° C. or 37° C.) and by the presence of B18R (FIG. 19, 20, 21, 22).

MNESVm synRNAs (Unm) showed the strong expression from AG. However, the addition of A20 and A30 3′-adenine homopolymer reduced the translation efficiency from A0, which increased to the level of AG only by adding 3′-adenine homopolymer longer than A60. The translation efficiency of MNESVm synRNAs was not influenced much by the temperature (33° C. or 37° C.) and by the presence of B18R (FIG. 19, 20, 21, 22).

Without poly(A) tail, TCVm-EGFP synRNA (m1Ψ), and MNESVm-EGFP synRNA (Unm) showed strong EGFP expression, followed by NOV2m-EGFP synRNA (m1Ψ). Control-EGFP synRNA (m1Ψ) showed no or very low EGFP expression. When a homopolymer of adenine was added, NOV2m-EGFP synRNAs showed the best expression levels in any length of 3′-adenine homopolymer—A20, A30, A60, A120 (FIG. 19, 20, 21, 22). Notably, even with the standard A120 3′-adenine homopolymer length, NOV2m synRNAs performed much better than Control synRNA, TCVm synRNA, and MNESVm synRNA. synRNAs are most frequently used at the natural in vivo condition, i.e., at 37° C. body temperature and in the absence of B18R. In this condition, considering the desirable short 3′-adenine homopolymer length, it is worth noting that NOV2m synRNAs with A20 and A30 showed equal or even stronger EGFP expression levels than the Control synRNAs containing the standard A120 3′-adenine homopolymer (FIG. 19, 20, 21, 22).

Accordingly, the present disclosure provides RNA molecules comprising a 5′-untranslated region (5′-UTR), at least one coding sequence (CDS) for at least one protein, and a 3′-untranslated region (3′-UTR), followed by a short adenine homopolymer, wherein the 5′-UTR and 3′-UTR are derived from +ssRNA viruses. These new types of synRNAs containing a short adenine homopolymer can be used to express high levels of a protein of interest, while providing advantages for manufacturing and purification though use of an oligo(dT) column, if necessary.

### Example 16. Expression of a Full-Length Human Dystrophin (DMD) from +ssRNA Virus-Based synRNAs In Vitro

This example describes the successful expression of a large protein, DMD, from +ssRNA virus-based synRNAs in vitro.

### Materials and Methods

Cell Culture. Human Dermal Fibroblasts, neonatal (HDFn) were purchased from ThermoFisher Scientific (Catalog No. C0045C) and cultured according to the manufacturer's instructions.

Production of a plasmid DNA. This example used NOV2m synRNA with a 28 residue 3′-adenine homopolymers (NOV2m-A28). To simplify the synRNA production process, 28 adenines were inserted immediately after the 3′-UTR of NOV2m vector (described in FIG. 1C), followed by digestions with NdeI restriction enzyme site to linearize plasmid DNA. A full-length CDS of human dystrophin (DMD) protein (transcript variant Dp427m, NCBI accession number NM_004006) was cloned into a multiple cloning site of NOV2m-A28 (NOV2m-DMD-A28).

Production of synRNAs by in vitro transcription. The plasmid DNA was linearized with NdeI and used for IVT to produce synRNAs with m1Ψ modification. CleanCapAG (Henderson 2021; Trilink) was used to add a 5′-Cap 1. The size of this NOV2m-DMD-A28 synRNA was 11.3 kb (FIG. 23A).

Transfection of NOV2m-DMD-A28 synRNA. 6×10\hat{ }4 HDFn cells/well were plated in a 4-well chamber slide. The following day, cells were transfected with 1.0 μg or 2.0 μg of NOV2m-DMD-A28 synRNA using MessengerMax transfection reagent (ThermoFisher). HDFn cells were cultured at 37° C. with 250 ng/mL of B18R (Sigma), for 24 hours after synRNA transfection.

Immunohistochemistry. Human DMD protein was detected by immunohistochemistry using an anti-Dystrophin antibody (MANDYS106: Millipore Sigma), which recognizes only human DMD, but not mouse DMD. Immunohistochemistry was performed according to a standard method. To visualize nuclei, samples were also stained with 4′,6-diamidino-2-phenylindole (DAPI).

### Results and Conclusions

A plasmid DNA containing a full-length DMD coding region (transcript variant Dp427m, NCBI accession number NM_004006) and 28 3′-adenine homopolymers was successfully constructed. The plasmid DNA was amplified in E. coli in a standard procedure. The plasmid DNA was linearized with NdeI restriction enzyme digestion and directly used as a DNA template for IVT. Unlike the Example 15 that used a tail-PCR to add 3′-adenine homopolymers to DNA templates, 28 adenine homopolymers were already incorporated into the plasmid DNA. Therefore, DNA template preparation was simpler, more efficient, and lower-cost, compared to the tail-PCR method.

NOV2m-DMD-A28 synRNA (11.3 kb) was successfully produced by the standard IVT method. The nucleotide sequence of NOV2m-DMD-A28 synRNA is set forth as SEQ ID NO:42.

After the delivery into HDFn cells by a standard transfection method, NOV2m-DMD-A28 synRNA produced DMD, which was detected by the immunohistochemistry using an antibody against human DMD (MANDYS106) (FIG. 23B). The transfection efficiency was high and the expression of DMD was strong (FIG. 23B).

Accordingly, the present disclosure demonstrates that a large protein (e.g., full-length human DMD) can be expressed from +ssRNA virus-based synRNAs in vitro. The present disclosure also demonstrates that an entire synRNA can be encoded in a plasmid DNA, thereby making the synRNA production process simple, efficient, and cost-effective.

### Example 17. Expression of a Full-Length Human Dystrophin (DMD) from +ssRNA Virus-Based synRNAs In Vivo

This example describes the successful expression of a large protein, DMD, from +ssRNA virus-based synRNAs in vivo.

### Materials and Methods

Mice. BALB/c mice were purchased from the Jackson Laboratory, and housed and handled according to a protocol approved by the institutional animal care and use committee (IACUC).

Production of a plasmid DNA. This example used NOV2m synRNA with a 28 residue 3′-adenine homopolymer (NOV2m-A28). To simplify the synRNA production process, 28 adenines were inserted immediately after the 3′-UTR of NOV2m vector (described in FIG. 1C), followed by digestions with NdeI restriction enzyme. A DNA encoding a fusion protein of a luciferase (LUC) and a full-length human dystrophin (DMD) protein (transcript variant Dp427m, NCBI accession number NM_004006) was cloned into a multiple cloning site of NOV2m-A28 (NOV2m-LUC-DMD-A28).

Production of synRNAs by in vitro transcription. The plasmid DNA was linearized with NdeI and used for IVT to produce synRNAs with m1Ψ modification. CleanCapAG (Henderson 2021; Trilink) was used to add a 5′-Cap 1. The size of this NOV2m-LUC-DMD-A28 synRNA was 13.0 kb (FIG. 24A).

Intramuscular injection of synRNA and luciferase assays. 20.0 μg of NOV2m-LUC-DMD-A28 synRNA was complexed with Lipid Nanoparticle (LNP: InvivoFectamine3.0, ThermoFisher) according to the manufacturer's protocol. The synRNA/LNP complexes were directly injected into muscles in the right thigh region of BALB/c mice (Day 0). The following day (Day 1), luciferase activity was monitored by the AMI HTX Bioluminescent Imaging system (Spectral Instruments Imaging, Tucson, AZ).

### Results and Conclusions

Representative bioluminescent images of mice to visualize luciferase activity are shown in FIG. 24B. Luciferase activity assessed by the Bioluminescent Imaging system were plotted in FIG. 24C. The results clearly showed that NOV2m-LUC-DMD-A28 synRNA was translated, resulting in the production a fusion protein of a LUC and a full-length DMD in vivo.

The present disclosure demonstrates that a large fusion protein can be expressed from +ssRNA virus-based synRNAs in vivo. The present disclosure also demonstrates that an entire synRNA can be encoded in a plasmid DNA, thereby making the synRNA production process simple, efficient, and cost-effective.

### Example 18. Expression of a Full-Length Human Collagen Type VII Alpha-1 (VII) Chain (COL7A1) Protein from +ssRNA Virus-Based synRNAs In Vitro

This example describes the successful expression of a large protein, COL7A1, from +ssRNA virus-based synRNAs in vitro.

### Materials and Methods

Cell Culture. Human Dermal Fibroblasts, neonatal (HDFn) were purchased from ThermoFisher Scientific (Catalog No. C0045C) and cultured according to the manufacturer's instructions.

Production of a plasmid DNA. This example used NOV2m synRNA with a 28 residue 3′-adenine homopolymer (NOV2m-A28). To simplify the synRNA production process, 28 adenines were inserted immediately after the 3′-UTR of NOV2m vector (described in FIG. 1C), followed by digestions with NdeI restriction enzyme site to linearize a plasmid DNA. A full-length CDS of human collagen type VII alpha-1 (VII) chain (COL7A1) protein (NCBI accession number NM_000094) was cloned into a multiple cloning site of NOV2m-A28 (NOV2m-COL7A1-A28). A fusion protein of a full-length CDS of COL7A1 protein (NCBI accession number NM_000094) and a luciferase (LUC2) protein was also cloned into a multiple cloning site of NOV2m-A28 (NOV2m-COL7A1-LUC-A28).

Production of synRNAs by in vitro transcription. The plasmid DNAs were linearized with NdeI and used for IVT to produce synRNAs with m1Ψ modification. CleanCapAG (Henderson 2021; Trilink) was used to add a 5′-Cap 1. The size of the NOV2m-DMD-A28 synRNA was 9.0 kb, and the size of the NOV2m-COL7A1-LUC-A28 was 10.7 kb (FIG. 25A).

Transfection of NOV2m-COL7A1-A28 and NOV2m-COL7A1-LUC-A28 synRNA. 6×10\hat{ }4 HDFn cells/well were plated in a 4-well chamber slide. The following day, cells were transfected with 1.0 μg of NOV2m-COL7A1-A28 synRNA or NOV2m-COL7A1-LUC-A28 synRNA using MessengerMax transfection reagent (ThermoFisher). In one condition, the transfection was performed only once (lx transfection). In another condition, the transfection was repeated on the 2^{nd} day and the 3^{rd} day (3× transfection). HDFn cells were cultured at 37° C. with 250 ng/mL of B18R (Sigma), for 24 hours after synRNA transfection (lx transfection) or for 24 hours after the third synRNA transfection (3× transfection).

Immunohistochemistry. Expression of human COL7A1 protein was detected by immunohistochemistry using anti-COL7A1 antibody (MCA597GA), which recognizes only human COL7A1, but not mouse COL7A1. Immunohistochemistry was performed according to a standard method. To visualize nuclei, samples were also stained with 4′,6-diamidino-2-phenylindole (DAPI).

### Results and Conclusions

A plasmid DNA contained a full-length CDS of human collagen type VII alpha-1 (VII) chain (COL7A1) protein (NCBI accession number NM_000094) and a 28 residue 3′-adenine homopolymer was successfully constructed. The plasmid DNA was amplified in E. coli in the standard procedure. The plasmid DNA was linearized with NdeI restriction enzyme digestion and directly used as a DNA template for IVT. Unlike the Example 15 that used a tail-PCR to add a 3′-adenine homopolymer to DNA templates, the 28 adenine homopolymer was incorporated into the plasmid DNA. Therefore, DNA template preparation was simpler, more efficient, and lower-cost, compared to the tail-PCR method.

Both NOV2m-COL7A1-A28 synRNA (9.0 kb) and NOV2m-COL7A1-LUC-A28 synRNA (10.7 kb) were successfully produced.

After delivery into HDFn cells by a standard transfection method, both NOV2m-COL7A1-A28 synRNA and NOV2m-COL7A1-LUC-A28 synRNA produced COL7A1 protein and COL7A1-LUC fusion protein, respectively, as detected by immunohistochemistry using an antibody against human COL7A1 (FIG. 25B). The transfection efficiency was high and the expression of DMD was strong (FIG. 25B).

Accordingly, the present disclosure demonstrates that a large protein (e.g., full-length human COL7A1), as well as an even larger fusion protein, can be expressed from +ssRNA virus-based synRNAs vector in vitro. The present disclosure also demonstrates that an entire synRNA can be encoded in a plasmid DNA, thereby making the synRNA production process simple, efficient, and cost-effective.

### Example 19. EGFP Protein Expression from DENVm synRNA with 5′-Cap and 3′-Adenine Homopolymer

This example describes EGFP expression from DENVm synRNA (with the addition of 5′-Cap and 3′-adenine homopolymer) encoding EGFP.

### Materials and Methods

Cell Culture. Human Dermal Fibroblasts, neonatal (HDFn) were purchased from ThermoFisher Scientific (Catalog No. C0045C) and cultured according to the manufacturer's instructions.

Production of DNA templates by tail-PCR. DNA templates for in vitro transcription (IVT) were generated according to the protocol of Mandal and Rossi (2013). DNA fragments containing a T7 promoter, 5′-UTR, EGFP CDS, and 3′-UTR were amplified from DENVm-EGFP by the tail-PCR using 3′-primers containing 0 (for A0), 20 (for A20), 30 (for A30), 60 (for A60), and 120 (for A120) thymine (T) nucleotides. DENVm-EGFP synRNA are as described in the previous sections.

Production of synRNAs by in vitro transcription. The DNA templates were used for IVT to produce synRNAs.

Based on the results presented above, DENVm-EGFP synRNA was used in the nucleoside unmodified form (Unm). A 5′-cap was added to synRNAs using standard methods. However, for convenience, CleanCapAG (Henderson 2021; Trilink) was used to add a 5′-cap (cap1 to synRNAs).

Transfection of DENVm-EGFP synRNA (A0, A20, A30, A60, A120) into HDFn cells. 3×10\hat{ }4 HDFn cells/well were plated in a 24-well plate at day −1. The following day, cells were transfected with 1.0 μg of synRNAs using MessengerMax transfection reagent (ThermoFisher). HDFn cells were cultured at 33° C. or 37° C., with or without 250 ng/mL of B18R (Sigma), for 24 hours after synRNA transfection.

EGFP expression. EGFP expression levels (fluorescence intensities) were measured by Moxi GO II (ORFLO) on Day 1 after transfection. GFP-positive (+) cells were presented as a percent (%) of total cells at the fluorescence intensity of GFP>30 for all GFP+ cells, GFP>300 for strong GFP+ cells, or GFP>2000 for very strong GFP+ cells.

### Results and Conclusions

DENVm synRNAs in nucleoside unmodified forms (Umn) showed the expression from AG in vitro. The translation efficiency increased by adding A20 poly(A), and further increased by adding A30, A60, and A120. The translation efficiency of DENVm synRNAs (Unm) was not influenced very much by temperature (33° C. or 37° C.) or the presence of B18R (FIG. 26).

Accordingly, the present disclosure provides RNA molecules comprising a 5′-untranslated region (5′-UTR), at least one coding sequence (CDS) for at least one protein, and a 3′-untranslated region (3′-UTR), followed by a short adenine homopolymer, wherein the 5′-UTR and 3′-UTR are derived from Dengue virus. Although Dengue viruses do not naturally have a polyA at their 3′-ends, the current finding demonstrates that these new types of synRNAs containing a short adenine homopolymer can be used to express high levels of a protein of interest, while providing advantages for manufacturing and purification though use of an oligo(dT) column, if necessary.

### Example 20. EGFP Protein Expression from synRNAs with 5′-Cap, 3′-Adenine Homopolymer, and 5′-UTR and 3′-UTR Derived from SARS-CoV-2 Viruses

This example describes EGFP expression from SARS-CoV-2 virus-based synRNAs encoding EGFP.

### Materials and Methods

Cell Culture. Human Dermal Fibroblasts, neonatal (HDFn) were purchased from ThermoFisher Scientific (Catalog No. C0045C) and cultured according to the manufacturer's instructions.

Production of DNA template. A plasmid DNA containing a T7 promoter, 5′-UTR (from SARS-CoV-2), MCS, 3′-UTR (from SARS-CoV-2), and 50 adenine homopolymers at the 3′end was synthesized (FIG. 27A). The EGFP coding region (SEQ ID NO:5) was cloned into the MCS (SEQ ID NO:8).

Production of synRNAs by in vitro transcription. The DNA template was used for IVT to produce synRNAs named SARSVm. The nucleotide sequence of the 5′ UTR of SARSVm is set forth as SEQ ID NO:37 and the nucleotide sequence of the 3′-UTR and adenine homopolymer is set forth as SEQ ID NO:38. Both nucleoside-unmodified synRNA (Unm) and nucleoside-modified synRNA (m1Ψ) were synthesized and tested. A 5′-cap was added to synRNAs using standard methods. However, for convenience, CleanCapAU (Henderson 2021; Trilink) was used to add a 5′-cap (cap1 to synRNAs).

Transfection of SARSVm-EGFP into HDFn cells. 3×10\hat{ }4 HDFn cells/well were plated in a 24-well plate at Day −1. The following day (Day 0), cells were transfected with 1.0 μg of synRNAs using MessengerMax transfection reagent (ThermoFisher). HDFn cells were cultured at 33° C. or 37° C., with or without 250 ng/mL of B18R (Sigma), for 24 hours after synRNA transfection.

EGFP expression. EGFP expression levels (fluorescence intensities) were measured by Moxi GO II (ORFLO) on Day 1 after transfection. GFP-positive (+) cells were presented as a percent (%) of total cells at the fluorescence intensity of GFP>30 for all GFP+ cells, GFP>300 for strong GFP+ cells, or GFP>2000 for very strong GFP+ cells.

### Results and Conclusions

SARSVm synRNAs in nucleoside-modified form (m1Ψ) showed strong expression in both temperature conditions (33° C. or 37° C.) and in both B18R (+) and B18R (−) conditions (FIG. 27B). However, in nucleoside-unmodified form (Unm), the protein translation efficiency was low in all four conditions [33° C. or 37° C.; B18R (+) or B18R (−)](FIG. 27B).

Accordingly, the present disclosure provides RNA molecules comprising a 5′-untranslated region (5′-UTR), at least one coding sequence (CDS) for at least one protein, and a 3′-untranslated region (3′-UTR), followed by a short adenine homopolymer, wherein the 5′-UTR and 3′-UTR are derived from +ssRNA viruses, which naturally possess adenine homopolymers at their 3′ends.

### Example 21. In Vivo Protein Expression From NOV2m (A50), NOV2m (A30), DENVm (A50) and DENVm (A30) in Muscle and Skin

This example describes the finding that +ssRNA virus-based synRNAs with short adenine homopolymers can be efficiently translated in mouse muscles and skins.

### Materials and Methods

Mice. BALB/c mice were purchased from the Jackson Laboratory, and housed and handled according to a protocol approved by the institutional animal care and use committee (IACUC).

Production of plasmid DNAs for IVT. A NOV2m plasmid DNA was modified to include 50 adenine homopolymers after 3′-UTR, followed by NdeI restriction enzyme site for a plasmid linearization. A DENVm plasmid DNA was modified to include 50 adenine homopolymers after 3′-UTR, followed by NdeI restriction enzyme site for a plasmid linearization. Luciferase gene was cloned into MCS of both plasmid vectors.

Production of synRNAs by in vitro transcription. The DNA templates were linearized with NdeI restriction enzyme and used for IVT to produce synRNAs: NOV2m (A50)-LUC synRNA and DENVm (A50)-LUC. Both nucleoside-unmodified synRNA (Unm) and nucleoside-modified synRNA (m1Ψ) were synthesized and tested. A 5′-cap was added to synRNAs using standard methods. However, for convenience, CleanCapAG (Henderson 2021; Trilink) was used to add a 5′-cap (cap1 to synRNAs).

Intramuscular injection of synRNA and luciferase assays. 20.0 g of synRNAs were complexed with Lipid Nanoparticle (LNP: InvivoFectamine3.0, ThermoFisher) according to the manufacturer's protocol. The synRNA/LNP complexes were directly injected into muscles in BALB/c mice. Starting from next day (Day 1), luciferase activity was monitored by the AMI HTX Bioluminescent Imaging system (Spectral Instruments Imaging, Tucson, AZ). Monitoring of luciferase activities was stopped on Day 8, when the signals could not be detected any more.

Intradermal injection of synRNA and luciferase assays. 20.0 g of synRNAs were dissolved in lactated Ringer's solution. To test the effects of chitosan oligosaccharide on gene expression, synRNAs were mixed with or without Chitosan oligosaccharides (1.5 μg/ml final concentration). Final volume was 60 μL. Starting from next day (Day 1), luciferase activity was monitored by the AMI HTX Bioluminescent Imaging system (Spectral Instruments Imaging, Tucson, AZ). Monitoring of luciferase activities was stopped on Day 13, when the signals could not be detected any more.

### Results and Conclusions

Muscle: Luciferase activity as a consequence of intramuscular injection of synRNAs was assessed by the Bioluminescent Imaging system and results were plotted in FIG. 28. The results showed that all NOV2m (A50), NOV2m (A30), DENVm (A50) and DENVm (A30) were translated at high levels in vivo. Although the difference was not large, the 50 adenine homopolymers worked better than 30 adenine homopolymers. Nucleoside modification helped to increase the translation efficiency in muscle for NOV2m, but not for DENVm. Overall, DENVm (A50), either modified or unmodified, performed better than NOV2m (A50).

Skin: Luciferase activity as a consequence of intradermal injection of synRNAs was assessed by the Bioluminescent Imaging system and results were plotted in FIG. 29. The results showed that all NOV2m (A50), NOV2m (A30), DENVm (A50) and DENVm (A30) were translated at high levels in vivo. However, the 50 adenine homopolymers worked better than 30 adenine homopolymers for both NOV2m and DENVm. Nucleoside modification did not make much of a differences, but the modified synRNA worked better than unmodified synRNA for NOV2m, whereas the unmodified synRNA worked better than modified synRNA for DENVm. Interestingly, Chitosan oligosaccharides dramatically enhances luciferase expression under all of the tested conditions: NOV2m (m1Ψ), NOV2m (Unm), DENVm (m1Ψ), and DENVm (Unm). Overall, NOV2m (A50, m1Ψ) worked better than others, but DENVm (A50, Unm) performed at a comparable level.

### Example 22. Expression of a Full-Length Human Dystrophin (DMD) from +ssRNA Virus-Based synRNAs In Vivo

This example describes the successful expression of a large protein, a full-length human dystrophin, from +ssRNA virus-based synRNAs in vivo.

### Materials and Methods

Mice: BALB/c mice were purchased from the Jackson Laboratory, and housed and handled according to a protocol approved by the institutional animal care and use committee (IACUC).

Production of a plasmid DNA. This example used NOV2m synRNA with a 28 residue 3′-adenine homopolymers (NOV2m-A28). To simplify the synRNA production process, 28 adenines were inserted immediately after the 3′-UTR of NOV2m vector (described in FIG. 1C), followed by digestions with NdeI restriction enzyme site to linearize plasmid DNA. A full-length CDS of human dystrophin (DMD) gene (transcript variant Dp427m, NCBI accession number NM_004006) was cloned into a multiple cloning site of NOV2m-A28 (NOV2m-DMD-A28).

Production of synRNAs by in vitro transcription. The plasmid DNA was linearized with NdeI and used for IVT to produce synRNAs with m1Ψ modification. CleanCapAG (Henderson 2021; Trilink) was used to add a 5′-Cap 1. The size of this NOV2m-DMD-A28 synRNA was 11.3 kb (FIG. 23A).

Intramuscular injection of synRNA. 20.0 μg of NOV2m-DMD-A28 synRNA was complexed with InvivoFectamine3.0 (ThermoFisher) according to the manufacturer's protocol. The synRNA/Invivofectamine complexes were directly injected into muscles of BALB/c mice (Day 0). One day after the injection, the mice were sacrificed, and the skeletal muscle of injection sites was dissected for immunostaining.

Immunohistochemistry. Human DMD protein was detected by immunohistochemistry using an anti-Dystrophin antibody (MANDYS106: Millipore Sigma), which recognizes only human DMD, but not mouse DMD. Mouse and human DMD proteins were detected by immunohistochemistry using anti-Dystrophin antibody (AB15277: Abcam), which recognizes both mouse and human DMD. Immunohistochemistry was performed according to a standard method. To visualize nuclei, samples were also stained with 4′,6-diamidino-2-phenylindole (DAPI). Con-focal microscopic images were taken.

### Results and Conclusions

Representative immunostaining images are shown in FIG. 31. Muscle sections were stained with anti-human DMD antibody (MANDYS106) (upper panel), which does not recognize mouse dystrophin but recognizes human dystrophin. The no treatment muscle did not show any staining, but the NOV2m-DMD-A28 synRNA injected muscle (two representative images) showed the production and proper localization of human dystrophin protein (upper panel). In addition, muscle sections stained with anti-mouse DMD antibody (AB15277), which recognizes both mouse and human dystrophin proteins, showed the proper localization of mouse dystrophin (and also human dystrophin) proteins in both no treatment muscle and the NOV2m-DMD-A28 synRNA injected muscle (lower panel).

The present disclosure demonstrates that a large protein such as a full-length human dystrophin protein can be expressed from +ssRNA virus-based synRNAs in vivo. The produced dystrophin protein can be properly localized in mouse skeletal muscles.

### Example 23. Recovery of Muscle Strength in Mutant Mice by Intramuscular Injection of +ssRNA Virus-Based synRNA Encoding a Human Dystrophin Protein

This example describes the successful functional recovery of skeletal muscles mutant mice that lacks mouse dystrophin protein by the intramuscular injection of +ssRNA virus-based synRNAs encoding a full-length human dystrophin protein.

### Materials and Methods

Mice: D2.mdx mice, also known as D2.B10-Dmd^{mdx}/J mice, were purchased from the Jackson Laboratory. Per the Jackson Laboratory website, “The D2.B10 (DBA/2-congenic) Dmd^{mdx} mouse (also referred to as DBA/2J-mdx or D2-mdx mice) may be a superior Duchenne muscular dystrophy model as it better recapitulates several of the human characteristics of DMD myopathology (lower hind limb muscle weight, fewer myofibers, increased fibrosis and fat accumulation, and muscle weakness) relative to strains with this mutant allele on other genetic backgrounds”. DBA/2 mice (wildtype control, recommended by the Jackson Laboratory) were also purchased from the Jackson Laboratory. Mice were housed and handled according to a protocol approved by the institutional animal care and use committee (IACUC).

Production of a plasmid DNA. This example used NOV2m synRNA with a 28 residue 3′-adenine homopolymers (NOV2m-A28). To simplify the synRNA production process, 28 adenines were inserted immediately after the 3′-UTR of NOV2m vector (described in FIG. 1C), followed by digestions with NdeI restriction enzyme site to linearize plasmid DNA. A full-length CDS of human dystrophin (DMD) gene (transcript variant Dp427m, NCBI accession number NM_004006) was cloned into a multiple cloning site of NOV2m-A28 (NOV2m-DMD-A28).

Production of synRNAs by in vitro transcription. The plasmid DNA was linearized with NdeI and used for IVT to produce synRNAs with m1Ψ modification. CleanCapAG (Henderson 2021; Trilink) was used to add a 5′-Cap 1. The size of this NOV2m-DMD-A28 synRNA was 11.3 kb (FIG. 23A).

Intramuscular injection of synRNA. 20.0 μg of NOV2m-DMD-A28 synRNA was complexed with InvivoFectamine3.0 (ThermoFisher) according to the manufacturer's protocol. D2.mdx mutant mice received 3 intramuscular injections in the ventral forearm and 2 intramuscular injections in the dorsal forearm with approximately 4 μg (12 μL) of NOV2m-DMD-A28 synRNA using 31G needles: a total of 20 μg (in 60 μL) each for the right forearm and the left forearm.

Muscle strength measurement: Peak muscle strength of the forearm was measured by a grip strength meter (Harvard apparatus). The measurement was performed twice, 30 minutes apart. Peak muscle strength was normalized by mouse weight, and the average of two measurements was used for the analyses.

### Results and Conclusions

D2.mdx mutant mice (Coley, Bogdanik et al. 2016, Hammers, Hart et al. 2020) and wild type DBA/2 mice received 3 intramuscular injections in the ventral forearm and 2 intramuscular injections in the dorsal forearm with approximately 4 μg (12 μL) of NOV2m-DMD-A28 synRNA using 31G needles: a total of 20 μg (in 60 μL) each for the right forearm and the left forearm. The injections started at week 11 and continued once per week for a total of 6 injections. The final injection was at 16 weeks of age. One week after the final injection (measured at 17 weeks of age), peak muscle strength of the forearm was measured by a grip strength meter. The measurement was performed twice, 30 minutes apart. Peak muscle strength was normalized by mouse weight, and the average of two measurements was used for the analyses. FIG. 32A shows peak muscle strength one week after the final injection (measured at 17 weeks of age). The NOV2m-DMD-A28 synRNA injected group showed statistically significant (* p<0.05) recovery of muscle strength compared to the non-injected group (D2.mdx) and a control mRNA-LUC injected group (D2.mdx-LUC). There was no statistically significant difference between the D2.mdx-DMD and wild type DBA/2 groups. For the NOV2m-DMD-A28 synRNA injected group, there were no safety findings associated with either the injection or the treatment.

We also tested whether a single intramuscular injection of NOV2m-DMD-A28 synRNA could recover muscle strength in D2.mdx mutant mice. To minimize the damage to muscle tissues by a 31G needle (0.261 mm outside diameter), we used a 34G needle (0.159 mm outside diameter). 20 μg of NOV2m-DMD-A28 synRNA was mixed with Invivofactamine (ThermoFisher) in a total volume of 60 μL. D2.mdx mutant mice received a single intramuscular injection in the ventral forearm at 3 sites and in the dorsal forearm at 2 sites of 4 μg (12 μL) of mRNA-DMD using 34G needles: a total of 20 μg (in 60 μL) each for the right forearm and the left forearm. The injection was performed at 18 weeks of age. Three weeks later, at 21 weeks of age, peak muscle strength of the forearm was measured by a grip strength meter (Harvard apparatus). The measurement was performed twice, 30 minutes apart. Peak muscle strength was normalized by mouse weight, and the average of two measurements was used for the analyses.

As shown in FIG. 32B, the NOV2m-DMD-A28 synRNA injected group (n=5) showed recovery of muscle strength, whereas the non-injected control group (n=4) did not. This indicates that a single NOV2m-DMD-A28 synRNA injection not only restores the muscle strength in D2.mdx mutant mice but also that the produced dystrophin proteins were stable and remained in the injection site for at least 3 weeks.

### REFERENCES

Ball et al., (1992). Replication of Nodamura Virus after Transfection of Viral RNA into Mammalian Cells in Culture. Journal Of Virology, 66(4):2326-2334.
Coley et al., (2016). Effect of genetic background on the dystrophic phenotype in mdx mice. Human Molecular Genetics, 25(1):130-145.
Dorrington et al., (2009). ICTV Virus Taxonomy Profile: Tetraviridae.
Fang et al., (2022). Advances in COVID-19 mRNA vaccine development. Signal Transduction and Targeted Therapy, 7(1):94.
Hammers et al., (2020). The D2.mdx mouse as a preclinical model of the skeletal muscle pathology associated with Duchenne muscular dystrophy. Scientific Reports, 10:14070.
Henderson et al., (2021). Cap 1 Messenger RNA Synthesis with Co-transcriptional CleanCap® Analog by In Vitro Transcription. Current Protocols, 1(2):e39.
Holtkamp et al., (2006). Modification of antigen-encoding RNA increases stability, translational efficacy, and T-cell stimulatory capacity of dendritic cells. Blood, 108(13): 4009-4017.
Jackson et al., (2020). The promise of mRNA vaccines: a biotech and industrial perspective. npj Vaccines, 5(1).
Johnson et al., (2003). Recovery of infectivity from cDNA clones of Nodamura virus and identification of small nonstructural proteins. Virology, 305(2): 436-451.
Kinney et al., (1997). Construction of infectious cDNA clones for dengue 2 virus: strain 16681 and its attenuated vaccine derivative, strain pdk-53. Virology, 230(2):300-308.
Kowalski et al., (2019). Delivering the Messenger: Advances in Technologies for Therapeutic mRNA Delivery. Molecular Therapy, 27(4): 710-728.
Mandal and Rossi (2013). Reprogramming human fibroblasts to pluripotency using modified mRNA.” Nat Protoc 8(3): 568-582.
Mazeaud et al., (2018). The multiples fates of the flavivirus RNA genome during pathogenesis. Front Genet. 9:595. Mencin et al., (2023). Development and scale-up of oligo-dt monolithic chromatographic column for mRNA capture through understanding of base-pairing interactions. Separation and purification technology 2023 vol. 304.
Newman and Brown, (1976). Absence of poly (A) from the infective RNA of Nodamura virus. Journal of General Virology, 30(1): 137-140.
Nicholson and Pasquinelli, (2019). Tales of Detailed Poly(A) Tails. Trends in Cell Biology, 29(3): 191-200.
Nicholson and White (2011). Y cap-independent translation enhancers of positive-strand RNA plant viruses. Curr Opin Virol. 1(5):373-80.
Reuter and Mathews (2010). RNAstructure: software for RNA secondary structure prediction and analysis. BMC Bioinformatics 11:129.
Rosskopf et al., (2010). A 3′ terminal stem-loop structure in Nodamura virus RNA2 forms an essential cis-acting signal for RNA replication. Virus Research, 150(1-2): 12-21.
Sachs et al., (1987). A single domain of yeast poly(A)-binding protein is necessary and sufficient for RNA binding and cell viability. Mol Cell Biol, 7(9): 3268-76.
Sahul et al., (2019). ICTV virus taxonomy profile: Nodaviridae. Journal of General Virology, 100(1): 3-4.
Simmonds et al., (2017): ICTV Virus Taxonomy Profile: Flaviviridae, Journal of General Virology, 98:2-3.
Trepotec et al., (2019). Segmented poly(A) tails significantly reduce recombination of plasmid DNA without affecting mRNA translation efficiency or half-life. RNA, 25(4): 507-518.
Vavilis et al. (2023). mRNA in the context of protein replacement therapy. Pharmaceutics. 15(1): 166.Wadhwa et al., (2020). Opportunities and challenges in the delivery of mRNA-based vaccines. Pharmaceutics, 12(2).
Warren et al., (2010). Highly Efficient Reprogramming to Pluripotency and Directed Differentiation of Human Cells with Synthetic Modified mRNA. Cell Stem Cell, 7(5): 618-630.

1-48. (canceled)

49. An RNA molecule comprising from 5′ to 3′: a 5′-untranslated region (5′-UTR), at least one coding sequence (CDS) for at least one protein, and a 3′-untranslated region (3′-UTR), wherein
the 3′-UTR comprises a virus 3′-UTR or fragment thereof, wherein the fragment is at least 40 nucleotides in length,
the CDS encodes a full-length human dystrophin or a full-length human collagen type VII alpha-1 (VII) chain (COL7A1) protein, and replaces at least a portion of an open reading frame of the virus, and
the virus is a positive-sense, single-stranded RNA (+ssRNA) virus.

50. The RNA molecule of claim 49, wherein the genome of the virus lacks a poly(A) tail.

51. The RNA molecule of claim 50, further comprising a heterologous adenine homopolymer at its 3′ end, optionally wherein the adenine homopolymer is from 20 to 60 nucleotides in length.

52. The RNA molecule of claim 49, wherein the genome of the virus comprises a poly(A) tail, and the RNA molecule further comprises the poly(A) tail.

53. The RNA molecule of claim 52, further comprising a heterologous adenine homopolymer at the 3′ end of the poly(A) tail, optionally wherein the poly(A) tail and the heterologous adenine homopolymer together are between 20 and 120 nucleotides in length.

54. An RNA molecule comprising from 5′ to 3′: a 5′-untranslated region (5′-UTR), at least one coding sequence (CDS) for at least one protein, and a 3′-untranslated region (3′-UTR), wherein
the 5′-UTR is a 5′-UTR of a Dengue virus or fragment thereof, and the 3′-UTR is a 3′-UTR of the Dengue virus or fragment thereof, wherein the fragment is at least 40 nucleotides in length and
the CDS is heterologous to the Dengue virus and replaces an open reading frame of the Dengue virus such that a portion of the open reading frame that interacts with a complementary sequence of the 3′-UTR to form a circular conformation remains in the RNA molecule.

55. The RNA molecule of claim 54, wherein a first start codon in the portion of the open reading frame remaining in the RNA molecule is mutated so as not to start translation of a first corresponding genome sequence, and a first corresponding portion of the 3′-UTR that interacts with the first start codon to form the circular conformation is mutated so that the mutated first start codon remains complementary to the mutated first corresponding portion of the 3′-UTR to form the circular conformation.

56. The RNA molecule of claim 55, wherein a second start codon in the portion of the open reading frame remaining in the RNA molecule is mutated so as not to start translation of a second corresponding genome sequence, and a second corresponding portion of the 3′-UTR that interacts with the second start codon to form the circular conformation is mutated so that the mutated second start codon remains complementary to the mutated second corresponding portion of the 3′-UTR to form the circular conformation.

57. The RNA molecule of claim 54, further comprising a 5′-cap.

58. The RNA molecule of claim 54, wherein:
(i) the 5′-UTR comprises the nucleotide sequence of SEQ ID NO:25 or a nucleotide sequence having at least 90%, 95%, 96%, 97%, 98% or 99% identity to SEQ ID NO:25; and
(ii) the 3′-UTR comprises the nucleotide sequence of SEQ ID NO:26 or a nucleotide sequence having at least 90%, 95%, 96%, 97%, 98% or 99% identity to SEQ ID NO:26.

59. The RNA molecule of claim 54, further comprising a homopolymer of adenine downstream of the 3′-UTR.

60. The RNA molecule of claim 59, wherein the adenine homopolymer is between about 30 and about 60 nucleotides in length.

61-97. (canceled)

98. The RNA molecule of claim 49, wherein the portion of an open reading frame of the virus that is replaced by the CDS is at least a portion of an RNA-dependent RNA polymerase of the virus.

99. The RNA molecule of claim 50, wherein the virus is a Dengue virus.

100. The RNA molecule of claim 99, wherein:
(i) the 5′-UTR comprises the nucleotide sequence of SEQ ID NO:25 or a nucleotide sequence having at least 90%, 95%, 96%, 97%, 98% or 99% identity to SEQ ID NO:25; and
(ii) the 3′-UTR comprises the nucleotide sequence of SEQ ID NO:26 or a nucleotide sequence having at least 90%, 95%, 96%, 97%, 98% or 99% identity to SEQ ID NO:26.

101. The RNA molecule of claim 99, further comprising a homopolymer of adenine downstream of the 3′-UTR.

102. The RNA molecule of claim 101, wherein the adenine homopolymer is between about 30 and about 60 nucleotides in length.

103. The RNA molecule of claim 49, comprising at least one modified nucleoside.

104. The RNA molecule of claim 54, wherein the portion of an open reading frame of the Dengue virus that is replaced by the CDS is at least a portion of an RNA-dependent RNA polymerase of the Dengue virus.

105. The RNA molecule of claim 54, comprising at least one modified nucleoside.


SEQUENCE LISTING (42 sequences)

SEQ ID NO: 1 - RNA, 788 nt, synthetic construct
  misc_feature 1..788: note = NOV1-EGFP RNA
  source 1..788: mol_type = other RNA
gtattgaatccaaaactcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgattcatcgtcccatctgacgaaacccgaactaggcttatgccagtggt

SEQ ID NO: 2 - RNA, 856 nt, synthetic construct
  misc_feature 1..856: note = NOV2-EGFP RNA
  source 1..856: mol_type = other RNA
gtaaacaaccaataacatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgattccacccacagaagcgttgacgacgcaaaacgtccttaaagcgttgacgacgcaaaacgtccccaagctcgtagcaccgaccctatacccatctctagggtcttcaacctcttggt

SEQ ID NO: 3 - RNA, 179 nt, synthetic construct
  misc_feature 1..179: note = NOV2M RNA, MULTIPLE CLONING SITE
  source 1..179: mol_type = other RNA
gtaaacaaccaataacatcggcgcgccctcagcatcgattcaattgggccggccgcggccgcttccacccacagaagcgttgacgacgcaaaacgtccttaaagcgttgacgacgcaaaacgtccccaagctcgtagcaccgaccctatacccatctctagggtcttcaacctcttggt

SEQ ID NO: 4 - RNA, 913 nt, synthetic construct
  misc_feature 1..913: note = NOV2M-EGFP RNA
  source 1..913: mol_type = other RNA
gtaaacaaccaataacatcggcgcgccctcagcatcgattgaattggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtctagagtcgacccgggcggccgcttccacccacagaagcgttgacgacgcaaaacgtccttaaagcgttgacgacgcaaaacgtccccaagctcgtagcaccgaccctatacccatctctagggtcttcaacctcttggt

SEQ ID NO: 5 - RNA, 720 nt, synthetic construct
  misc_feature 1..720: note = EGFP
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 6 - RNA, 21 nt, synthetic construct
  misc_feature 1..21: note = NOV1 5'-UTR
  source 1..21: mol_type = other RNA
gtattgaatccaaaactcaaa

SEQ ID NO: 7 - RNA, 47 nt, synthetic construct
  misc_feature 1..47: note = NOV1 3'-UTR
  source 1..47: mol_type = other RNA
ttcatcgtcccatctgacgaaacccgaactaggcttatgccagtggt

SEQ ID NO: 8 - RNA, 43 nt, synthetic construct
  misc_feature 1..43: note = MCS
  source 1..43: mol_type = other RNA
ggcgcgccctcagcatcgattcaattgggccggccgcggccgc

SEQ ID NO: 9 - RNA, 19 nt, synthetic construct
  misc_feature 1..19: note = NOV2-5'UTR
  source 1..19: mol_type = other RNA
gtaaacaaccaataacatc

SEQ ID NO: 10 - RNA, 117 nt, synthetic construct
  misc_feature 1..117: note = NOV2-3'UTR
  source 1..117: mol_type = other RNA
ttccacccacagaagcgttgacgacgcaaaacgtccttaaagcgttgacgacgcaaaacgtccccaagctcgtagcaccgaccctatacccatctctagggtcttcaacctcttggt

SEQ ID NO: 11 - RNA, 638 nt, synthetic construct
  misc_feature 1..638: note = DENVM RNA, MULTIPLE CLONING SITE
  source 1..638: mol_type = other RNA
agttgttagtctacgtggaccgacaaagacagattctttgagggagctaagctcaacgtagttctaacagttttttaattagagagcagatctctgatcaataaccaacggaaaaaggcgaaaaacacgcctttcaatatcctgggcgcgccctcagcatcgattcaattgggccggccgcggccgcaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacaggatattgacgctgggaaacaccagagatcctgctgtctcctcagcatcattccaggcacagaacgccagaaaatggaatggtgctgttgaatcaacaggttct

SEQ ID NO: 12 - RNA, 638 nt, synthetic construct
  misc_feature 1..638: note = CAPLESS-DENVM RNA, MULTIPLE CLONING SITE
  source 1..638: mol_type = other RNA
ggttgttagtctacgtggaccgacaaagacagattctttgagggagctaagctcaacgtagttctaacagttttttaattagagagcagatctctgatcaataaccaacggaaaaaggcgaaaaacacgcctttcaatatcctgggcgcgccctcagcatcgattcaattgggccggccgcggccgcaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacaggatattgacgctgggaaacaccagagatcctgctgtctcctcagcatcattccaggcacagaacgccagaaaatggaatggtgctgttgaatcaacaggttct

SEQ ID NO: 13 - RNA, 385 nt, synthetic construct
  misc_feature 1..385: note = BYDVM RNA, MULTIPLE CLONING SITE
  source 1..385: mol_type = other RNA
ggtgaagattgaccatctcacaaaagctgttacgtgcttgtaacacactacgcgcccgttttgtattcgggaagtagttgcgaaaacggtccccttattgcctgacaagctaagggccacccttctttccccaccgccatcggcgcgccctcagcatcgattcaattgggccggccgcggccgcgtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 14 - RNA, 385 nt, synthetic construct; identical to SEQ ID NO: 13
  misc_feature 1..385: note = CAPLESS-BYDVM RNA, MULTIPLE CLONING SITE
  source 1..385: mol_type = other RNA

SEQ ID NO: 15 - RNA, 491 nt, synthetic construct
  misc_feature 1..491: note = BYDV2M RNA, MULTIPLE CLONING SITE
  source 1..491: mol_type = other RNA
ggtgaagattgaccatctcacaaaagctgttacgtgcttgtaacacactacgcgcccgttttgtattcgggaagtagttgcgaaaacggtccccttattgcctgacaagctaagggccacccttctttccccaccgccatcggcgcgccctcagcatcgattcaattgggccggccgcggccgcagacaacaccactagcacaaatcggatcctgggaaacaggcagaacttcggttcgtaagctcgggtaggccgtcaacctaccgccgtatcgtattgtgtttggccggtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 16 - RNA, 491 nt, synthetic construct; identical to SEQ ID NO: 15
  misc_feature 1..491: note = CAPLESS-BYDV2M RNA, MULTIPLE CLONING SITE
  source 1..491: mol_type = other RNA

SEQ ID NO: 17 - RNA, 388 nt, synthetic construct
  misc_feature 1..388: note = MNESVM RNA, MULTIPLE CLONING SITE
  source 1..388: mol_type = other RNA
ggagatatcgacctgcctgaccaggctgagattgcgctagccggcgtagttggtatctctcgcgcaagcgggtttgaaggtgcggcctatcttaggggggtaaattgtaacttcgcacaaaggcggcgcgccctcagcatcgattcaattgggccggccgcggccgcgacaaactctccgcctaatgtctgatgatgtgaggaacgtggactgtgatgtggtggtgcggtaccatggctggtcaccatggtaatgcgtagggcaacacagttcattaagactcactgatgatggcactaggcacggttcacccccatccttcgggagggctatagggggtgaccgggttacaccaccggaagaccggaacattgcctttgggcagccc

SEQ ID NO: 18 - RNA, 388 nt, synthetic construct; identical to SEQ ID NO: 17
  misc_feature 1..388: note = CAPLESS-MNESVM RNA, MULTIPLE CLONING SITE
  source 1..388: mol_type = other RNA

SEQ ID NO: 19 - RNA, 396 nt, synthetic construct
  misc_feature 1..396: note = PMVM RNA, MULTIPLE CLONING SITE
  source 1..396: mol_type = other RNA
ggtattggctgcaacccatacctgaagggcgcgccctcagcatcgattcaattgggccggccgcggccgcacctgcccaaggttatcacaggctaggaccggcctgataaaggtccgacttgatgtgcagcttcttaggtgcatctcgtatagaagcgctctggacatgacacctttgagacagaccgtacagcagtcacacgggacgccacaccacctttgcagaggtgcccttgggaaaccaatggtgtggggtgacactgattagtcgttaacggtgtactaatccagagtagttcagtgcaggggaaacccgggccgtaagcacgtggaactatataacttaatctcgctgagaagattgggggggattcatgaaatcccctcaccaggccc

SEQ ID NO: 20 - RNA, 396 nt, synthetic construct; identical to SEQ ID NO: 19
  misc_feature 1..396: note = CAPLESS-PMVM RNA, MULTIPLE CLONING SITE
  source 1..396: mol_type = other RNA

SEQ ID NO: 21 - RNA, 766 nt, synthetic construct
  misc_feature 1..766: note = PEMV2M RNA, MULTIPLE CLONING SITE
  source 1..766: mol_type = other RNA
ggtatttatagagatcagtggcgcgccctcagcatcgattcaattgggccggccgcggccgcggcttcgcttcccgccggaagaccgcggcggttctgttcctcccacaggagtacggcaacaacccaccttgggaaagtggggatcccagcactaactcctttaactaggcgggcgtgttggttacagtacgaggggacagtacgcattgaaactgagccccaccacaactctcatccacagggtggttgggacgcaggtgtcggagggatcgccagccctcaggatagtgagctcccgcagagggataagctgtctccctgcgacgtagtagtagaacacgtgggataggggatgaccttgtcgaccgtttgtcggtcccctgctccttagagctggcaaggcgcccattggttctacatttctaccaaagttggtggtggatgtctcgcccaaaaagatcataaacgcgcgggataaggccctctccaccttcgccgggtaaggctagagtcagcgctgcatgactataacttgcggccgatccagttgcacgactggtggtcccccccagtgtctcggtagtctgccgagtgggcggtggtcggattccaccacaccctgccacgaggtgcgtggagacttggccagtctaggctcgtcgtaattagtagcagcgacgttaatcaacccgtccgggcatacaataggaccggttgtgcttcttcctctccttcttagccaggtggttacctccctggcgccc

SEQ ID NO: 22 - RNA, 766 nt, synthetic construct; identical to SEQ ID NO: 21
  misc_feature 1..766: note = CAPLESS-PEMV2M RNA, MULTIPLE CLONING SITE
  source 1..766: mol_type = other RNA

SEQ ID NO: 23 - RNA, 433 nt, synthetic construct
  misc_feature 1..433: note = TCVM RNA, MULTIPLE CLONING SITE
  source 1..433: mol_type = other RNA
ggtaatatatgctttctacaactctctctcactggtcctcctactttgtcatctgattcctgaaatcaaaccgattcacacatcctacaacacacacgactcatcgaagcagcaacacataagcatcaacactggaaggcgcgccctcagcatcgattcaattgggccggccgcggccgctacggtaatagtgtagtcttctcatcttagtagttagctctctcttatattaagaaaagaaaacaaaacccccagtcgctttattttgacctgtgttagggaccaaaaacggtggcagcactgtctagctgcgggcattagactggaaaactagtgctctttgggtaaccactaaaatcccgaaagggtgggctgtggtgaccttccgaactaaaagatagcctccctcctcgcgcggggggggggcctgccc

SEQ ID NO: 24 - RNA, 433 nt, synthetic construct; identical to SEQ ID NO: 23
  misc_feature 1..433: note = CAPLESS-TCVM RNA, MULTIPLE CLONING SITE
  source 1..433: mol_type = other RNA

SEQ ID NO: 25 - RNA, 143 nt, synthetic construct
  misc_feature 1..143: note = DENVM 5'-UTR
  source 1..143: mol_type = other RNA
gttgttagtctacgtggaccgacaaagacagattctttgagggagctaagctcaacgtagttctaacagttttttaattagagagcagatctctgatcaataaccaacggaaaaaggcgaaaaacacgcctttcaatatcctg

SEQ ID NO: 26 - RNA, 451 nt, synthetic construct
  misc_feature 1..451: note = DENVM 3'-UTR
  source 1..451: mol_type = other RNA
aaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacaggatattgacgctgggaaacaccagagatcctgctgtctcctcagcatcattccaggcacagaacgccagaaaatggaatggtgctgttgaatcaacaggttct

SEQ ID NO: 27 - RNA, 141 nt, synthetic construct
  misc_feature 1..141: note = BYDV2M 5'-UTR
  source 1..141: mol_type = other RNA
ggtgaagattgaccatctcacaaaagctgttacgtgcttgtaacacactacgcgcccgttttgtattcgggaagtagttgcgaaaacggtccccttattgcctgacaagctaagggccacccttctttccccaccgccatc

SEQ ID NO: 28 - RNA, 307 nt, synthetic construct
  misc_feature 1..307: note = BYDV2M 3'-UTR
  source 1..307: mol_type = other RNA
agacaacaccactagcacaaatcggatcctgggaaacaggcagaacttcggttcgtaagctcgggtaggccgtcaacctaccgccgtatcgtattgtgtttggccggtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 29 - RNA, 27 nt, synthetic construct
  misc_feature 1..27: note = PMVM 5'-UTR
  source 1..27: mol_type = other RNA
ggtattggctgcaacccatacctgaag

SEQ ID NO: 30 - RNA, 326 nt, synthetic construct
  misc_feature 1..326: note = PMVM 3'-UTR
  source 1..326: mol_type = other RNA
acctgcccaaggttatcacaggctaggaccggcctgataaaggtccgacttgatgtgcagcttcttaggtgcatctcgtatagaagcgctctggacatgacacctttgagacagaccgtacagcagtcacacgggacgccacaccacctttgcagaggtgcccttgggaaaccaatggtgtggggtgacactgattagtcgttaacggtgtactaatccagagtagttcagtgcaggggaaacccgggccgtaagcacgtggaactatataacttaatctcgctgagaagattgggggggattcatgaaatcccctcaccaggccc

SEQ ID NO: 31 - RNA, 19 nt, synthetic construct
  misc_feature 1..19: note = PEMV2M 5'-UTR
  source 1..19: mol_type = other RNA
ggtatttatagagatcagt

SEQ ID NO: 32 - RNA, 704 nt, synthetic construct
  misc_feature 1..704: note = PEMV2M 3'-UTR
  source 1..704: mol_type = other RNA
ggcttcgcttcccgccggaagaccgcggcggttctgttcctcccacaggagtacggcaacaacccaccttgggaaagtggggatcccagcactaactcctttaactaggcgggcgtgttggttacagtacgaggggacagtacgcattgaaactgagccccaccacaactctcatccacagggtggttgggacgcaggtgtcggagggatcgccagccctcaggatagtgagctcccgcagagggataagctgtctccctgcgacgtagtagtagaacacgtgggataggggatgaccttgtcgaccgtttgtcggtcccctgctccttagagctggcaaggcgcccattggttctacatttctaccaaagttggtggtggatgtctcgcccaaaaagatcataaacgcgcgggataaggccctctccaccttcgccgggtaaggctagagtcagcgctgcatgactataacttgcggccgatccagttgcacgactggtggtcccccccagtgtctcggtagtctgccgagtgggcggtggtcggattccaccacaccctgccacgaggtgcgtggagacttggccagtctaggctcgtcgtaattagtagcagcgacgttaatcaacccgtccgggcatacaataggaccggttgtgcttcttcctctccttcttagccaggtggttacctccctggcgccc

SEQ ID NO: 33 - RNA, 124 nt, synthetic construct
  misc_feature 1..124: note = MNESVM 5'-UTR
  source 1..124: mol_type = other RNA
ggagatatcgacctgcctgaccaggctgagattgcgctagccggcgtagttggtatctctcgcgcaagcgggtttgaaggtgcggcctatcttaggggggtaaattgtaacttcgcacaaaggc

SEQ ID NO: 34 - RNA, 221 nt, synthetic construct
  misc_feature 1..221: note = MNESVM 3'-UTR
  source 1..221: mol_type = other RNA
gacaaactctccgcctaatgtctgatgatgtgaggaacgtggactgtgatgtggtggtgcggtaccatggctggtcaccatggtaatgcgtagggcaacacagttcattaagactcactgatgatggcactaggcacggttcacccccatccttcgggagggctatagggggtgaccgggttacaccaccggaagaccggaacattgcctttgggcagccc

SEQ ID NO: 35 - RNA, 137 nt, synthetic construct
  misc_feature 1..137: note = TCVM 5'-UTR
  source 1..137: mol_type = other RNA
ggtaatatatgctttctacaactctctctcactggtcctcctactttgtcatctgattcctgaaatcaaaccgattcacacatcctacaacacacacgactcatcgaagcagcaacacataagcatcaacactggaa

SEQ ID NO: 36 - RNA, 253 nt, synthetic construct
  misc_feature 1..253: note = TCVM 3'-UTR
  source 1..253: mol_type = other RNA
tacggtaatagtgtagtcttctcatcttagtagttagctctctcttatattaagaaaagaaaacaaaacccccagtcgctttattttgacctgtgttagggaccaaaaacggtggcagcactgtctagctgcgggcattagactggaaaactagtgctctttgggtaaccactaaaatcccgaaagggtgggctgtggtgaccttccgaactaaaagatagcctccctcctcgcgcggggggggggcctgccc

SEQ ID NO: 37 - RNA, 265 nt, synthetic construct
  misc_feature 1..265: note = SARSVM 5'-UTR
  source 1..265: mol_type = other RNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 38 - RNA, 246 nt, synthetic construct
  misc_feature 1..246: note = SARSVM 3'-UTR
  source 1..246: mol_type = other RNA
caatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaaaaaaaaaaaaaaaaaaa

SEQ ID NO: 39 - RNA, 13017 nt, synthetic construct
  misc_feature 1..13017: note = COL1A1 COL1A2 COL1A1 COLLAGEN CODING REGION RNA
  source 1..13017: mol_type = other RNA
atgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccacgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcgcatcatcgatgtggcccccttggacgttggtgccccagaccaggaattcggcttcgacgttggccatgtctgcttcctgggctccggagagggcagaggaagtctgctaacatgcggtgacgtcgaggagaatcctggcccaatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtcccgctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaagggtcgggtcaatgtactaactacgctttgttgaaactcgctggcgatgttgaaagtaaccccggtcctatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgaccggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccacgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcgcatcatcgatgtggcccccttggacgttggtgccccagaccaggaattcggcttcgacgttggccatgtctgcttcctgtaa

SEQ ID NO: 40 - RNA, 3171 nt, synthetic construct
  misc_feature 1..3171: note = EPO MULTIMERS CODING REGION RNA
  source 1..3171: mol_type = other RNA
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggttctggcgtgaaacagactttgaattttgaccttctcaagttggcgggagacgtggagtccaacccagggcccatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggctccggagagggcagaggaagtctgctaacatgcggtgacgtcgaggagaatcctggcccaatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagagggtcgggtcaatgtactaactacgctttgttgaaactcgctggcgatgttgaaagtaaccccggtcctatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagaggaagcggagctactaacttcagcctgctgaagcaggctggagacgtggaggagaaccctggacctatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagataa

SEQ ID NO: 41 - DNA, 4362 bp, synthetic construct
  misc_feature 1..4362: note = TERT/TERC RNA IN MNESVM+50A RNA
  source 1..4362: mol_type = other DNA
agatatcgacctgcctgaccaggctgagattgcgctagccggcgtagttggtatctctcgcgcaagcgggtttgaaggtgcggcctatcttaggggggtaaattgtaacttcgcacaaaggcgcgccaccatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgagcggccgcgacaaactctccgcctaatgtctgatgatgtgaggaacgtggactgtgatgtggtggtgcggtaccatggctggtcaccatggtaatgcgtagggcaacacagttcattaagactcactgatgatggcactaggcacggttcacccccatccttcgggagggctatagggggtgaccgggttacaccaccggaagaccggaacattgcctttgggcagccccaacccctgatgagtccgtgaggacgaaacggtaggaattcctaccgtcgggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgccaccggagtcgactccggtctgatgagtccgtgaggacgaagcatgtacttaagaaaaaaaaaaaaaaaaaaaaaaaaaaaaaaaaaaaaaaaaaaaaaaaaaa

SEQ ID NO: 42 - RNA, 11231 nt, synthetic construct
  source 1..11231: mol_type = other RNA
  misc_feature 1..11231: note = NOV2M-DMD+28A mRNA
gtaaacaaccaataacatcgcgatcgccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaattccacccacagaagcgttgacgacgcaaaacgtccttaaagcgttgacgacgcaaaacgtccccaagctcgtagcaccgaccctatacccatctctagggtcttcaacctcttggtaaaaaaaaaaaaaaaaaaaaaaaaaaaa